(19) United States
(12) Patent Application Publication
(10) Pub. No.: US 2022/0192741 A1
(43) Pub. Date: Jun. 23, 2022

(54) SYSTEMS AND METHODS FOR TREATING TISSUE WITH PULSED FIELD ABLATION

(71) Applicant: KARDIUM INC., Burnaby (CA)

(72) Inventors: Daniel Martin Reinders, Richmond (CA); Shane Fredrick Miller-Tait, North Vancouver (CA)

(21) Appl. No.: 17/521,107

(22) Filed: Nov. 8, 2021

Related U.S. Application Data
(60) Provisional application No. 63/129,806, filed on Dec. 23, 2020.

(51) Int. Cl. A61B 18/14

(52) U.S. Cl. CPC A61B 18/1492; A61B 2018/00351

(57) ABSTRACT

A pulsed field ablation system may be configured to control characteristics of high voltage pulses delivered during one or more cardiac cycles based on a characteristic of one or more cardiac cycles. For example, if a particular cardiac cycle has a first characteristic, a first high voltage pulse train may be delivered, but if the particular cardiac cycle has a second characteristic different than the first characteristic, a second high voltage pulse train having at least a different characteristic than the first high voltage pulse may instead be delivered.

### CROSS-REFERENCE TO RELATED APPLICATION

This application claims the benefit of U.S. Provisional Application No. 63/129,806, filed Dec. 23, 2020, the entire disclosure of which is hereby incorporated herein by reference.

### TECHNICAL FIELD

Aspects of this disclosure generally are related to systems and methods for treating tissue using pulsed field ablation.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with open heart methods using a technique known as the “Cox-Maze procedure”. During this procedure, physicians create specific patterns of lesions in the left or right atria to block various paths taken by the spurious electrical signals. Such lesions were originally created using incisions, but are now typically created by ablating the tissue with various techniques including radio-frequency (RF) energy, microwave energy, laser energy, and cryogenic techniques. Although RF ablation techniques are commonly employed in cardiac applications, possible complications may arise from the thermal energy that is delivered. For example, this thermal energy may cause direct damage to the target cardiac tissue including tissue charring and steam pops, thermal coagulation of blood which may lead to strokes, and damage to various anatomical structures proximate the heart such as the phrenic nerve or esophagus.

Recently, pulsed field ablation (“PFA”) techniques have been investigated in various tissue ablation procedures. In PFA, high voltage pulses with sub-second pulse durations are applied to target tissue. In some cases, the high voltage pulses form pores in cell membranes in a procedure sometimes referred to as electroporation. When the electroporation process is such that the formed pores are permanent in nature and result in cell death, the process is referred to as irreversible electroporation by some. When the electroporation process is such that the formed pores are temporary in nature, and the cell survives the electroporation process, the process is referred to as reversible electroporation by some. Pulsed field ablation, because it refers to ablation of tissue, typically involves irreversible electroporation of target tissue. In some cases, PFA shows a specificity for certain tissues. For example, it has been shown that tissue such as myocardium tissue is highly susceptible to necrosis under the effects of PFA, while collateral structures such as the esophagus and phrenic nerve seem to be relatively resistant to injury.

Although PFA is considered by some to be a generally non-thermal method for causing cell death, the present inventors recognized that the use of various PFA protocols can cause some degree of potential thermal damage to tissue of the desired ablation region. The present inventors recognized that this potential thermal damage may become a particularly important consideration when relatively high PFA voltages and/or relatively large numbers of pulses are employed to ablate tissue.

The present inventors recognized that Joule heating of a conductive medium when exposed to electrical energy causes a rise in temperature, thereby making temperature increases an inevitable outcome during PFA. Accordingly, the present inventors recognized that a desired objective for PFA may be to avoid additional potential thermal effects due to Joule heating. In other words, there may be a desire to avoid causing thermally induced tissue damage beyond the non-thermal PFA lesions in typical PFA procedures. However, the present inventors recognized that maximizing the efficacy of the PFA procedures typically entails delivering a maximum number of pulses in a period of time, and clinical demands indicate that these pulses should be delivered as quickly as is safely possible to reduce procedure times. The present inventors recognized that these circumstances provide an incentive to provide a high density of pulse energy per unit time up to a safe limit. However, the present inventors recognized that this push for high density of pulse energy per unit time during PFA can increase the risk for undesirable thermal effects.

It is noted that what constitutes undesirable thermal effects during PFA can vary depending on the context. In some cases, high voltage pulses applied within the vascular system may increase the risk of thermally coagulating blood and causing downstream embolism. In some cases, the present inventors recognized that thermal damage to tissue adjacent target tissue may negate the benefit of PFA for the target tissue. For example, it has been observed that the tolerance of the esophagus to PFA is much higher than that of myocardial tissue. However, if the PFA application were to cause thermal damage to both the esophagus and the myocardium, then the safety benefit of PFA within this context may be negated. In some cases, the present inventors recognized that thermal effects may also lead to excess microbubble production due to electrode edge heating causing focal steam bubbles (or explosive steam pop even) or nitrogen or electrolytic gas byproduct bubbles due to gas supersaturation caused by decreased solubility at higher temperatures. Such bubble formation may cause an undesirable increase in the risk of stroke or other adverse consequences. The present inventors recognized that undesirable thermal effects may also include effects on pulse parameters employed in PFA. For example, the electrical conductivity of blood and tissue typically increases approximately two percent per degree Celsius. Accordingly, the present inventors recognized that elevated tissue or blood temperatures can therefore lead to an increase in the delivered current when a constant voltage is maintained. This increase in current in turn may promote electrolysis microbubble formation (primarily oxygen, hydrogen, and chlorine gas when pulsing within blood) due to the higher current density over the electrode surface required in order to achieve the target voltage gradients within tissue, as recognized by the present inventors.

The present inventors have recognized that these circumstances are obstacles to maintaining efficacious PFA treatments in which the predominant cell death mechanism is caused by electroporation while also maintaining sufficiently low temperatures to prevent damage to vital structures. In this regard, the present inventors recognized that there is a need in the art for PFA systems with improved safety, efficiency, and/or effectiveness that reduce undesired potential thermal effects or have other benefits.

### SUMMARY

At least the above-discussed need is addressed and technical solutions are achieved in the art by various embodiments of the present invention. In some embodiments, a pulsed field ablation system may be summarized as including, according to various embodiments, an input-output device system, a memory device system storing a program, and a data processing device system communicatively connected to the input-output device system and the memory device system. According to various embodiments, the data processing device system may be configured at least by the program at least to cause delivery, via the input-output device system and via a first pulsed field ablation transducer located on a catheter device, of a respective high voltage pulse train during each respective cardiac cycle of a plurality of cardiac cycles including at least a first cardiac cycle and a second cardiac cycle. According to various embodiments, each respective high voltage pulse train defines a plurality of high voltage pulses. According to various embodiments, each respective high voltage pulse train is configured to cause pulsed field ablation of tissue. According to various embodiments, the data processing device system may be configured at least by the program at least to cause delivery of each respective high voltage pulse train only during a particular time interval in the respective cardiac cycle. According to various embodiments, the particular time intervals in the first cardiac cycle and the second cardiac cycle may be configured such that a first ratio of the duration of the particular time interval in the first cardiac cycle to the duration of the first cardiac cycle is different than a second ratio of the duration of the particular time interval in the second cardiac cycle to the duration of the second cardiac cycle. In some embodiments, the high voltage pulses of the respective high voltage pulse train which the data processing device system is configured to cause delivery of during the first cardiac cycle may be configured to cumulatively deliver first energy during the particular time interval in the first cardiac cycle, and the high voltage pulses of the respective high voltage pulse train which the data processing device system is configured to cause delivery of during the second cardiac cycle are configured to cumulatively deliver second energy during the particular time interval in the second cardiac cycle. In various embodiments, the second energy is different than the first energy.

In some embodiments, the first ratio is less than the second ratio, and the first energy is greater than the second energy. In some embodiments, the duration of the first cardiac cycle is longer than the duration of the second cardiac cycle, and the first energy is greater than the second energy.

In some embodiments, the duration of the particular time interval in the first cardiac cycle is the same as the duration of the particular time interval in the second cardiac cycle. In some embodiments, the duration of the first cardiac cycle is different than the duration of the second cardiac cycle. In some embodiments, the first ratio is less than the second ratio, and the first energy is greater than the second energy. In some embodiments, the duration of the first cardiac cycle is longer than the duration of the second cardiac cycle, and the first energy is greater than the second energy. In some embodiments, a duration of at least one of the particular time intervals may shorter than a duration of the respective cardiac cycle. In some embodiments, a duration of each particular time interval of the particular time intervals in the first cardiac cycle and the second cardiac cycle may be shorter than a duration of the respective cardiac cycle.

According to various embodiments, the particular time interval in the first cardiac cycle has a first determined temporal relationship with a particular cardiac event in the first cardiac cycle, and the particular time interval in the second cardiac cycle has a second determined temporal relationship with a particular cardiac event in the second cardiac cycle. In some embodiments, the first determined temporal relationship may be the same as the second determined temporal relationship. In some embodiments, the particular time interval in the first cardiac cycle may occur during a refractory period in the first cardiac cycle, and the particular time interval in the second cardiac cycle may occur during a refractory period in the second cardiac cycle.

According to some embodiments, the respective high voltage pulse train which the data processing device system is configured to cause delivery of during the first cardiac cycle may be configured to have a first particular number of high voltage pulses, and the respective high voltage pulse train which the data processing device system is configured to cause delivery of during the second cardiac cycle may be configured to have a second particular number of high voltage pulses, the second particular number of high voltage pulses different than the first particular number of high voltage pulses. In some embodiments, the first ratio is less than the second ratio, and the first particular number of high voltage pulses is greater than the second particular number of high voltage pulses. In some embodiments, the duration of the first cardiac cycle is longer than the duration of the second cardiac cycle, and the first particular number of high voltage pulses is greater than the second particular number of high voltage pulses.

According to various embodiments, the respective high voltage pulse train which the data processing device system is configured to cause delivery of during the first cardiac cycle is configured to have a first inter-pulse spacing between adjacent high voltage pulses in the respective high voltage pulse train, and the respective high voltage pulse train which the data processing device system is configured to cause delivery of during the second cardiac cycle is configured to have a second inter-pulse spacing between adjacent high voltage pulses in the respective high voltage pulse train. According to various embodiments, the second inter-pulse spacing may be different than the first inter-pulse spacing.

According to various embodiments, each of at least one high voltage pulse in the respective high voltage pulse train which the data processing device system is configured to cause delivery of during the first cardiac cycle is configured to deliver a respective first amount of pulse energy, and each of at least one high voltage pulse in the respective high voltage pulse train which the data processing device system is configured to cause delivery of during the second cardiac cycle is configured to deliver a respective second amount of pulse energy. According to various embodiments, each respective second amount of pulse energy may be different than each respective first amount of pulse energy.

According to various embodiments, each of at least one high voltage pulse in the respective high voltage pulse train which the data processing device system is configured to cause delivery of during the first cardiac cycle is configured to have a respective first pulse shape, and each of at least one high voltage pulse in the respective high voltage pulse train which the data processing device system is configured to cause delivery of during the second cardiac cycle is configured to have a respective second pulse shape. According to various embodiments, each respective second pulse shape may be different than each respective first pulse shape.

In some embodiments, one of (a) a ratio of the first energy to the duration of the first cardiac cycle and (b) a ratio of the second energy to the duration of the second cardiac cycle may be within 10% of the other of (a) and (b). In some embodiments, the respective high voltage pulse train which the data processing device system is configured to cause delivery of during the first cardiac cycle may be configured to cause delivery of a first average power during the first cardiac cycle, and the respective high voltage pulse train which the data processing device system is configured to cause delivery of during the second cardiac cycle may be configured to cause delivery of a second average power that maintains the first average power. In some embodiments, the second average power may maintain the first average power by being within 10% of the first average power. In some embodiments, the respective high voltage pulse trains delivered during the respective cardiac cycles of the plurality of cardiac cycles are a plurality of high voltage pulse trains, and each high voltage pulse in each high voltage pulse train of the plurality of high voltage pulse trains may be a high voltage pulse of at least 150 volts.

Various systems may include combinations and subsets of all the systems summarized above or otherwise described herein.

A pulsed field ablation system may be summarized as including, according to various embodiments, an input-output device system, a memory device system storing a program, and a data processing device system communicatively connected to the input-output device system and the memory device system. According to various embodiments, the data processing device system may be configured at least by the program at least to cause delivery, via the input-output device system and via a first pulsed field ablation transducer located on a catheter device, of a respective high voltage pulse train during each respective cardiac cycle of a plurality of cardiac cycles. According to various embodiments, each respective high voltage pulse train defines a plurality of high voltage pulses. According to various embodiments, each respective high voltage pulse train is configured to cause pulsed field ablation of tissue. In some embodiments, the data processing device system may be configured at least by the program at least to cause each respective high voltage pulse train to be deliverable only during a particular time interval in the respective cardiac cycle. According to various embodiments, the data processing device system may be configured at least by the program at least to cause, in response to reception of information indicative of a particular cardiac event occurring in each of at least some of the plurality of cardiac cycles, a change in at least one high voltage pulse train parameter to cause at least two of the respective high voltage pulse trains to be different from each other.

In some embodiments, each high voltage pulse in the each respective high voltage pulse train is configured to deliver a respective amount of pulse energy, and the changing in at least one high voltage pulse train parameter may be configured to cause a change in the respective amount of pulse energy that is delivered by each of at least one high voltage pulse in at least one high voltage pulse train of the at least two of the respective high voltage pulse trains. In some embodiments, the changing in at least one high voltage pulse train parameter may include a change in the number of high voltage pulses in at least one high voltage pulse train of the at least two of the respective high voltage pulse trains. In some embodiments, the changing in at least one high voltage pulse train parameter may include a change in an inter-pulse spacing between the high voltage pulses in at least one high voltage pulse train of the at least two of the respective high voltage pulse trains. In some embodiments, the changing in at least one high voltage pulse train parameter may include a change in a pulse shape in each of one or more high voltage pulses in at least one high voltage pulse train of the at least two of the respective high voltage pulse trains.

According to some embodiments, a duration of each particular time interval may be shorter than a duration of the respective cardiac cycle. In some embodiments, the particular time intervals in the respective cardiac cycles associated with the at least two of the respective high voltage pulse trains may have a same duration. In some embodiments, the respective cardiac cycles associated with the at least two of the respective high voltage pulse trains may have different durations. In some embodiments, the particular time interval in the respective cardiac cycle associated with each of the at least two of the respective high voltage pulse trains may occur during a refractory period in each of the respective cardiac cycles associated with each of the at least two of the respective high voltage pulse trains.

According to some embodiments, the at least two of the respective high voltage pulse trains may include a first high voltage pulse train and a second high voltage pulse train. In some embodiments, a first ratio of a duration of the particular time interval in the respective cardiac cycle associated with the first high voltage pulse train to a duration of the respective cardiac cycle associated with the first high voltage pulse train may be different than a second ratio of a duration of the particular time interval in the respective cardiac cycle associated with the second high voltage pulse train to a duration of the respective cardiac cycle associated with the second high voltage pulse train.

In some embodiments, the at least two of the respective high voltage pulse trains may include a first high voltage pulse train and a second high voltage pulse train, and the high voltage pulses of the first high voltage pulse train are configured to cumulatively deliver a first energy during the particular time interval of the respective cardiac cycle, and the high voltage pulses of the second high voltage pulse train are configured to cumulatively deliver a second energy during the particular time interval of the respective cardiac cycle. In some embodiments, the second energy may be different than the first energy. In some embodiments, the particular time interval in the respective cardiac cycle associated with the first high voltage pulse train may have a first temporal relationship with an occurrence of the particular cardiac event in the respective cardiac cycle associated with the first high voltage pulse train, and the particular time interval in the respective cardiac cycle associated with the second high voltage pulse train may have a second temporal relationship with an occurrence of the particular cardiac event in the respective cardiac cycle associated with the second high voltage pulse train. In some embodiments, the first temporal relationship and the second temporal relationship may be a same temporal relationship. In some embodiments, a first ratio of a duration of the particular time interval in the respective cardiac cycle associated with the first high voltage pulse train to a duration of the respective cardiac cycle associated with the first high voltage pulse train may be different than a second ratio of a duration of the particular time interval in the respective cardiac cycle associated with the second high voltage pulse train to a duration of the respective cardiac cycle associated with the second high voltage pulse train. According to some embodiments, the first energy may be greater than the second energy, and the first ratio may be less than the second ratio. In some embodiments, the duration of the respective cardiac cycle associated with the first high voltage pulse train may be different than the duration of the respective cardiac cycle associated with the second high voltage pulse train. In some embodiments, the duration of the particular time interval in the respective cardiac cycle associated with the first high voltage pulse train and the duration of the particular time interval in the respective cardiac cycle associated with the second high voltage pulse train may be the same. In some embodiments, the first energy may be greater than the second energy, and the duration of the respective cardiac cycle associated with the first high voltage pulse train may be greater than the duration of the respective cardiac cycle associated with the second high voltage pulse train. In some embodiments, one of a first ratio of the first energy to the duration of the respective cardiac cycle associated with the first high voltage pulse train and a second ratio of the second energy to the duration of the respective cardiac cycle associated with the second high voltage pulse train may be within 10% of the other of the first ratio and the second ratio. In some embodiments, the data processing device system may be configured at least by the program at least to cause the first high voltage pulse train to deliver a first average power during the respective cardiac cycle, and cause the second high voltage pulse train to deliver a second average power during the respective cardiac cycle, the second average power configured to maintain the first average power. In some embodiments, the second average power may maintain the first average power by being within 10% of the first average power.

According to some embodiments, a duration of the particular time interval in the respective cardiac cycle associated with the first high voltage pulse train may be shorter than a duration of the respective cardiac cycle associated with the first high voltage pulse train, and a duration of the particular time interval in the respective cardiac cycle associated with the second high voltage pulse train may be shorter than a duration of the respective cardiac cycle associated with the second high voltage pulse train.

In some embodiments, the information indicative of a particular cardiac event occurring in each of at least some of the plurality of cardiac cycles may indicate at least an occurrence of the particular cardiac event occurring in the respective cardiac cycle associated with one of the at least two respective high voltage pulse trains. In some embodiments, each of the at least some of the plurality of cardiac cycles may occur prior to at least one of the respective cardiac cycles associated with the at least two respective high voltage pulse trains.

In some embodiments, the information indicative of the particular cardiac event occurring in each of at least some of the plurality of cardiac cycles may indicate an occurrence of the particular cardiac event in each cardiac cycle of a group of consecutively occurring cardiac cycles of the plurality of cardiac cycles. In some embodiments, the data processing device system may be configured at least by the program at least to determine one or more cardiac cycle durations based at least on the indicated occurrence of the particular cardiac event in each cardiac cycle of the group of consecutively occurring cardiac cycles of the plurality of cardiac cycles. In some embodiments, the data processing device system may be configured at least by the program to cause the change in the at least one high voltage pulse train parameter based at least on the determined one or more cardiac cycle durations.

In some embodiments, the particular cardiac event may be at least part of a QRS complex. In some embodiments, the particular cardiac event may be a cardiac pulse caused by a pacing signal deliverable to a patient by a pacing device system. In some embodiments, each high voltage pulse in each respective high voltage pulse train may be a high voltage pulse of at least 150 volts.

Various systems may include combinations and subsets of all the systems summarized above or otherwise described herein.

According to some embodiments, a pulsed field ablation system may be summarized as including, according to various embodiments, an input-output device system, a memory device system storing a program, and a data processing device system communicatively connected to the input-output device system and the memory device system. According to various embodiments, the data processing device system may be configured at least by the program at least to cause, in association with a first state in which at least a particular cardiac cycle of a patient is determined to have a first duration, delivery, via the input-output device system and via a first pulsed field ablation transducer located on a catheter device, of a first high voltage pulse train during a first particular time interval. According to various embodiments, a duration of the first particular time interval may be less than the first duration. According to various embodiments, the first high voltage pulse train may define a first plurality of high voltage pulses, and the first high voltage pulse train may be configured to cause pulsed field ablation of tissue. In some embodiments, the first plurality of high voltage pulses may be configured to cumulatively deliver first energy during the first particular time interval. According to various embodiments, the data processing device system may be configured at least by the program at least to cause, in association with a second state in which at least the particular cardiac cycle of the patient is determined to have a second duration different than the first duration, delivery, via the input-output device system and via the first pulsed field ablation transducer, of a second high voltage pulse train during a second particular time interval. In some embodiments, a duration of the second particular time interval may be less than the second duration. In some embodiments, the second high voltage pulse train may define a second plurality of high voltage pulses, and the second high voltage pulse train may be configured to cause pulsed field ablation of tissue. In some embodiments, the second plurality of high voltage pulses may be configured to cumulatively deliver second energy during the second particular time interval. In some embodiments, the second energy may be different than the first energy.

In some embodiments, the second duration may be shorter than the first duration, and the second energy may be less than the first energy. In some embodiments, the data processing device system may be configured at least by the program at least to cause the delivery, in association with the first state, of the first high voltage pulse train during the particular cardiac cycle. In some embodiments, the data processing device system may be configured at least by the program at least to cause the delivery, in association with the second state, of the second high voltage pulse train during the particular cardiac cycle. In some embodiments, each of the first particular time interval and the second particular time interval may have a determined temporal relationship with a particular cardiac event in the particular cardiac cycle. In some embodiments, the first particular time interval and the second particular time interval may have a same temporal relationship with a particular cardiac event in the particular cardiac cycle. In some embodiments, each of the first particular time interval and the second particular time interval may occur during a refractory period in the particular cardiac cycle. In some embodiments, the data processing device system may be configured at least by the program at least to cause, in association with the first state, the first high voltage pulse train to deliver a first average power during the particular cardiac cycle, and cause, in association with the second state, the second high voltage pulse train to deliver a second average power during the particular cardiac cycle. In some embodiments, the second average power may be within 10% of the first average power.

In some embodiments, the data processing device system may be configured at least by the program at least to cause the delivery, in association with the first state, of the first high voltage pulse train during a second particular cardiac cycle subsequent to the particular cardiac cycle. In some embodiments, the data processing device system may be configured at least by the program at least to cause the delivery, in association with the second state, of the second high voltage pulse train during the second particular cardiac cycle subsequent to the particular cardiac cycle. In some embodiments, (a) a duration of the first high voltage pulse train, (b) a duration of the second high voltage pulse train, or each of (a) and (b) may be less than a duration of the second particular cardiac cycle. In some embodiments, each of the first particular time interval and the second particular time interval may have a determined temporal relationship with a particular cardiac event in the second particular cardiac cycle. In some embodiments, the first particular time interval and the second particular time interval may have a same temporal relationship with a particular cardiac event in the second particular cardiac cycle. In some embodiments, each of the first particular time interval and the second particular time interval may occur during a refractory period in the second particular cardiac cycle. In some embodiments, a ratio of the first energy to a duration of the second particular cycle is a first ratio, and a ratio of the second energy to the duration of the second particular cardiac cycle is a second ratio. In some embodiments, one of the first ratio and the second ratio may be within 10% of the other of the first ratio and the second ratio. In some embodiments, the data processing device system may be configured at least by the program at least to cause, in association with the first state, the first high voltage pulse train to deliver a first average power during the second particular cardiac cycle, and cause, in association with the second state, the second high voltage pulse train to deliver, during the second particular cardiac cycle a second average power. In some embodiments, the second average power maintains the first average power. In some embodiments, the second average power may maintain the first average power by being within 10% of the first average power.

In some embodiments, the first particular time interval may be the second particular time interval. In some embodiments, the duration of the first particular time interval may be the same as the duration of the second particular time interval. In some embodiments, each of the first particular time interval and the second particular time interval may be an uninterrupted time interval.

According to some embodiments, the data processing device system may be configured at least by the program at least to cause, in association with the first state, the first high voltage pulse train to have a first particular number of high voltage pulses. In some embodiments the data processing device system may be configured at least by the program at least to cause, in association with the second state, the second high voltage pulse train to have a second particular number of high voltage pulses. In some embodiments, the second particular number of high voltage pulses may be different than the first particular number of high voltage pulses.

In some embodiments, the data processing device system is configured at least by the program at least to cause, in association with the first state, the first high voltage pulse train to have a first particular number of high voltage pulses during delivery of the first high voltage pulse train during the first particular time interval. In some embodiments, the data processing device system may be configured at least by the program at least to cause, in association with the second state, the second high voltage pulse train to have a second particular number of high voltage pulses during delivery of the second high voltage pulse train during the second particular time interval. In some embodiments, the second particular number of high voltage pulses may be fewer than the first particular number of high voltage pulses.

In some embodiments, an inter-pulse spacing between adjacent high voltage pulses in the first high voltage pulse train may be different than an inter-pulse spacing between adjacent high voltage pulses in the second high voltage pulse train. In some embodiments, the data processing device system may be configured at least by the program at least to cause, in association with the first state, each of at least one high voltage pulse in the first high voltage pulse train to deliver a respective first amount of pulse energy. In some embodiments, the data processing device system may be configured at least by the program at least to cause, in association with the second state, each of at least one high voltage pulse in the second high voltage pulse train to deliver a respective second amount of pulse energy. In some embodiments, each respective second amount of pulse energy may be different than each respective first amount of pulse energy.

In some embodiments, the data processing device system may be configured at least by the program at least to cause, in association with the first state, each high voltage pulse in the first high voltage pulse train to deliver a respective first amount of pulse energy. In some embodiments, the data processing device system may be configured at least by the program at least to cause, in association with the second state, each high voltage pulse in the second high voltage pulse train to deliver a respective second amount of pulse energy. In some embodiments, each respective second amount of pulse energy may be different than each respective first amount of pulse energy.

In some embodiments, the data processing device system may be configured at least by the program at least to cause, in association with the first state, each of at least one high voltage pulse in the first high voltage pulse train to have a respective first pulse shape. In some embodiments, the data processing device system may be configured at least by the program at least to cause, in association with the second state, each of at least one high voltage pulse in the second high voltage pulse train to have a respective second pulse shape. In some embodiments, each respective second pulse shape may be different than each respective first pulse shape.

In some embodiments, the data processing device system may be configured at least by the program at least to cause, in association with the first state, each high voltage pulse in the first high voltage pulse train to have a respective first pulse shape. In some embodiments, the data processing device system may be configured at least by the program at least to cause, in association with the second state, each high voltage pulse in the second high voltage pulse train to have a respective second pulse shape. In some embodiments, each respective second pulse shape may be different than each respective first pulse shape.

In some embodiments, one of a first ratio of the first energy to the first duration of the particular cardiac cycle and a second ratio of the second energy to the second duration of the particular cardiac cycle may be within 10% of the other of the first ratio of the first energy to the first duration of the particular cardiac cycle and the second ratio of the second energy to the second duration of the particular cardiac cycle. In some embodiments, one of a first ratio of the first energy to an actual duration of the particular cardiac cycle and a second ratio of the second energy to the actual duration of the particular cardiac cycle may be within 10% of the other of the first ratio of the first energy to the actual duration of the particular cardiac cycle and the second ratio of the second energy to the actual duration of the particular cardiac cycle.

In some embodiments, each high voltage pulse in the first high voltage pulse train and each high voltage pulse in the second high voltage pulse train may be a high voltage pulse of at least 150 volts.

Various systems may include combinations and subsets of all the systems summarized above or otherwise described herein.

A pulsed field ablation system may be summarized as including, according to various embodiments, as including an input-output device system, a memory device system storing a program, and a data processing device system communicatively connected to the input-output device system and the memory device system. In some embodiments, the data processing device system may be configured at least by the program at least to cause, in association with a first state in which at least a particular cardiac cycle of a patient has a first characteristic, a first pulsed field ablation transducer located on a catheter device to deliver a plurality of first high voltage pulses during a first sequence of consecutive cardiac cycles. In some embodiments, the plurality of first high voltage pulses may be configured to deliver a particular average power throughout a duration of the first sequence of consecutive cardiac cycles. In some embodiments, the data processing device system may be configured at least by the program at least to cause, in association with a second state in which at least the particular cardiac cycle of a patient has a second characteristic different than the first characteristic, the first pulsed field ablation transducer to deliver a plurality of second high voltage pulses during a second sequence of consecutive cardiac cycles. In some embodiments, the delivery of the plurality of second high voltage pulses during the second sequence of consecutive cardiac cycles in association with the second state may be configured to maintain the particular average power delivered by the first pulsed field ablation transducer throughout the duration of the first sequence of consecutive cardiac cycles in association with the first state. According to various embodiments, each of the plurality of first high voltage pulses and the plurality of second high voltage pulses may be configured to cause pulsed field ablation of tissue. In some embodiments, a first particular ratio of a total number of the first high voltage pulses to a total number of cardiac cycles in the first sequence of consecutive cardiac cycles may be different than a second particular ratio of a total number of the second high voltage pulses to a total number of cardiac cycles in the second sequence of consecutive cardiac cycles.

In some embodiments, the data processing device system may be configured at least by the program at least to cause (a) in association with the first state, the first pulsed field ablation transducer to deliver a respective subset of the plurality of first high voltage pulses during each cardiac cycle of the first sequence of consecutive cardiac cycles, and (b) in association with the second state, the first pulsed field ablation transducer to deliver a respective subset of the plurality of second high voltage pulses during each cardiac cycle of the second sequence of consecutive cardiac cycles. In some embodiments, the number of first high voltage pulses in each of at least one of the respective subsets of the plurality of first high voltage pulses may be different than the number of second high voltage pulses in each of at least one of the respective subsets of the plurality of second high voltage pulses. In some embodiments, each of at least one of the respective subsets of the plurality of first high voltage pulses has a first number of the plurality of first high voltage pulses, and each of at least one of the respective subsets of the plurality of second high voltage pulses has a second number of the plurality of second high voltage pulses. In some embodiments, the second number may be different than the first number. In some embodiments, the number of first high voltage pulses in each of the respective subsets of the plurality of first high voltage pulses may be different than the number of second high voltage pulses in each of the respective subsets of the plurality of second high voltage pulses. In some embodiments, the first high voltage pulses in each of at least one of the respective subsets of the plurality of first high voltage pulses are configured to cumulatively deliver first energy during the respective cardiac cycle of the first sequence of consecutive cardiac cycles, and the second high voltage pulses in each of at least one of the respective subsets of the plurality of second high voltage pulses are configured to cumulatively deliver second energy during the respective cardiac cycle of the second sequence of consecutive cardiac cycles. In some embodiments, the second energy may be different than the first energy.

In some embodiments, the data processing device system may be configured at least by the program at least to cause (a) in association with the first state, the first pulsed field ablation transducer to deliver a respective subset of the plurality of first high voltage pulses during each cardiac cycle of some, but not all, of the cardiac cycles of the first sequence of consecutive cardiac cycles, the some, but not all, of the first sequence of consecutive cardiac cycles excluding at least one cardiac cycle of the first sequence of consecutive cardiac cycles during which no pulsed field ablation energy is delivered by the first pulsed field ablation transducer, and the excluded at least one cardiac cycle of the first sequence of consecutive cardiac cycles occurring between at least two cardiac cycles of the some, but not all, of the first sequence of consecutive cardiac cycles, or (b) in association with the second state, the first pulsed field ablation transducer to deliver a respective subset of the plurality of second high voltage pulses during each cardiac cycle of some, but not all, of the cardiac cycles of the second sequence of consecutive cardiac cycles, the some, but not all, of the second sequence of consecutive cardiac cycles excluding at least one cardiac cycle of the second sequence of consecutive cardiac cycles during which no pulsed field ablation energy is delivered by the first pulsed field ablation transducer, the excluded at least one cardiac cycle of the second sequence of consecutive cardiac cycles occurring between at least two cardiac cycles of the some, but not all, of the second sequence of consecutive cardiac cycles, or (c) both of (a) and (b).

In some embodiments, the data processing device system may be configured at least by the program at least to cause (a) in association with the first state, the first pulsed field ablation transducer to deliver a respective subset of the plurality of first high voltage pulses during each cardiac cycle of the first sequence of consecutive cardiac cycles, and (b) in association with the second state, the first pulsed field ablation transducer to deliver a respective subset of the plurality of second high voltage pulses during each cardiac cycle of some, but not all, of the cardiac cycles of the second sequence of consecutive cardiac cycles, the some, but not all, of the second sequence of consecutive cardiac cycles excluding at least one cardiac cycle of the second sequence of consecutive cardiac cycles during which no pulsed field ablation energy is delivered by the first pulsed field ablation transducer. In some embodiments, the excluded at least one cardiac cycle of the second sequence of consecutive cardiac cycles may occur between at least two cardiac cycles of the some, but not all, of the second sequence of consecutive cardiac cycles. In some embodiments, the number of first high voltage pulses in each of at least one of the respective subsets of the plurality of first high voltage pulses may be the same as the number of second high voltage pulses in each of at least one of the respective subsets of the plurality of second high voltage pulses. In some embodiments, the number of first high voltage pulses in each of at least one of the respective subsets of the plurality of first high voltage pulses may be different than the number of second high voltage pulses in each of at least one of the respective subsets of the plurality of second high voltage pulses. In some embodiments, the number of first high voltage pulses in each of the respective subsets of the plurality of first high voltage pulses may be the same as the number of second high voltage pulses in each of the respective subsets of the plurality of second high voltage pulses. In some embodiments, the number of first high voltage pulses in each of the respective subsets of the plurality of first high voltage pulses may be different than the number of second high voltage pulses in each of the respective subsets of the plurality of second high voltage pulses. In some embodiments, the first high voltage pulses in each of at least one of the respective subsets of the plurality of first high voltage pulses are configured to cumulatively deliver first energy during the respective cardiac cycle of the first sequence of consecutive cardiac cycles, and the second high voltage pulses in each of at least one of the respective subsets of the plurality of second high voltage pulses are configured to cumulatively deliver second energy during the respective cardiac cycle of the second sequence of consecutive cardiac cycles. In some embodiments, the second energy may be the same as the first energy. In some embodiments, the first high voltage pulses in each of at least one of the respective subsets of the plurality of first high voltage pulses are configured to cumulatively deliver first energy during the respective cardiac cycle of the first sequence of consecutive cardiac cycles, and the second high voltage pulses in each of at least one of the respective subsets of the plurality of second high voltage pulses are configured to cumulatively deliver second energy during the respective cardiac cycle of the second sequence of consecutive cardiac cycles. In some embodiments, the second energy may be different than the first energy.

In some embodiments, the first high voltage pulses of the plurality of first high voltage pulses are configured to cumulatively deliver first energy throughout the first sequence of consecutive cardiac cycles, and the second high voltage pulses of the plurality of second high voltage pulses are configured to cumulatively deliver second energy during the second sequence of consecutive cardiac cycles. In some embodiment, a third particular ratio of the first energy to the total number of cardiac cycles in the first sequence of consecutive cardiac cycles may be different than a fourth particular ratio of the second energy to the total number of cardiac cycles in the second sequence of consecutive cardiac cycles.

In some embodiments, the plurality of first high voltage pulses includes a plurality of subsets of the first high voltage pulses, each subset of the first high voltage pulses deliverable during a respective cardiac cycle of at least some of the cardiac cycles in the first sequence of consecutive cardiac cycles, and the plurality of second high voltage pulses includes a plurality of subsets of the second high voltage pulses, each subset of the second high voltage pulses deliverable during a respective cardiac cycle of at least some of the cardiac cycles in the second sequence of consecutive cardiac cycles. In some embodiments, in association with the first state, the data processing device system may be configured at least by the program at least to cause each respective subset of the plurality of first high voltage pulse trains to be deliverable only during a first particular time interval in the respective cardiac cycle of the at least some of the cardiac cycles in the first sequence of consecutive cardiac cycles, a duration of each first particular time interval shorter than a duration of the respective cardiac cycle of the at least some of the cardiac cycles in the first sequence of consecutive cardiac cycles. In some embodiments, in association with the second state, the data processing device system may be configured at least by the program at least to cause each respective subset of the plurality of second high voltage pulse trains to be deliverable only during a second particular time interval in the respective cardiac cycle of the at least some of the cardiac cycles in the second sequence of consecutive cardiac cycles, a duration of each second particular time interval shorter than a duration of the respective cardiac cycle of the at least some of the cardiac cycles in the second sequence of consecutive cardiac cycles. In some embodiments, the duration of each first particular time interval may be configured to be the same or substantially the same as the duration of each second particular time interval. In some embodiments, for each respective cardiac cycle of the at least some of the cardiac cycles in the first sequence of consecutive cardiac cycles, the first particular time interval has a first temporal relationship with a particular cardiac event in the respective cardiac cycle of the at least some of the cardiac cycles in the first sequence of consecutive cardiac cycles, and for each respective cardiac cycle of the at least some of the cardiac cycles in the second sequence of consecutive cardiac cycles, the second particular time interval has a second temporal relationship with a particular cardiac event in the respective cardiac cycle of the at least some of the cardiac cycles in the second sequence of consecutive cardiac cycles. In some embodiments, the second temporal relationship may be the same as the first temporal relationship. In some embodiments, for each respective cardiac cycle of the at least some of the cardiac cycles in the first sequence of consecutive cardiac cycles, the respective first particular time interval may be during a refractory period in the respective cardiac cycle of the at least some of the cardiac cycles in the first sequence of consecutive cardiac cycles, and for each respective cardiac cycle of the at least some of the cardiac cycles in the second sequence of consecutive cardiac cycles, the respective second particular time interval may be during a refractory period in the respective cardiac cycle of the at least some of the cardiac cycles in the second sequence of consecutive cardiac cycles.

In some embodiments, the data processing device system may be configured at least by the program at least to cause, in association with the first state, each of at least one first high voltage pulse of the plurality of first high voltage pulses to deliver a respective first amount of pulse energy. In some embodiments, the data processing device system may be configured at least by the program at least to cause, in association with the second state, each of at least one second high voltage pulse of the plurality of second high voltage pulses to deliver a respective second amount of pulse energy. In some embodiments, each respective second amount of pulse energy may be different than each respective first amount of pulse energy.

In some embodiments, the data processing device system may be configured at least by the program at least to cause, in association with the first state, each of at least one first high voltage pulse of the plurality of first high voltage pulses to have a respective first pulse shape. In some embodiments, the data processing device system may be configured at least by the program at least to cause, in association with the second state, each of at least one second high voltage pulse of the plurality of second high voltage pulses to have a respective second pulse shape. In some embodiments, each respective second pulse shape may be different than each respective first pulse shape.

In some embodiments, the first characteristic may indicate at least that the at least the particular cardiac cycle of the patient has a first duration. In some embodiments, the second characteristic may indicate at least that the at least the particular cardiac cycle of the patient has a second duration different than the first duration. In some embodiments, the second duration may be shorter than the first duration, and the first particular ratio of the total number of the first high voltage pulses to the total number of cardiac cycles in the first sequence of consecutive cardiac cycles may be greater than the second particular ratio of the total number of the second high voltage pulses to the total number of cardiac cycles in the second sequence of consecutive cardiac cycles.

In some embodiments, the first characteristic may indicate at least that each of the at least the particular cardiac cycle of the patient corresponds to a regular heartbeat, and the second characteristic may indicate at least that each of the at least the particular cardiac cycle of the patient corresponds to an irregular heartbeat. In some embodiments, the total number of first high voltage pulses in the plurality of first high voltage pulses may be the same as the total number of second high voltage pulses in the plurality of second high voltage pulses. In some embodiments, the total number of cardiac cycles in the first sequence of consecutive cardiac cycles may be different than the total number of cardiac cycles in the second sequence of consecutive cardiac cycles.

Various systems may include combinations and subsets of all the systems summarized above or otherwise described herein.

A pulsed field ablation system may be summarized as including, according to various embodiments, an input-output device system, a memory device system storing a program, and a data processing device system communicatively connected to the input-output device system and the memory device system. According to various embodiments, the data processing device system may be configured at least by the program at least to cause, via the input-output device system, each of at least a first transducer of a plurality of transducers located on a catheter device, to deliver a respective first high voltage pulse train of a first high voltage pulse train set during a first cardiac cycle, each high voltage pulse train of the first high voltage pulse train set configured to cause pulsed field ablation of tissue. In some embodiments, the data processing device system may be configured at least by the program at least to cause, via the input-output device system, of information indicative of temperature at least proximate a second transducer of the plurality of transducers during or after delivery of at least part of the first high voltage pulse train set. In some embodiments, the data processing device system may be configured at least by the program at least to determine, based at least on the information indicative of temperature at least proximate the second transducer, a particular pulse train parameter set of each respective second high voltage pulse train of a second high voltage pulse train set. In some embodiments, each high voltage pulse train of the second high voltage pulse train set may be configured to cause pulsed field ablation of tissue. In some embodiments, the particular pulse train parameter set of each respective second high voltage pulse train may include at least one pulse train parameter that is different than a corresponding pulse train parameter of a pulse train parameter set of the respective first high voltage pulse train delivered by the first transducer during the first cardiac cycle. In some embodiments, the data processing device system may be configured at least by the program at least to cause, via the input-output device system, each of at least a third transducer of the plurality of transducers, to deliver a respective second high voltage pulse train of the second high voltage pulse train set with the determined particular parameter set during a second cardiac cycle subsequent to the first cardiac cycle.

In some embodiments, the at least one pulse train parameter of the determined particular pulse train parameter set of each respective second high voltage pulse train may include a particular number of high voltage pulses in the respective second high voltage pulse train. In some embodiments, the at least one pulse train parameter of the determined particular pulse train parameter set of each respective second high voltage pulse train may include a particular pulse amplitude of each of one or more high voltage pulses in the second high voltage pulse train. In some embodiments, the at least one pulse train parameter of the determined particular pulse train parameter set of each respective second high voltage pulse train may include a particular pulse shape of each of one or more high voltage pulses in the second high voltage pulse train. In some embodiments, the at least one pulse train parameter of the determined particular pulse train parameter set of each respective second high voltage pulse train may include a particular inter-pulse spacing between high voltage pulses in the second high voltage pulse train. In some embodiments, the information indicative of temperature at least proximate the second transducer during, or after, delivery of at least part of the first high voltage pulse train set may indicate an increase in temperature, and the determined particular pulse train parameter set of the respective second high voltage pulse train delivered by the third transducer may be configured to cause the high voltage pulses of the respective second high voltage pulse train delivered by the third transducer to cumulatively deliver less energy during the second cardiac cycle than energy cumulatively delivered during the first cardiac cycle by the high voltage pulses of the respective first high voltage pulse train delivered by the first transducer. In some embodiments, the third transducer is the first transducer. In some embodiments, the second transducer is the first transducer. In some embodiments, each of the first transducer, the second transducer, and the third transducer is a pulsed field ablation transducer. In some embodiments, the information indicative of temperature at least proximate the second transducer is provided by the second transducer.

Various systems may include combinations and subsets of all the systems summarized above or otherwise described herein.

A pulsed field ablation system may be summarized as including, according to various embodiments, an input-output device system, a memory device system storing a program, and a data processing device system communicatively connected to the input-output device system and the memory device system. In some embodiments, the data processing device system may be configured at least by the program at least to cause, via the input-output device system, each of at least a first transducer of a plurality of transducers located on a catheter device, to deliver a respective first high voltage pulse train of a first high voltage pulse train set during a first cardiac cycle, each high voltage pulse train of the first high voltage pulse train set configured to cause pulsed field ablation of tissue. In some embodiments, the data processing device system may be configured at least by the program at least to cause reception, via the input-output device system, of information indicative of impedance at least proximate a second transducer of the plurality of transducers during or after delivery of at least part of the first high voltage pulse train set. In some embodiments, the data processing device system may be configured at least by the program at least to determine, based at least on the information indicative of impedance at least proximate the second transducer, a particular pulse train parameter set of each respective second high voltage pulse train of a second high voltage pulse train set. In some embodiments, each high voltage pulse train of the second high voltage pulse train set may be configured to cause pulsed field ablation of tissue. In some embodiments, the particular pulse train parameter set of each respective second high voltage pulse train may include at least one pulse train parameter that is different than a corresponding pulse train parameter of a pulse train parameter set of the respective first high voltage pulse delivered by the first transducer during the first cardiac cycle. In some embodiments, the data processing device system may be configured at least by the program at least to cause, via the input-output device system, each of at least a third transducer of the plurality of transducers, to deliver a respective second high voltage pulse train of the second high voltage pulse train set with the determined particular parameter set during a second cardiac cycle subsequent to the first cardiac cycle.

In some embodiments, the at least one pulse train parameter of the determined particular pulse train parameter set of each respective second high voltage pulse train may include a particular number of high voltage pulses in the respective second high voltage pulse train. In some embodiments, the at least one pulse train parameter of the determined particular pulse train parameter set of each respective second high voltage pulse train may include a particular pulse amplitude of each of one or more high voltage pulses in the second high voltage pulse train. In some embodiments, the at least one pulse train parameter of the determined particular pulse train parameter set of each respective second high voltage pulse train may include a particular pulse shape of each of one or more high voltage pulses in the second high voltage pulse train. In some embodiments, the at least one pulse train parameter of the determined particular pulse train parameter set of each respective second high voltage pulse train may include a particular inter-pulse spacing between high voltage pulses in the second high voltage pulse train. In some embodiments, the information indicative of impedance at least proximate the second transducer during, or after, delivery of at least part of the first high voltage pulse train set may indicate a decrease in impedance, and the determined particular pulse train parameter set of the respective second high voltage pulse train delivered by the third transducer may be configured to cause the high voltage pulses of the respective second high voltage pulse train delivered by the third transducer to cumulatively deliver less energy during the second cardiac cycle than energy cumulatively delivered during the first cardiac cycle by the high voltage pulses of the respective first high voltage pulse train delivered by the first transducer.

In some embodiments, the third transducer is the first transducer. In some embodiments, the second transducer is the first transducer. In some embodiments, each of the first transducer, the second transducer, and the third transducer is a pulsed field ablation transducer. In some embodiments, the information indicative of temperature at least proximate the second transducer is provided by the second transducer.

Various systems may include combinations and subsets of all the systems summarized above or otherwise described herein.

A pulsed field ablation system may be summarized as including, according to some embodiments, an input-output device system, a memory device system storing a program, and a data processing device system communicatively connected to the input-output device system and the memory device system. In some embodiments, the data processing device system may be configured at least by the program at least to cause, in association with a first state in which a particular cardiac cycle of a patient has a first duration, delivery, via the input-output device system and via a first pulsed field ablation transducer located on a catheter device, of a first high voltage pulse train during a first particular time interval. In some embodiments, a duration of the first particular time interval may be less than the first duration. In some embodiments, the first high voltage pulse train defines a first plurality of high voltage pulses, and the first high voltage pulse train may be configured to cause pulsed field ablation of tissue. In some embodiments, the data processing device system may be configured at least by the program at least to cause, in association with a second state in which the particular cardiac cycle of the patient has a second duration different than the first duration, delivery, via the input-output device system and via the first pulsed field ablation transducer, of a second high voltage pulse train during a second particular time interval. In some embodiments, a duration of the second particular time interval may be less than the second duration. In some embodiments, the second high voltage pulse train defining a second plurality of high voltage pulses, and the second high voltage pulse train may be configured to cause pulsed field ablation of tissue. In some embodiments, the second plurality of high voltage pulses of the second high voltage pulse train may have a different number of high voltage pulses than the first high voltage pulse train.

A pulsed field ablation system may be summarized as including, according to various embodiments, an input-output device system, a memory device system storing a program, and a data processing device system communicatively connected to the input-output device system and the memory device system. In some embodiments, the data processing device system may be configured at least by the program at least to cause, in association with a first state in which a first plurality of consecutive cardiac cycles of a patient exhibit a non-irregular heart rate, a first pulsed field ablation transducer located on a catheter device to deliver pulsed field ablation energy during each of some, but not all, of the first plurality of consecutive cardiac cycles, the some, but not all, of the first plurality of consecutive cardiac cycles excluding at least one cardiac cycle of the first plurality of consecutive cardiac cycles during which no pulsed field ablation energy is delivered by the first pulsed field ablation transducer, the excluded at least one cardiac cycle of the first plurality of consecutive cardiac cycles occurring between at least two cardiac cycles of the some, but not all, of the first plurality of consecutive cardiac cycles. In some embodiments, the non-irregular heart rate is a constant heart rate.

A pulsed field ablation system may be summarized as including, according to various embodiments, an input-output device system, a memory device system storing a program, and a data processing device system communicatively connected to the input-output device system and the memory device system. In some embodiments, the data processing device system may be configured at least by the program at least to identify a particular pulsed field ablation transducer set of a catheter device, the particular pulsed field ablation transducer set identified from a plurality of pulsed field ablation transducers of the catheter device. In some embodiments, the particular pulsed field ablation transducer set identified to be activated to apply a high voltage pulse train between the pulsed field ablation transducers of the particular pulsed field ablation transducer set, the high voltage pulse train sufficient to cause pulsed field ablation of tissue. In some embodiments, the data processing device system may be configured at least by the program at least to in association with a first state in which the identified particular pulsed field ablation transducer set is a first set of pulsed field ablation transducers of the catheter device, determine a first particular parameter set of the high voltage pulse train and cause activation, via the input-output device system, of the identified particular pulsed field ablation transducer set to deliver the high voltage pulse train in accordance with the determined first particular parameter set. In some embodiments, the data processing device system may be configured at least by the program at least to in association with a second state in which the identified particular pulsed field ablation transducer set is a second set of pulsed field ablation transducers of the catheter device different than the first set of pulsed field ablation transducers, determine a second particular parameter set of the high voltage pulse train different than the first particular parameter set and cause activation, via the input-output device system, of the identified particular pulsed field ablation transducer set to deliver the high voltage pulse train in accordance with the determined second particular parameter set.

In some embodiments, in the first state in which the identified particular pulsed field ablation transducer set is the first set of pulsed field ablation transducers of the catheter device, the first set of pulsed field ablation transducers has a first number of pulsed field ablation transducers, and in the second state in which the identified particular pulsed field ablation transducer set is the second set of pulsed field ablation transducers of the catheter device, the second set of pulsed field ablation transducers has a second number of pulsed field ablation transducers. In some embodiments, the second number of pulsed field ablation transducers may be greater than the first number of pulsed field ablation transducers. In some embodiments, each pulsed field ablation transducer of the identified particular pulsed field ablation transducer set includes a respective electrode, each respective electrode including a respective energy delivery surface configured to deliver pulsed field ablation energy. In some embodiments, in the first state in which the identified particular pulsed field ablation transducer set is the first set of pulsed field ablation transducers of the catheter device, each energy delivery surface of at least one energy delivery surface of the first set of pulsed field ablation transducers may have a first area, and in the second state in which the identified particular pulsed field ablation transducer set is the second set of pulsed field ablation transducers of the catheter device, each energy delivery surface of at least one energy delivery surface of the second set of pulsed field ablation transducers may have a second area different than the first area. In some embodiments, in the first state in which the identified particular pulsed field ablation transducer set is the first set of pulsed field ablation transducers of the catheter device, the energy delivery surface of each of at least one pulsed field ablation transducer of the first set of pulsed field ablation transducers has a first area, and in the second state in which the identified particular pulsed field ablation transducer set is the second set of pulsed field ablation transducers of the catheter device, the energy delivery surfaces of each of at least one pulsed filed ablation transducer of the second set of pulsed field ablation transducers may have a second area the same as the first area. In some embodiments, each energy delivery surface of the first set of pulsed field ablation transducers in the first state may have a different area than each energy delivery surface of the second set of pulsed field ablation transducers in the second state. In some embodiments, each pulsed field ablation transducer of the identified particular pulsed field ablation transducer set includes a respective electrode, each respective electrode including a respective energy delivery surface configured to deliver pulsed field ablation energy. In some embodiments, (a) in the first state in which the identified particular pulsed field ablation transducer set is the first set of pulsed field ablation transducers of the catheter device, the energy delivery surfaces of the first set of pulsed field ablation transducers may have a same area, or (b) in the second state in which the identified particular pulsed field ablation transducer set is the second set of transducers of the catheter device, the energy delivery surfaces of the second set of transducers may have a same area. In some embodiments, each pulsed field ablation transducer of the identified particular pulsed field ablation transducer set includes a respective electrode, each respective electrode including a respective energy delivery surface configured to deliver pulsed field ablation energy. In some embodiments, (c) in the first state in which the identified particular pulsed field ablation transducer set is the first set of pulsed field ablation transducers of the catheter device, the energy delivery surfaces of the first set of pulsed field ablation transducers may have a same geometric shape, or (d) in the second state in which the identified particular pulsed field ablation transducer set is the second set of pulsed field ablation transducers of the catheter device, the energy delivery surfaces of the second set of pulsed field ablation transducers may have a same geometric shape.

In some embodiments, each pulsed field ablation transducer of the identified particular pulsed field ablation transducer set includes a respective electrode, each respective electrode including a respective energy delivery surface configured to deliver pulsed field ablation energy. In some embodiments, in the first state in which the identified particular pulsed field ablation transducer set is the first set of pulsed field ablation transducers of the catheter device, the energy delivery surfaces of the first set of pulsed field ablation transducers have a first collective area, and in the second state in which the identified particular pulsed field ablation transducer set is the second set of pulsed field ablation transducers of the catheter device, the energy delivery surfaces of the second set of pulsed field ablation transducers have a second collective area. In some embodiments, the second collective area may be greater than the first collective area.

In some embodiments, each pulsed field ablation transducer of the identified particular pulsed field ablation transducer set may include a respective electrode, each respective electrode including a respective energy delivery surface configured to deliver pulsed field ablation energy. In some embodiments, in the first state in which the identified particular pulsed field ablation transducer set is the first set of pulsed field ablation transducers of the catheter device, the energy delivery surfaces of the first set of pulsed field ablation transducers have a first set of one or more geometric shapes, and in the second state in which the identified particular pulsed field ablation transducer set is the second set of pulsed field ablation transducers of the catheter device. In some embodiments, the energy delivery surfaces of the second set of pulsed field ablation transducers may have a second set of one or more geometric shapes different than the first set of one or more geometric shapes.

In some embodiments, the particular pulsed field ablation transducer set may be identified based at least on a selection of at least two pulsed field ablation transducers of the catheter device, each pulsed field ablation transducer of the at least two pulsed field ablation transducers configured to selectively deliver energy sufficient for pulsed field ablation of tissue. In some embodiments, the selection of the at least two pulsed field ablation transducers of the catheter device may be a user selection of the at least two pulsed field ablation transducers.

In some embodiments, the data processing device system may be configured at least by the program at least to perform an analysis of a total number of at least the pulsed field ablation transducers in the particular pulsed field ablation transducer set. In some embodiments, in the first state, the analysis of the total number of the at least the pulsed field ablation transducers in the particular pulsed field ablation transducer set may be an analysis of a total number of pulsed field ablation transducers in the first set of pulsed field ablation transducers. In some embodiments, in the second state, the analysis of the total number of the at least the pulsed field ablation transducers in the particular pulsed field ablation transducer set may be an analysis of a total number of pulsed field ablation transducers in the second set of pulsed field ablation transducers. In some embodiments, in the first state, the first particular parameter set of the high voltage pulse train may be determined based at least on the analysis of the total number of pulsed field ablation transducers in the first set of pulsed field ablation transducers, and in the second state, the second particular parameter set of the high voltage pulse train may be determined based at least on the analysis of the total number of pulsed field ablation transducers in the second set of pulsed field ablation transducers.

In some embodiments, the data processing device system may be configured at least by the program at least to perform an analysis of a transducer type of each pulsed field ablation transducer of at least the pulsed field ablation transducers of the particular pulsed field ablation transducer set. In some embodiments, in the first state, the analysis of a transducer type of each pulsed field ablation transducer in the at least the pulsed field ablation transducers in the particular pulsed field ablation transducer set may be an analysis of a transducer type of each pulsed field ablation transducer in the first set of pulsed field ablation transducers. In some embodiments, in the second state, the analysis of a transducer type of each pulsed field ablation transducer in the at least the pulsed field ablation transducers in the particular pulsed field ablation transducer set may be an analysis of a transducer type of each pulsed field ablation transducer in the second set of pulsed field ablation transducers. In some embodiments, in the first state, the first particular parameter set of the high voltage pulse train may be determined based at least on the analysis of a transducer type of each pulsed field ablation transducer in the first set of pulsed field ablation transducers, and in the second state, the second particular parameter set of the high voltage pulse train may be determined based at least on the analysis of a transducer type of each pulsed field ablation transducer in the second set of pulsed field ablation transducers.

In some embodiments, the data processing device system may be configured at least by the program at least to perform an analysis of size, shape, or size and shape of each pulsed field ablation transducer of at least the pulsed field ablation transducers of the particular pulsed field ablation transducer set. In some embodiments, in the first state, the analysis of size, shape, or size and shape of each pulsed field ablation transducer in the at least the pulsed field ablation transducers in the particular pulsed field ablation transducer set may be an analysis of size, shape, or size and shape of each pulsed field ablation transducer in the first set of pulsed field ablation transducers. In some embodiments, in the second state, the analysis of size, shape, or size and shape of each pulsed field ablation transducer in the at least the pulsed field ablation transducers in the particular pulsed field ablation transducer set may be an analysis of size, shape, or size and shape of each pulsed field ablation transducer in the second set of pulsed field ablation transducers. In some embodiments, in the first state, the first particular parameter set of the high voltage pulse train may be determined based at least on the analysis of size, shape, or size and shape of each pulsed field ablation transducer in the first set of pulsed field ablation transducers, and in the second state, the second particular parameter set of the high voltage pulse train may be determined based at least on the analysis of size, shape, or size and shape of each pulsed field ablation transducer in the second set of pulsed field ablation transducers.

In some embodiments, the particular pulsed field ablation transducer set may be identified based at least on an analysis of degree of tissue contact exhibited by at least the pulsed field ablation transducers of the particular pulsed field ablation transducer set. In some embodiments, the particular pulsed field ablation transducer set may be identified based at least on an analysis of data provided by each pulsed field ablation transducer of at least the pulsed field ablation transducers of the particular pulsed field ablation transducer set.

In some embodiments, each pulsed field ablation transducer of the identified particular pulsed field ablation transducer set includes a respective electrode, each respective electrode including a respective energy delivery surface configured to deliver pulsed field ablation energy. In some embodiments, in the first state in which the identified particular pulsed field ablation transducer set is the first set of pulsed field ablation transducers of the catheter device, each energy delivery surface of at least one energy delivery surface of the first set of pulsed field ablation transducers has a first geometric shape, and in the second state in which the identified particular pulsed field ablation transducer set is the second set of pulsed field ablation transducers of the catheter device, each energy delivery surface of at least one energy delivery surface of the second set of pulsed field ablation transducers has a second geometric shape. In some embodiments, the second geometric shape may be different than the first geometric shape. In some embodiments, the respective energy delivery surfaces of the first set of pulsed field ablation transducers in the first state may have a same area. In some embodiments, the respective energy delivery surfaces of the second set of transducers in the second state may have a same area.

In some embodiments, each high voltage pulse in the high voltage pulse train is configured to deliver a respective amount of pulse energy, and wherein the pulse energy deliverable by each of at least one high voltage pulse in the high voltage pulse train in accordance with the second particular parameter set may be less than the pulse energy deliverable by each of at least one high voltage pulse in the high voltage pulse train in accordance with the first particular parameter set.

In some embodiments, each high voltage pulse in the high voltage pulse train includes a respective rise time, and the respective rise time of each high voltage pulse of the high voltage pulse train in accordance with the second particular parameter set may be longer than the respective rise time of each high voltage pulse of the high voltage pulse train in accordance with the first particular parameter set.

In some embodiments, each of the first particular parameter set and the second particular parameter set defines a respective pulse duration of each of at least one high voltage pulse in the high voltage pulse train, and the respective pulse duration of each of the at least one high voltage pulse in the high voltage pulse train defined in accordance with the second particular parameter set may be less than the respective pulse duration of each of the at least one high voltage pulse in the high voltage pulse train defined in accordance with the first particular parameter set.

In some embodiments, each of the first particular parameter set and the second particular parameter set defines a respective pulse frequency of the pulses in the high voltage pulse train, and the respective pulse frequency of the pulses in the high voltage pulse train defined in accordance with the second particular parameter set may be lower than the respective pulse frequency of the pulses in the high voltage pulse train defined in accordance with the first particular parameter set.

In some embodiments, each of the first particular parameter set and the second particular parameter set defines a respective number of pulses in the high voltage pulse train, and the respective number of pulses in the high voltage pulse train defined in accordance with the second particular parameter set may be less than the respective number of pulses in the high voltage pulse train defined in accordance with the first particular parameter set.

In some embodiments, the data processing device system may be configured at least by the program at least to cause the high voltage pulse train to deliver, in the first state, a first average power in accordance with the first particular parameter set, and cause the high voltage pulse train to deliver, in the second state, a second average power in accordance with the second particular parameter set. In some embodiments, the second average power may be within 10% of the first average power.

In some embodiments, the high voltage pulse train is a first high voltage pulse train of a plurality of high voltage pulse trains, and the data processing device system may be configured at least by the program at least to cause activation, via the input-output device system, of the particular pulsed field ablation transducer set to deliver each high voltage pulse train of the plurality of high voltage pulse trains during a respective cardiac cycle of a plurality of cardiac cycles.

In some embodiments, the determination of the first particular parameter set may include a delivery of a first preliminary or test signal set between the pulsed field ablation transducers in the first set of pulsed field ablation transducers. In some embodiments, the determination of the second particular parameter set may include a delivery of a second preliminary or test signal set between the pulsed field ablation transducers in the first set of pulsed field ablation transducers.

Various systems may include combinations and subsets of all the systems summarized above or otherwise described herein.

A pulsed field ablation system may be summarized as including, according to some embodiments, an input-output device system, a memory device system storing a program, and a data processing device system communicatively connected to the input-output device system and the memory device system. In some embodiments, the data processing device system may be configured at least by the program at least to cause detection, via the input-output device system, of a degree of tissue contact exhibited by a portion of a catheter device. In some embodiments, the data processing device system may be configured at least by the program at least to cause activation, via the input-output device system, of a particular pulsed field ablation transducer set to deliver a high voltage pulse train, the high voltage pulse train sufficient to cause pulsed field ablation of tissue. In some embodiments, the data processing device system may be configured at least by the program at least to, in response to a first state in which the detected degree of tissue contact is a first degree, determine a first particular parameter set of at least the high voltage pulse train and cause the activation, via the input-output device system, of the particular pulsed field ablation transducer set to deliver the high voltage pulse train in accordance with the determined first particular parameter set. In some embodiments, the data processing device system may be configured at least by the program at least to, in response to a second state in which the detected degree of tissue contact is a second degree, determine a second particular parameter set of at least the high voltage pulse train different than the first particular parameter set. In some embodiments, the data processing device system may be configured at least by the program at least to cause the activation, via the input-output device system, of the particular pulsed field ablation transducer set to deliver the high voltage pulse train in accordance with the determined second particular parameter set. In some embodiments, the first degree may indicate lesser tissue contact than the second degree, the high voltage pulses of the high voltage pulse train delivered in accordance with the first particular parameter set collectively deliver first energy, the high voltage pulses of the high voltage pulse train delivered in accordance with the second particular parameter set collectively deliver second energy, and the first energy is greater than the second energy.

In some embodiments, each of the first particular parameter set and the second particular parameter set defines a respective pulse frequency of the pulses in the high voltage pulse train, and the respective pulse frequency of the pulses in the high voltage pulse train defined in accordance with the first particular parameter set may be greater than the respective pulse frequency of the pulses in the high voltage pulse train defined in accordance with the second particular parameter set.

In some embodiments, each of the first particular parameter set and the second particular parameter set defines a respective number of pulses in the high voltage pulse train, and the respective number of pulses in the high voltage pulse train defined in accordance with the first particular parameter set may be greater than the respective number of pulses in the high voltage pulse train defined in accordance with the second particular parameter set.

In some embodiments, each of the first particular parameter set and the second particular parameter set defines a respective pulse duration of each of at least one high voltage pulse in the high voltage pulse train, and the respective pulse duration of each of the at least one high voltage pulse in the high voltage pulse train defined in accordance with the first particular parameter set may be greater than the respective pulse duration of each of the at least one high voltage pulse in the high voltage pulse train defined in accordance with the second particular parameter set.

In some embodiments, each of the first particular parameter set and the second particular parameter set defines a respective pulse duration of each high voltage pulse in the high voltage pulse train, and the respective pulse duration of each high voltage pulse in the high voltage pulse train defined in accordance with the first particular parameter set may be greater than the respective pulse duration of each high voltage pulse in the high voltage pulse train defined in accordance with the second particular parameter set.

In some embodiments, each of the first particular parameter set and the second particular parameter set defines a respective pulse amplitude of each of at least one high voltage pulse in the high voltage pulse train, and the respective pulse amplitude of each of the at least one high voltage pulse in the high voltage pulse train defined in accordance with the first particular parameter set may be greater than the respective pulse amplitude of each of the at least one high voltage pulse in the high voltage pulse train defined in accordance with the second particular parameter set.

In some embodiments, each of the first particular parameter set and the second particular parameter set defines a respective pulse amplitude of each high voltage pulse in the high voltage pulse train, and wherein the respective pulse amplitude of each high voltage pulse in the high voltage pulse train defined in accordance with the first particular parameter set may be greater than the respective pulse amplitude of each high voltage pulse in the high voltage pulse train defined in accordance with the second particular parameter set.

In some embodiments, each high voltage pulse in the high voltage pulse train is configured to deliver a respective amount of pulse energy, and wherein the pulse energy deliverable by each of at least one high voltage pulse in the high voltage pulse train in accordance with the first particular parameter set may be greater than the pulse energy deliverable by each of at least one high voltage pulse in the high voltage pulse train in accordance with the second particular parameter set.

In some embodiments, each high voltage pulse in the high voltage pulse train is configured to deliver a respective amount of pulse energy, and the pulse energy deliverable by each high voltage pulse in the high voltage pulse train in accordance with the first particular parameter set may be greater than the pulse energy deliverable by each high voltage pulse in the high voltage pulse train in accordance with the second particular parameter set.

In some embodiments, the portion of the catheter device may be provided by one or more transducers of the catheter device, the one or more transducers configured to be positioned within a body of a patient.

In some embodiments, the data processing device system may be configured to cause the detection, via the input-output device system, of the degree of tissue contact exhibited by the portion of the catheter device at least in part from a signal set provided by one or more transducers, the one or more transducers configured to be positioned within a body of a patient. In some embodiments, the one or more transducers may be provided by the catheter device. In some embodiments, the portion of the catheter device may be provided by one or more pulsed field ablation transducers of the catheter device. In some embodiments, the particular pulsed field ablation transducer set may include the one or more pulsed field ablation transducers of the catheter device.

In some embodiments, each pulsed field ablation transducer of the catheter device includes a respective electrode, each respective electrode including a respective energy delivery surface configured to deliver pulsed field ablation energy. In some embodiments, the data processing device system may be configured to cause the detection, via the input-output device system, of the degree of tissue contact exhibited by the portion of the catheter device at least by causing detection, via the input-output device system, of a degree of tissue contact exhibited by at least a part of the respective energy delivery surface of each of at least some of the pulsed field ablation transducers of the catheter device.

In some embodiments, the high voltage pulse train is a first high voltage pulse train of a plurality of high voltage pulse trains. In some embodiments, the data processing device system may be configured at least by the program at least to cause activation, via the input-output device system, of the particular pulsed field ablation transducer set to deliver each high voltage pulse train of the plurality of high voltage pulse trains during a respective cardiac cycle of a plurality of cardiac cycles. In some embodiments, in response to the first state in which the detected degree of tissue contact is the first degree, the data processing device system may be configured at least by the program at least to cause the activation, via the input-output device system, of the particular pulsed field ablation transducer set to deliver each high voltage pulse train of the plurality of high voltage pulse trains during the respective cardiac cycle of a plurality of cardiac cycles in accordance with the first particular parameter set. In some embodiments, in response to the second state in which the detected degree of tissue contact is the second degree, the data processing device system may be configured at least by the program at least to cause the activation, via the input-output device system, of the particular pulsed field ablation transducer set to deliver each high voltage pulse train of the plurality of high voltage pulse trains during the respective cardiac cycle of a plurality of cardiac cycles in accordance with the second particular parameter set.

Various systems may include combinations and subsets of all the systems summarized above or otherwise described herein.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

For example, in some embodiments, a pulsed field ablation method is executed by a data processing device system communicatively connected to an input-output device system, the method including causing delivery, via the input-output device system and via a first pulsed field ablation transducer located on a catheter device, of a respective high voltage pulse train during each respective cardiac cycle of a plurality of cardiac cycles including at least a first cardiac cycle and a second cardiac cycle. Each respective high voltage pulse train may define a plurality of high voltage pulses, and each respective high voltage pulse train may be configured to cause pulsed field ablation of tissue. Each respective high voltage pulse train may be caused to be delivered only during a particular time interval in the respective cardiac cycle, and the particular time intervals in the first cardiac cycle and the second cardiac cycle may be configured such that a first ratio of the duration of the particular time interval in the first cardiac cycle to the duration of the first cardiac cycle is different than a second ratio of the duration of the particular time interval in the second cardiac cycle to the duration of the second cardiac cycle. The high voltage pulses of the respective high voltage pulse train which are delivered during the first cardiac cycle may be configured to cumulatively deliver first energy during the particular time interval in the first cardiac cycle, and the high voltage pulses of the respective high voltage pulse train which are delivered during the second cardiac cycle may be configured to cumulatively deliver second energy during the particular time interval in the second cardiac cycle. The second energy may be different than the first energy.

In some embodiments, a pulsed field ablation method is executed by a data processing device system communicatively connected to an input-output device system, the method including causing delivery, via the input-output device system and via a first pulsed field ablation transducer located on a catheter device, of a respective high voltage pulse train during each respective cardiac cycle of a plurality of cardiac cycles. Each respective high voltage pulse train may define a plurality of high voltage pulses, and each respective high voltage pulse train may be configured to cause pulsed field ablation of tissue. Each respective high voltage pulse train may be caused to be deliverable only during a particular time interval in the respective cardiac cycle. The method may include causing, in response to reception of information indicative of a particular cardiac event occurring in each of at least some of the plurality of cardiac cycles, a change in at least one high voltage pulse train parameter to cause at least two of the respective high voltage pulse trains to be different from each other.

In some embodiments, a pulsed field ablation method is executed by a data processing device system communicatively connected to an input-output device system, the method including causing, in association with a first state in which at least a particular cardiac cycle of a patient is determined to have a first duration, delivery, via the input-output device system and via a first pulsed field ablation transducer located on a catheter device, of a first high voltage pulse train during a first particular time interval. A duration of the first particular time interval may be less than the first duration, the first high voltage pulse train may define a first plurality of high voltage pulses, the first high voltage pulse train may be configured to cause pulsed field ablation of tissue, and the first plurality of high voltage pulses may be configured to cumulatively deliver first energy during the first particular time interval. The method may include causing, in association with a second state in which at least the particular cardiac cycle of the patient is determined to have a second duration different than the first duration, delivery, via the input-output device system and via the first pulsed field ablation transducer, of a second high voltage pulse train during a second particular time interval. A duration of the second particular time interval may be less than the second duration, the second high voltage pulse train may define a second plurality of high voltage pulses, the second high voltage pulse train may be configured to cause pulsed field ablation of tissue, and the second plurality of high voltage pulses may be configured to cumulatively deliver second energy during the second particular time interval, the second energy different than the first energy.

In some embodiments, a pulsed field ablation method is executed by a data processing device system, the method including causing, in association with a first state in which at least a particular cardiac cycle of a patient has a first characteristic, a first pulsed field ablation transducer located on a catheter device to deliver a plurality of first high voltage pulses during a first sequence of consecutive cardiac cycles. The plurality of first high voltage pulses may be configured to deliver a particular average power throughout a duration of the first sequence of consecutive cardiac cycles. The method may include causing, in association with a second state in which at least the particular cardiac cycle of a patient has a second characteristic different than the first characteristic, the first pulsed field ablation transducer to deliver a plurality of second high voltage pulses during a second sequence of consecutive cardiac cycles. The delivery of the plurality of second high voltage pulses during the second sequence of consecutive cardiac cycles in association with the second state may be configured to maintain the particular average power delivered by the first pulsed field ablation transducer throughout the duration of the first sequence of consecutive cardiac cycles in association with the first state. Each of the plurality of first high voltage pulses and the plurality of second high voltage pulses may be configured to cause pulsed field ablation of tissue. A first particular ratio of a total number of the first high voltage pulses to a total number of cardiac cycles in the first sequence of consecutive cardiac cycles may be different than a second particular ratio of a total number of the second high voltage pulses to a total number of cardiac cycles in the second sequence of consecutive cardiac cycles.

In some embodiments, a pulsed field ablation method may be executed by a data processing device system communicatively connected to an input-output device system, the method including causing, via the input-output device system, each of at least a first transducer of a plurality of transducers located on a catheter device to deliver a respective first high voltage pulse train of a first high voltage pulse train set during a first cardiac cycle. Each high voltage pulse train of the first high voltage pulse train set may be configured to cause pulsed field ablation of tissue. The method may include receiving, via the input-output device system, information indicative of temperature at least proximate a second transducer of the plurality of transducers during or after delivery of at least part of the first high voltage pulse train set. The method may include determining, based at least on the information indicative of temperature at least proximate the second transducer, of a particular pulse train parameter set of each respective second high voltage pulse train of a second high voltage pulse train set. Each high voltage pulse train of the second high voltage pulse train set may be configured to cause pulsed field ablation of tissue, and the particular pulse train parameter set of each respective second high voltage pulse train may include at least one pulse train parameter that is different than a corresponding pulse train parameter of a pulse train parameter set of the respective first high voltage pulse delivered by the first transducer during the first cardiac cycle. The method may include causing, via the input-output device system, each of at least a third transducer of the plurality of transducers to deliver a respective second high voltage pulse train of the second high voltage pulse train set with the determined particular parameter set during a second cardiac cycle subsequent to the first cardiac cycle.

In some embodiments, a pulsed field ablation method is executed by a data processing device system communicatively connected to an input-output device system, the method including causing, via the input-output device system, each of at least a first transducer of a plurality of transducers located on a catheter device to deliver a respective first high voltage pulse train of a first high voltage pulse train set during a first cardiac cycle. Each high voltage pulse train of the first high voltage pulse train set may be configured to cause pulsed field ablation of tissue. The method may include receiving, via the input-output device system, information indicative of impedance at least proximate a second transducer of the plurality of transducers during or after delivery of at least part of the first high voltage pulse train set. The method may include determining, based at least on the information indicative of impedance at least proximate the second transducer, a particular pulse train parameter set of each respective second high voltage pulse train of a second high voltage pulse train set. Each high voltage pulse train of the second high voltage pulse train set may be configured to cause pulsed field ablation of tissue, and the particular pulse train parameter set of each respective second high voltage pulse train may include at least one pulse train parameter that is different than a corresponding pulse train parameter of a pulse train parameter set of the respective first high voltage pulse train delivered by the first transducer during the first cardiac cycle. The method may include causing, via the input-output device system, each of at least a third transducer of the plurality of transducers to deliver a respective second high voltage pulse train of the second high voltage pulse train set with the determined particular parameter set during a second cardiac cycle subsequent to the first cardiac cycle.

In some embodiments, a pulsed field ablation method is executed by a data processing device system communicatively connected to an input-output device system, the method including causing, in association with a first state in which a particular cardiac cycle of a patient has a first duration, delivery, via the input-output device system and via a first pulsed field ablation transducer located on a catheter device, of a first high voltage pulse train during a first particular time interval. A duration of the first particular time interval may be less than the first duration, the first high voltage pulse train may define a first plurality of high voltage pulses, and the first high voltage pulse train may be configured to cause pulsed field ablation of tissue. The method may include causing, in association with a second state in which the particular cardiac cycle of the patient has a second duration different than the first duration, delivery, via the input-output device system and via the first pulsed field ablation transducer, of a second high voltage pulse train during a second particular time interval. A duration of the second particular time interval may be less than the second duration, the second high voltage pulse train may define a second plurality of high voltage pulses, the second high voltage pulse train may be configured to cause pulsed field ablation of tissue, and the second plurality of high voltage pulses of the second high voltage pulse train may have a different number of high voltage pulses than the first high voltage pulse train.

In some embodiments, a pulsed field ablation method is executed by a data processing device system, the method including causing, in association with a first state in which a first plurality of consecutive cardiac cycles of a patient exhibit a non-irregular heart rate, a first pulsed field ablation transducer located on a catheter device to deliver pulsed field ablation energy during each of some, but not all, of the first plurality of consecutive cardiac cycles. The some, but not all, of the first plurality of consecutive cardiac cycles may exclude at least one cardiac cycle of the first plurality of consecutive cardiac cycles during which no pulsed field ablation energy is delivered by the first pulsed field ablation transducer. Thee excluded at least one cardiac cycle of the first plurality of consecutive cardiac cycles may occur between at least two cardiac cycles of the some, but not all, of the first plurality of consecutive cardiac cycles.

In some embodiments, a pulsed field ablation method is executed by a data processing device system communicatively connected to an input-output device system, the method including identifying a particular pulsed field ablation transducer set of a catheter device. The particular pulsed field ablation transducer set may be identified from a plurality of pulsed field ablation transducers of the catheter device, and the particular pulsed field ablation transducer set may be identified to be activated to apply a high voltage pulse train between the pulsed field ablation transducers of the particular pulsed field ablation transducer set. The high voltage pulse train may be sufficient to cause pulsed field ablation of tissue. The method may include, in association with a first state in which the identified particular pulsed field ablation transducer set is a first set of pulsed field ablation transducers of the catheter device, determining a first particular parameter set of the high voltage pulse train and causing activation, via the input-output device system, of the identified particular pulsed field ablation transducer set to deliver the high voltage pulse train in accordance with the determined first particular parameter set. The method may include, in association with a second state in which the identified particular pulsed field ablation transducer set is a second set of pulsed field ablation transducers of the catheter device different than the first set of pulsed field ablation transducers, determining a second particular parameter set of the high voltage pulse train different than the first particular parameter set and causing activation, via the input-output device system, of the identified particular pulsed field ablation transducer set to deliver the high voltage pulse train in accordance with the determined second particular parameter set.

In some embodiments, a pulsed field ablation method is executed by a data processing device system communicatively connected to an input-output device system, the method including causing detection, via the input-output device system, of a degree of tissue contact exhibited by a portion of a catheter device. The method may include causing activation, via the input-output device system, of a particular pulsed field ablation transducer set to deliver a high voltage pulse train. The high voltage pulse train may be sufficient to cause pulsed field ablation of tissue. The method may include, in response to a first state in which the detected degree of tissue contact is a first degree, determining a first particular parameter set of at least the high voltage pulse train and causing the activation, via the input-output device system, of the particular pulsed field ablation transducer set to deliver the high voltage pulse train in accordance with the determined first particular parameter set. The method may include, in response to a second state in which the detected degree of tissue contact is a second degree, determining a second particular parameter set of at least the high voltage pulse train different than the first particular parameter set and causing the activation, via the input-output device system, of the particular pulsed field ablation transducer set to deliver the high voltage pulse train in accordance with the determined second particular parameter set. The first degree may indicate lesser tissue contact than the second degree. The high voltage pulses of the high voltage pulse train delivered in accordance with the first particular parameter set may collectively deliver first energy, the high voltage pulses of the high voltage pulse train delivered in accordance with the second particular parameter set may collectively deliver second energy, and the first energy may be greater than the second energy.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

For example, in some embodiments, a computer-readable data storage medium system includes one or more computer-readable data storage mediums storing a program executable by one or more data processing devices of a data processing device system of a pulsed field ablation system, the program including first delivery instructions configured to cause delivery, via a first pulsed field ablation transducer located on a catheter device, of a respective high voltage pulse train during each respective cardiac cycle of a plurality of cardiac cycles including at least a first cardiac cycle and a second cardiac cycle. Each respective high voltage pulse train may define a plurality of high voltage pulses, and each respective high voltage pulse train may be configured to cause pulsed field ablation of tissue. The program may include second delivery instructions configured to cause delivery of each respective high voltage pulse train only during a particular time interval in the respective cardiac cycle. The particular time intervals in the first cardiac cycle and the second cardiac cycle may be configured such that a first ratio of the duration of the particular time interval in the first cardiac cycle to the duration of the first cardiac cycle is different than a second ratio of the duration of the particular time interval in the second cardiac cycle to the duration of the second cardiac cycle. The high voltage pulses of the respective high voltage pulse train which are delivered during the first cardiac cycle may be configured to cumulatively deliver first energy during the particular time interval in the first cardiac cycle, and the high voltage pulses of the respective high voltage pulse train which are delivered during the second cardiac cycle may be configured to cumulatively deliver second energy during the particular time interval in the second cardiac cycle. The second energy may be different than the first energy.

In some embodiments, a computer-readable data storage medium system includes one or more computer-readable data storage mediums storing a program executable by one or more data processing devices of a data processing device system of a pulsed field ablation system, the program including delivery instructions configured to cause delivery, via a first pulsed field ablation transducer located on a catheter device, of a respective high voltage pulse train during each respective cardiac cycle of a plurality of cardiac cycles. Each respective high voltage pulse train may define a plurality of high voltage pulses, and each respective high voltage pulse train may be configured to cause pulsed field ablation of tissue. The delivery instructions may be configured to cause each respective high voltage pulse train to be deliverable only during a particular time interval in the respective cardiac cycle. The program may include change instructions configured to cause, in response to reception of information indicative of a particular cardiac event occurring in each of at least some of the plurality of cardiac cycles, a change in at least one high voltage pulse train parameter to cause at least two of the respective high voltage pulse trains to be different from each other.

In some embodiments, a computer-readable data storage medium system includes one or more computer-readable data storage mediums storing a program executable by one or more data processing devices of a data processing device system of a pulsed field ablation system, the program including first delivery instructions configured to cause, in association with a first state in which at least a particular cardiac cycle of a patient is determined to have a first duration, delivery, via a first pulsed field ablation transducer located on a catheter device, of a first high voltage pulse train during a first particular time interval. A duration of the first particular time interval may be less than the first duration, and the first high voltage pulse train may define a first plurality of high voltage pulses. The first high voltage pulse train may be configured to cause pulsed field ablation of tissue, and the first plurality of high voltage pulses may be configured to cumulatively deliver first energy during the first particular time interval. The program may include second delivery instructions configured to cause, in association with a second state in which at least the particular cardiac cycle of the patient is determined to have a second duration different than the first duration, delivery, via the first pulsed field ablation transducer, of a second high voltage pulse train during a second particular time interval. A duration of the second particular time interval may be less than the second duration. The second high voltage pulse train may define a second plurality of high voltage pulses. The second high voltage pulse train may be configured to cause pulsed field ablation of tissue. The second plurality of high voltage pulses may be configured to cumulatively deliver second energy during the second particular time interval. The second energy may be different than the first energy.

In some embodiments, a computer-readable data storage medium system includes one or more computer-readable data storage mediums storing a program executable by one or more data processing devices of a data processing device system of a pulsed field ablation system, the program including first delivery instructions configured to cause, in association with a first state in which at least a particular cardiac cycle of a patient has a first characteristic, a first pulsed field ablation transducer located on a catheter device to deliver a plurality of first high voltage pulses during a first sequence of consecutive cardiac cycles. Thee plurality of first high voltage pulses may be configured to deliver a particular average power throughout a duration of the first sequence of consecutive cardiac cycles. The program may include second delivery instructions configured to cause, in association with a second state in which at least the particular cardiac cycle of a patient has a second characteristic different than the first characteristic, the first pulsed field ablation transducer to deliver a plurality of second high voltage pulses during a second sequence of consecutive cardiac cycles. The delivery of the plurality of second high voltage pulses during the second sequence of consecutive cardiac cycles in association with the second state may be configured to maintain the particular average power delivered by the first pulsed field ablation transducer throughout the duration of the first sequence of consecutive cardiac cycles in association with the first state. Each of the plurality of first high voltage pulses and the plurality of second high voltage pulses may be configured to cause pulsed field ablation of tissue. A first particular ratio of a total number of the first high voltage pulses to a total number of cardiac cycles in the first sequence of consecutive cardiac cycles may be different than a second particular ratio of a total number of the second high voltage pulses to a total number of cardiac cycles in the second sequence of consecutive cardiac cycles.

In some embodiments, a computer-readable data storage medium system includes one or more computer-readable data storage mediums storing a program executable by one or more data processing devices of a data processing device system of a pulsed field ablation system, the program including first delivery instructions configured to cause each of at least a first transducer of a plurality of transducers located on a catheter device to deliver a respective first high voltage pulse train of a first high voltage pulse train set during a first cardiac cycle. Each high voltage pulse train of the first high voltage pulse train set may be configured to cause pulsed field ablation of tissue. The program may include reception instructions configured to cause reception of information indicative of temperature at least proximate a second transducer of the plurality of transducers during or after delivery of at least part of the first high voltage pulse train set. The program may include determination instructions configured to cause determination, based at least on the information indicative of temperature at least proximate the second transducer, of a particular pulse train parameter set of each respective second high voltage pulse train of a second high voltage pulse train set. Each high voltage pulse train of the second high voltage pulse train set may be configured to cause pulsed field ablation of tissue, and the particular pulse train parameter set of each respective second high voltage pulse train may include at least one pulse train parameter that is different than a corresponding pulse train parameter of a pulse train parameter set of the respective first high voltage pulse delivered by the first transducer during the first cardiac cycle. The program may include second delivery instructions configured to cause each of at least a third transducer of the plurality of transducers to deliver a respective second high voltage pulse train of the second high voltage pulse train set with the determined particular parameter set during a second cardiac cycle subsequent to the first cardiac cycle.

In some embodiments, a computer-readable data storage medium system includes one or more computer-readable data storage mediums storing a program executable by one or more data processing devices of a data processing device system of a pulsed field ablation system, the program including first delivery instructions configured to cause each of at least a first transducer of a plurality of transducers located on a catheter device to deliver a respective first high voltage pulse train of a first high voltage pulse train set during a first cardiac cycle. Each high voltage pulse train of the first high voltage pulse train set may be configured to cause pulsed field ablation of tissue. The program may include reception instructions configured to cause reception of information indicative of impedance at least proximate a second transducer of the plurality of transducers during or after delivery of at least part of the first high voltage pulse train set. The program may include determination instructions configured to cause determination, based at least on the information indicative of impedance at least proximate the second transducer, of a particular pulse train parameter set of each respective second high voltage pulse train of a second high voltage pulse train set. Each high voltage pulse train of the second high voltage pulse train set may be configured to cause pulsed field ablation of tissue, and the particular pulse train parameter set of each respective second high voltage pulse train may include at least one pulse train parameter that is different than a corresponding pulse train parameter of a pulse train parameter set of the respective first high voltage pulse train delivered by the first transducer during the first cardiac cycle. The program may include second delivery instructions configured to cause each of at least a third transducer of the plurality of transducers to deliver a respective second high voltage pulse train of the second high voltage pulse train set with the determined particular parameter set during a second cardiac cycle subsequent to the first cardiac cycle.

In some embodiments, a computer-readable data storage medium system includes one or more computer-readable data storage mediums storing a program executable by one or more data processing devices of a data processing device system of a pulsed field ablation system, the program including first delivery instructions configured to cause, in association with a first state in which a particular cardiac cycle of a patient has a first duration, delivery, via a first pulsed field ablation transducer located on a catheter device, of a first high voltage pulse train during a first particular time interval. A duration of the first particular time interval may be less than the first duration, and the first high voltage pulse train may define a first plurality of high voltage pulses. The first high voltage pulse train may be configured to cause pulsed field ablation of tissue. The program may include second delivery instructions configured to cause, in association with a second state in which the particular cardiac cycle of the patient has a second duration different than the first duration, delivery, via the first pulsed field ablation transducer, of a second high voltage pulse train during a second particular time interval. A duration of the second particular time interval may be less than the second duration. The second high voltage pulse train may define a second plurality of high voltage pulses. The second high voltage pulse train may be configured to cause pulsed field ablation of tissue, and the second plurality of high voltage pulses of the second high voltage pulse train may have a different number of high voltage pulses than the first high voltage pulse train.

In some embodiments, a computer-readable data storage medium system includes one or more computer-readable data storage mediums storing a program executable by one or more data processing devices of a data processing device system of a pulsed field ablation system, the program including identification instructions configured to cause an identification of a first state in which a first plurality of consecutive cardiac cycles of a patient exhibit a non-irregular heart rate. The program may include delivery instructions configured to cause, in association with the first state in which the first plurality of consecutive cardiac cycles of the patient exhibit the non-irregular heart rate, a first pulsed field ablation transducer located on a catheter device to deliver pulsed field ablation energy during each of some, but not all, of the first plurality of consecutive cardiac cycles. The some, but not all, of the first plurality of consecutive cardiac cycles may exclude at least one cardiac cycle of the first plurality of consecutive cardiac cycles during which no pulsed field ablation energy is delivered by the first pulsed field ablation transducer. The excluded at least one cardiac cycle of the first plurality of consecutive cardiac cycles may occur between at least two cardiac cycles of the some, but not all, of the first plurality of consecutive cardiac cycles.

In some embodiments, a computer-readable data storage medium system includes one or more computer-readable data storage mediums storing a program executable by one or more data processing devices of a data processing device system of a pulsed field ablation system, the program including identification instructions configured to cause an identification of a particular pulsed field ablation transducer set of a catheter device. The particular pulsed field ablation transducer set may be identified from a plurality of pulsed field ablation transducers of the catheter device. The particular pulsed field ablation transducer set may be identified to be activated to apply a high voltage pulse train between the pulsed field ablation transducers of the particular pulsed field ablation transducer set. The high voltage pulse train may be sufficient to cause pulsed field ablation of tissue. The program may include first determination and activation instructions configured to cause, in association with a first state in which the identified particular pulsed field ablation transducer set is a first set of pulsed field ablation transducers of the catheter device, determination of a first particular parameter set of the high voltage pulse train and activation of the identified particular pulsed field ablation transducer set to deliver the high voltage pulse train in accordance with the determined first particular parameter set. The program may include second determination and activation instructions configured to cause, in association with a second state in which the identified particular pulsed field ablation transducer set is a second set of pulsed field ablation transducers of the catheter device different than the first set of pulsed field ablation transducers, determination of a second particular parameter set of the high voltage pulse train different than the first particular parameter set and activation of the identified particular pulsed field ablation transducer set to deliver the high voltage pulse train in accordance with the determined second particular parameter set.

In some embodiments, a computer-readable data storage medium system including one or more computer-readable data storage mediums storing a program executable by one or more data processing devices of a data processing device system of a pulsed field ablation system, the program including detection instructions configured to cause detection of a degree of tissue contact exhibited by a portion of a catheter device. The program may include activation instructions configured to cause activation of a particular pulsed field ablation transducer set to deliver a high voltage pulse train. The high voltage pulse train may be sufficient to cause pulsed field ablation of tissue. The program may include first determination instructions configured to cause, in response to a first state in which the detected degree of tissue contact is a first degree, determination of a first particular parameter set of at least the high voltage pulse train. The activation of the particular pulsed field ablation transducer set may be caused to deliver the high voltage pulse train in accordance with the determined first particular parameter set in response to the first state. The program may include determination instructions configured to cause, in response to a second state in which the detected degree of tissue contact is a second degree, determination of a second particular parameter set of at least the high voltage pulse train different than the first particular parameter set. The activation of the particular pulsed field ablation transducer set may be caused to deliver the high voltage pulse train in accordance with the determined second particular parameter set in response to the second state. The first degree may indicate lesser tissue contact than the second degree. The high voltage pulses of the high voltage pulse train delivered in accordance with the first particular parameter set may collectively deliver first energy, and the high voltage pulses of the high voltage pulse train delivered in accordance with the second particular parameter set may collectively deliver second energy. The first energy may be greater than the second energy.

In some embodiments, each of any of one or more of the computer-readable data storage medium systems (also referred to as processor-accessible memory device systems) described herein is a non-transitory computer-readable (or processor-accessible) data storage medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a pulsed field ablation system or a controller system thereof according to various example embodiments, the pulsed field ablation system including a data processing device system, an input-output device system, and a memory device system.

FIG. 2 includes a cutaway diagram of a heart showing a structure of a pulsed field ablation device percutaneously placed in a left atrium of a heart, according to various example embodiments.

FIG. 3A includes a partially schematic representation of a pulsed field ablation system, according to various example embodiments, the pulsed field ablation system representing at least a particular implementation of the pulsed field ablation system of FIG. 1, and the pulsed field ablation system including a structure of a pulsed field ablation device shown in a delivery or unexpanded configuration, according to some embodiments.

FIG. 3B includes a representation of the structure of the pulsed field ablation device of FIG. 3A in a deployed or expanded configuration, according to some embodiments.

FIG. 4 includes a representation of a pulsed field ablation device that includes a flexible circuit structure, according to some embodiments.

FIG. 5A illustrates a simplified portion of an electrocardiogram (ECG) corresponding to at least part of a cardiac cycle.

FIGS. 5B-5D illustrate simplified examples of maintaining average power delivery during a PFA procedure over the course of multiple cardiac cycles, depending on the durations of the cardiac cycles, according to some embodiments.

FIGS. 6A-6G illustrate various methods of performing pulsed field ablation and programmed configurations of a data processing device system of a pulsed field ablation system, according to some embodiments.

FIGS. 7A-7E illustrate various examples of the delivery of high voltage pulses in one or more cardiac cycles by one or more pulsed field ablation systems, according to some embodiments.

FIGS. 8A-8C each respectively illustrate characteristics of a respective portion of a respective high voltage pulse train deliverable by one or more pulsed field ablation systems, according to some embodiments.

FIG. 9 illustrates a comparison between a square waveform and a sinusoidal waveform.

FIG. 10 illustrates an example pulse and a calculation of a rise time thereof, according to some embodiments.

FIG. 11 illustrates a biphasic voltage pulse waveform corresponding to a relatively high load (e.g., tissue) resistance, according to some embodiments.

FIG. 12 illustrates a biphasic voltage pulse waveform corresponding to a relatively lower load (e.g., tissue) resistance, according to some embodiments.

### DETAILED DESCRIPTION

At least some embodiments of the present invention improve upon safety, efficiency, and effectiveness of pulsed field ablation (“PFA”) systems. In some embodiments, undesired thermal effects are at least managed or reduced at least by managing an amount or manner of PFA pulse energy delivery over time. In some embodiments, such managing may include adjusting one or more pulse parameters based on one or more characteristics of one or more cardiac cycles. For example, in some embodiments, it may be desired to deliver PFA pulses within a particular portion or portions of one or more cardiac cycles. In some cases, cardiac cycles may be non-uniform. In this regard, some embodiments of the present invention may adjust one or more PFA pulse parameters based on one or more cardiac cycle characteristics to facilitate maintaining an appropriate level of PFA energy delivery over time that keeps overall procedure time relatively short, while at least reducing undesired thermal effects. It should be noted, however, that the invention is not limited to these, or any other embodiments, or examples provided herein, which are referred to for purposes of illustration only. In this regard, for example, while addressing potential undesired thermal effects may be one benefit of some embodiments of the present invention, such embodiments may have other benefits, and other embodiments may also have at least some of the same or different benefits.

In this regard, in the descriptions herein, certain specific details are set forth in order to provide a thorough understanding of various embodiments of the invention. However, one skilled in the art will understand that the invention may be practiced at a more general level without one or more of these details. In other instances, well known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures or characteristics of different embodiments may be combined in any suitable manner to form one or more other embodiments.

Unless otherwise explicitly noted or required by context, the word “or” is used in this disclosure in a non-exclusive sense. In addition, unless otherwise explicitly noted or required by context, the word “set” is intended to mean one or more. For example, the phrase, “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase, ‘based at least on A’ includes A as well as the possibility of one or more other additional elements besides A. In the same manner, the phrase, ‘based on A’ includes A, as well as the possibility of one or more other additional elements besides A. However, the phrase, ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase, ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers the event A.

The phrase “pulsed field ablation” (“PFA”) as used in this disclosure refers to an ablation method which employs high voltage pulse delivery in a unipolar or bipolar fashion in proximity to target tissue. Each high voltage pulse can be a monophasic pulse including a single polarity, or a biphasic pulse including a first component having a first particular polarity and a second component having a second particular polarity opposite the first polarity. In some embodiments, the second component of the biphasic pulse follows immediately after the first component of the biphasic pulse. In some embodiments, the first and second components of the biphasic pulse are temporally separated by a relatively small time interval. The electric field applied by the high voltage pulses physiologically changes the tissue cells to which the energy is applied (e.g., puncturing or perforating of the cell membrane to form various pores therein). If a lower field strength is established, the formed pores may close in time and cause the cells to maintain viability (e.g., a process sometimes referred to as reversible electroporation). If the field strength that is established is greater, then permanent, and sometimes larger, pores form in the tissue cells, the pores allowing leakage of cell contents, eventually resulting in cell death (e.g., a process sometimes referred to as irreversible electroporation).

The word “fluid” as used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium).

The words “bodily opening” as used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body. The bodily cavity may be a cavity or chamber provided in a bodily organ (e.g., an intracardiac cavity of a heart).

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity. The tissue can include part, or all, of a tissue wall or membrane that defines a surface of the bodily cavity. In this regard, the tissue can form an interior surface of the cavity that surrounds a fluid within the cavity. In the case of cardiac applications, tissue can include tissue used to form an interior surface of an intracardiac cavity such as a left atrium or a right atrium. In some embodiments, the word tissue can refer to a tissue having fluidic properties (e.g., blood) and may be referred to as fluidic tissue.

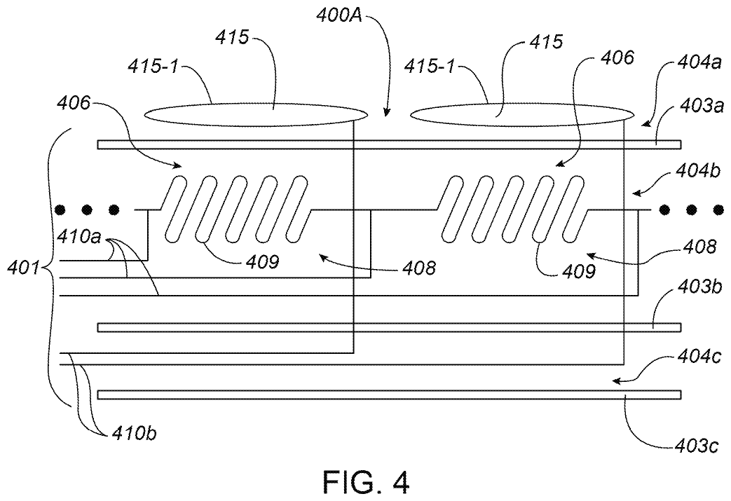

The term “transducer” as used in this disclosure should be interpreted broadly as any device capable of transmitting or delivering energy, distinguishing between fluid and tissue, sensing temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, or any combination thereof. A transducer may convert input energy of one form into output energy of another form. Without limitation, a transducer may include an electrode that functions as, or as part of, a sensing device included in the transducer, an energy delivery device included in the transducer, or both a sensing device and an energy delivery device included in the transducer. A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., with FIG. 4 discussed below.

The term “activation” as used in this disclosure should be interpreted broadly as making active a particular function as related to various transducers disclosed in this disclosure. Particular functions may include, but are not limited to, tissue ablation (e.g., PFA), sensing, sampling or measuring electrophysiological activity (e.g., sensing, sampling or measuring intracardiac electrogram information or sensing, sampling or measuring intracardiac voltage data), sensing, sampling or measuring temperature and sensing, sampling or measuring electrical characteristics (e.g., tissue impedance or tissue conductivity). For example, in some embodiments, activation of a tissue ablation function of a particular transducer is initiated by causing energy sufficient for tissue ablation from an energy source device system to be delivered to the particular transducer. Also, in this example, the activation can last for a duration of time concluding when the ablation function is no longer active, such as when energy sufficient for the tissue ablation is no longer provided to the particular transducer. In some contexts, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

In the following description, some embodiments of the present invention may be implemented at least in part by a data processing device system or a controller system configured by a software program. Such a program may equivalently be implemented as multiple programs, and some, or all, of such software program(s) may be equivalently constructed in hardware. In this regard, reference to “a program” should be interpreted to include one or more programs.

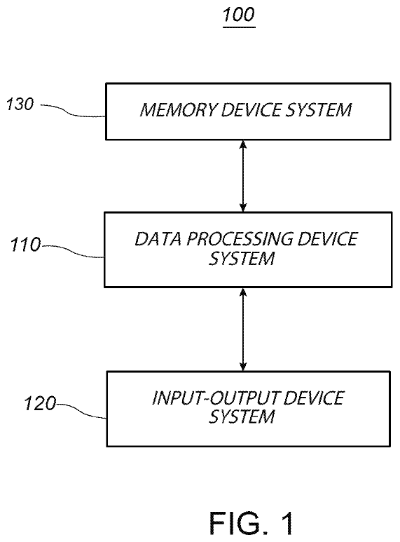

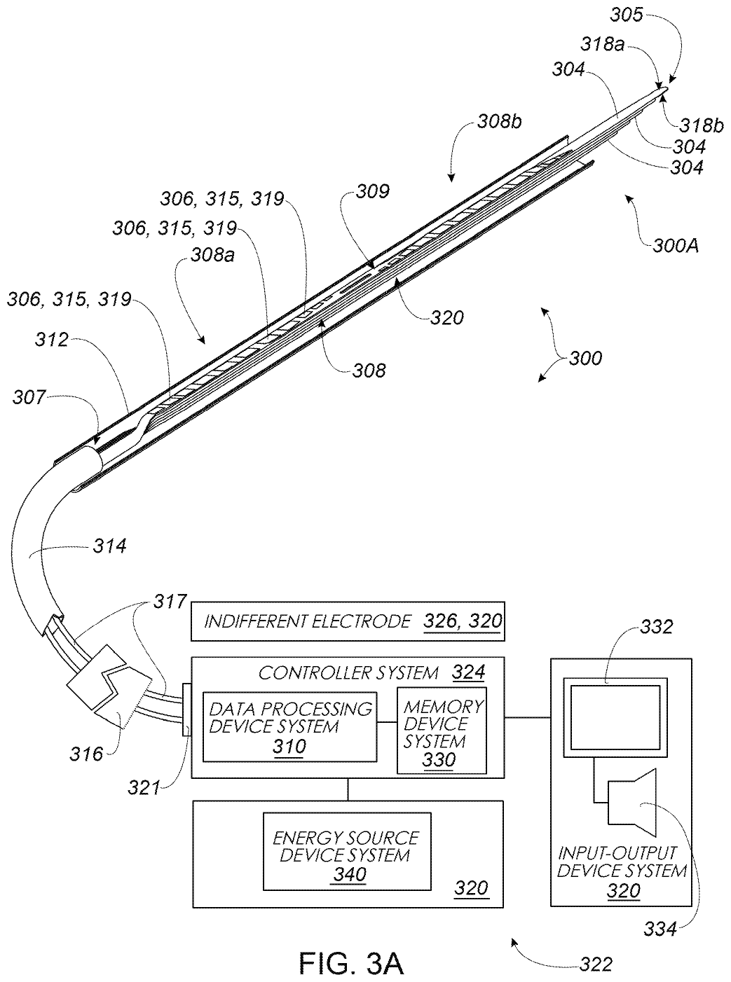

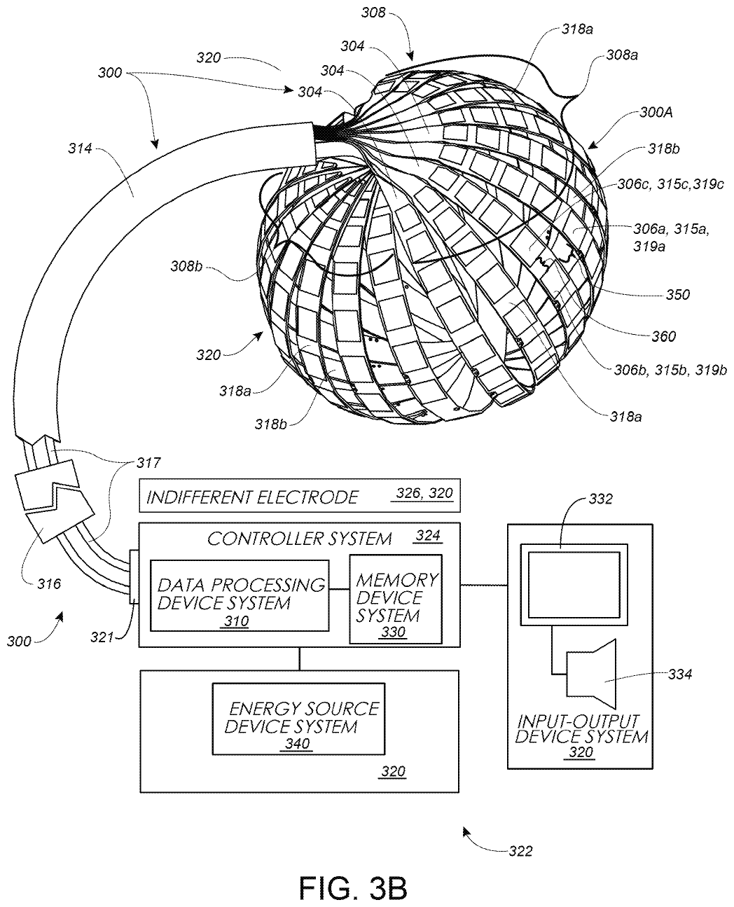

The term “program” in this disclosure should be interpreted as a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130 or 330 shown in FIGS. 1 and 3, respectively. In addition, this disclosure sometimes describes that the instructions or modules of a program are configured to cause the performance of a function. The phrase “configured to” in this context is intended to include at least (a) instructions or modules that are presently in a form executable by one or more data processing devices to cause performance of the function (e.g., in the case where the instructions or modules are in a compiled and unencrypted form ready for execution), and (b) instructions or modules that are presently in a form not executable by the one or more data processing devices, but could be translated into the form executable by the one or more data processing devices to cause performance of the function (e.g., in the case where the instructions or modules are encrypted in a non-executable manner, but through performance of a decryption process, would be translated into a form ready for execution). The word “module” can be defined as a set of instructions. In some instances, this disclosure describes that the instructions or modules of a program perform a function. Such descriptions should be deemed to be equivalent to describing that the instructions or modules are configured to cause the performance of the function.

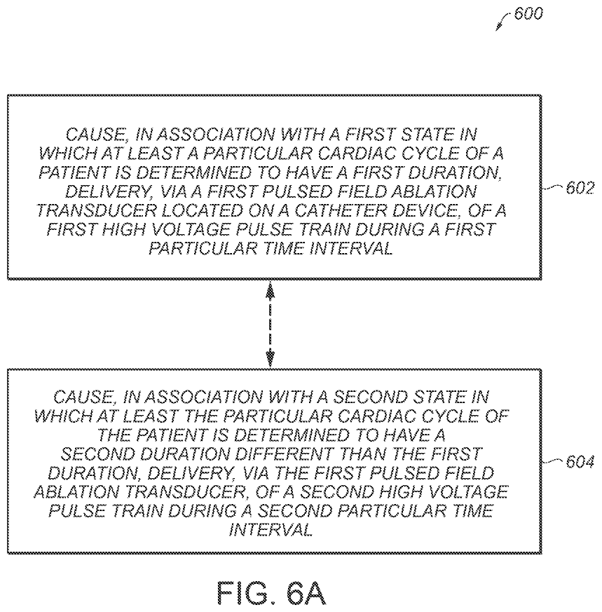

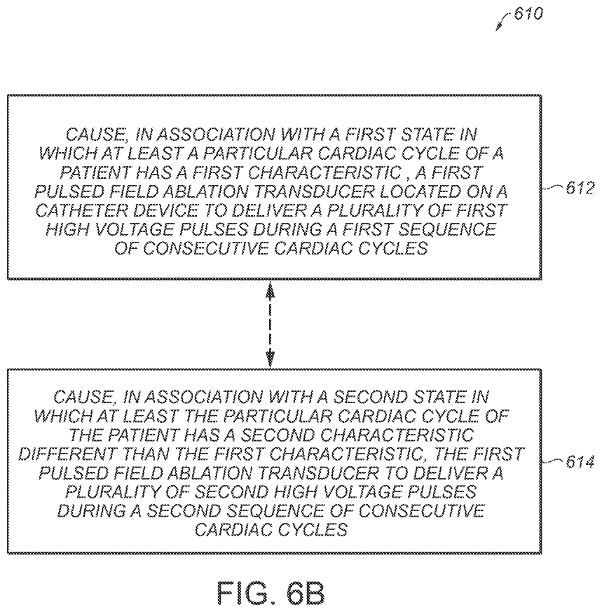

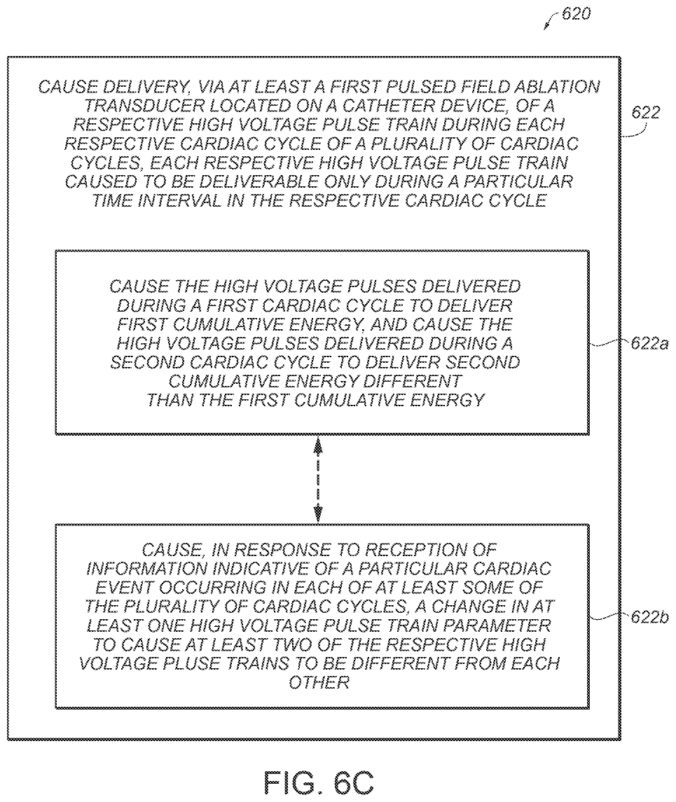

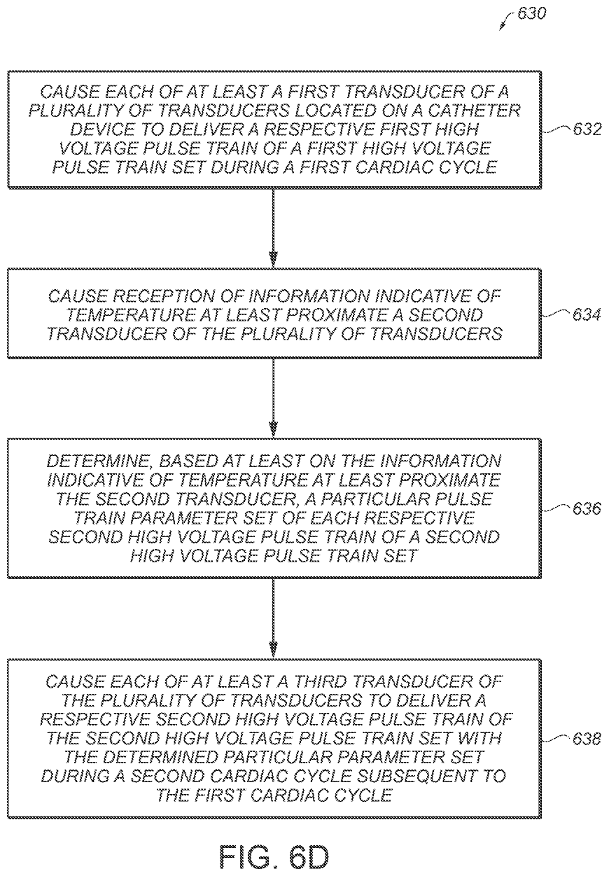

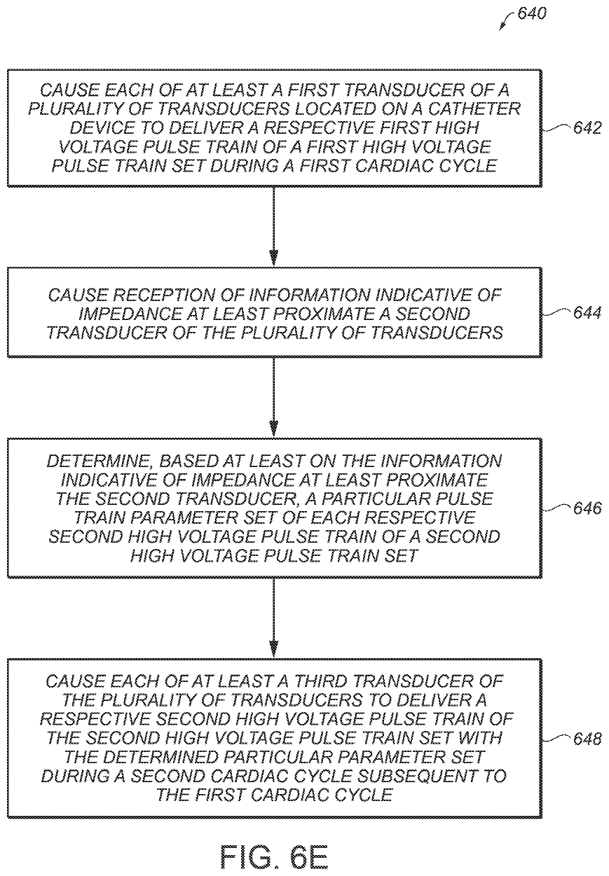

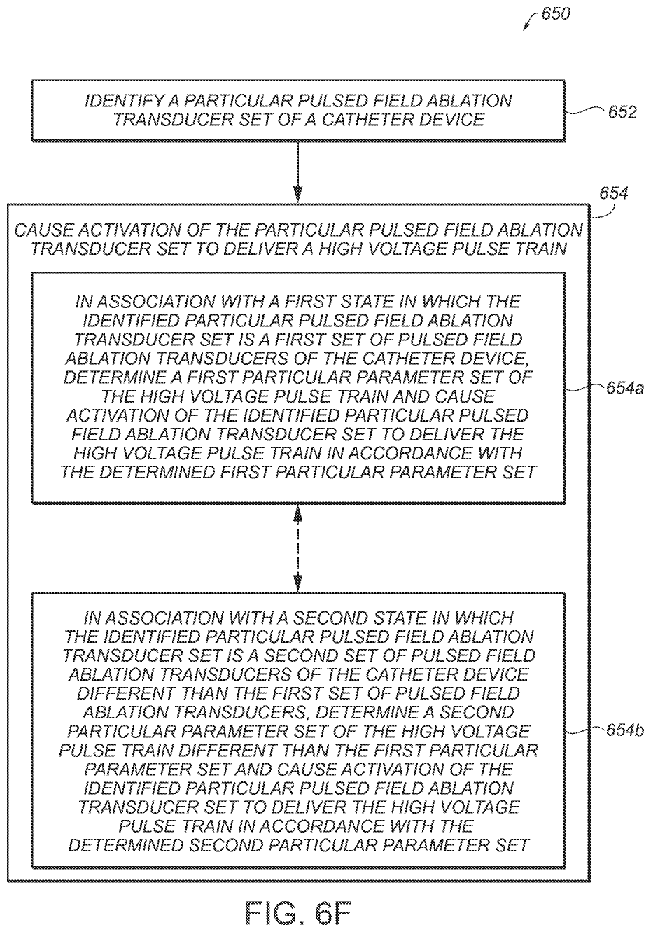

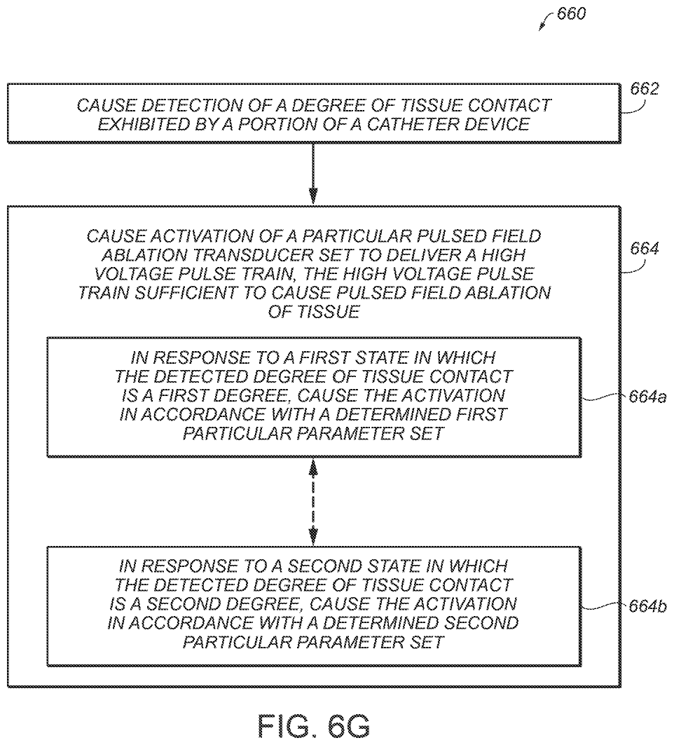

Example methods are described herein with respect to FIGS. 6A-6G. Such figures include blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in each of the method figures herein is not limited to an actual structure of any program or set of instructions or required ordering of method tasks, and such method figures, according to some embodiments, merely illustrate the tasks that instructions are configured to perform, for example, upon execution by a data processing device system in conjunction with interactions with one or more other devices or device systems.

Each of the phrases “derived from” or “derivation of” or “derivation thereof” or the like may be used herein to mean to come from at least some part of a source, be created from at least some part of a source, or be developed as a result of a process in which at least some part of a source forms an input. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof” or the like is used in reference to the information or data. As indicated above, usage of the phrase “derived from” or “derivation of” or “derivation thereof” or the like merely emphasizes the possibility of such changes. Accordingly, in some embodiments, the addition of or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself.

In some embodiments, the term “adjacent”, the term “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the term “adjacent”, the term “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In some embodiments, the term “adjacent”, the term “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the term “adjacent”, the term “proximate”, and the like are not limited to any of the above example definitions, according to some embodiments. In addition, the term “adjacent” and the term “proximate” do not have the same definition, according to some embodiments.

FIG. 1 schematically illustrates a portion of a pulsed field ablation (“PFA”) system or controller system thereof 100 that may be employed to at least select, control, activate, or monitor a function or activation of a number of PFA transducers or electrodes, according to some embodiments. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIGS. 6A-6G. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, or biological components, or otherwise.

The memory device system 130 includes one or more processor-accessible memory devices configured to store one or more programs and information, including the program(s) and information needed to execute the methods or functions described herein, including those described with respect to method FIGS. 6A-6G. The memory device system 130 may be a distributed processor-accessible memory device system including multiple processor-accessible memory devices communicatively connected to the data processing device system 110 via a plurality of computers and/or devices. On the other hand, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” is intended to include or be a non-transitory processor-accessible (or computer-readable) data storage medium. In some embodiments, the processor-accessible memory device system 130 may be considered to include or be a non-transitory processor-accessible (or computer-readable) data storage medium system. And, in some embodiments, the memory device system 130 may be considered to include or be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 can be implemented by a single application-specific integrated circuit (ASIC) in some embodiments.

The input-output device system 120 may include a mouse, a keyboard, a touch screen, another computer, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device system. The phrase “transducer-based device system” is intended to include one or more physical systems that include various transducers. The phrase “transducer-based device” is intended to include one or more physical devices that include various transducers. A PFA device system that includes one or more transducers may be considered a transducer-based device or device system, according to some embodiments.

The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In this regard, the input-output device system 120 may include various other devices or systems described in various embodiments.

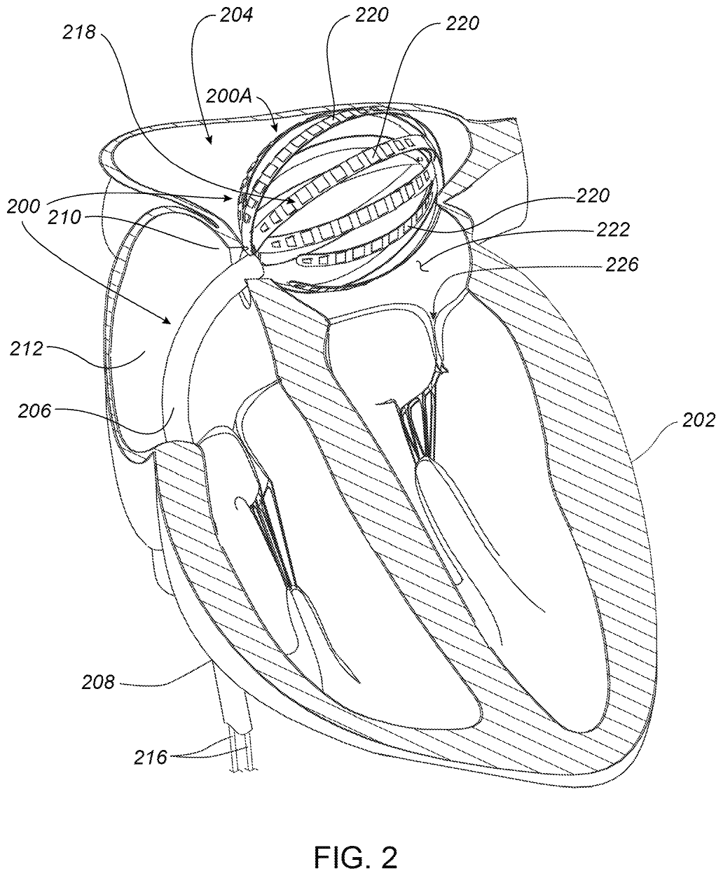

According to some embodiments of the present invention, the system 100 includes some, or all, of the system 200 shown in FIG. 2, or vice versa. In some embodiments, the system 100 includes some, or all, of the system 300 in FIG. 3, or vice versa. In this regard, the system 200, the system 300, or each of the system 200 and the system 300 may be a particular implementation of the system 100, according to some embodiments. Each of at least part of the PFA device system 400A in FIG. 4 may be part of the system 100, the system 200, or the system 300, according to various embodiments.

Various embodiments of transducer-based devices are described herein. Some of the described devices are PFA devices that are percutaneously or intravascularly deployed. Some of the described devices are movable between a delivery or unexpanded configuration (e.g., FIG. 3A discussed below) in which a portion of the device is sized for passage through a bodily opening leading to a bodily cavity, and an expanded or deployed configuration (e.g., FIG. 3B discussed below) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing a therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity.

In some example embodiments, the device includes transducers that sense characteristics (e.g., convective cooling, permittivity, force) that distinguish between fluid, such as a fluidic tissue (e.g., blood), and tissue forming an interior surface of the bodily cavity. Such sensed characteristics can allow a medical system to map the cavity, for example, using positions of openings or ports into and out of the cavity to determine a position or orientation (e.g., pose), or both of the portion of the device in the bodily cavity. In some example embodiments, the described systems employ a navigation system or electro-anatomical mapping system including electromagnetic-based systems and electropotential-based systems to determine a positioning of a portion of a device in a bodily cavity. In some example embodiments, the described devices are capable of ablating tissue in a desired pattern within the bodily cavity using PFA techniques.

In some example embodiments, the devices are capable of sensing various cardiac functions (e.g., electrophysiological activity including intracardiac voltages). In some example embodiments, the devices are capable of providing stimulation (e.g., electrical stimulation) to tissue within the bodily cavity. Electrical stimulation may include pacing.

FIG. 2 is a representation of a PFA system 200 including a PFA device 200A useful in treating a bodily organ, for example, a heart 202, according to one example embodiment.

PFA device 200A can be percutaneously or intravascularly inserted into a portion of the heart 202, such as an intracardiac cavity like left atrium 204. In this example, the PFA device 200A is part of a catheter 206 inserted via the inferior vena cava 208 and penetrating through a bodily opening in transatrial septum 210 from right atrium 212. In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown in some embodiments although more may be present in other embodiments). Electrical conductors 216 provide electrical connections to PFA system 200 that are accessible (e.g., by a controller system or data processing device system) externally from a patient in which the PFA device 200A is inserted.

PFA device 200A includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of PFA system 200 within, or with respect to left atrium 204. For example, transducers 220 may be used to determine a location of pulmonary vein ostia (not shown) or a mitral valve 226, or both. In some embodiments, at least some of the transducers 220 may be used to selectively ablate portions of the tissue 222. In some embodiments, at least some of the transducers 220 may be used to selectively ablate portions of the tissue 222 using PFA. In some embodiments, some of the transducers 220 may be used to ablate a pattern around the bodily openings, ports or pulmonary vein ostia, for instance, to reduce or eliminate the occurrence of atrial fibrillation. In some embodiments, at least some of the transducers 220 are used to ablate cardiac tissue, such as by PFA. In some embodiments, at least some of the transducers 220 are used to sense or sample intracardiac voltage data or sense or sample intracardiac electrogram data. In some embodiments, at least some of the transducers 220 are used to sense or sample intracardiac voltage data or sense or sample intracardiac electrogram data while at least some of the transducers 220 are concurrently ablating cardiac tissue. In some embodiments, at least one of the sensing or sampling transducers 220 is provided by at least one of the ablating transducers 220. In some embodiments, at least a first one of the transducers 220 senses or samples intracardiac voltage data or intracardiac electrogram data at a location at least proximate to a tissue location ablated by at least a second one of the transducers 220. In some embodiments, the first one of the transducers 220 is other than the second one of the transducers 220. In various embodiments, each of at least some of the transducers 220 includes an electrode. In various embodiments, each of at least some of the transducers 220 includes an electrode configured to deliver PFA pulses to tissue.

FIGS. 3A and 3B (collectively, FIG. 3) include a PFA system 300 (e.g., a portion thereof shown schematically) that includes a PFA device 300A, according to one illustrated embodiment. Each of FIGS. 3A and 3B may represent one or more implementations of the medical device system 100 of FIG. 1, according to some embodiments. In this regard, the PFA system 300 in each of FIGS. 3A and 3B may be configured to deliver energy to each of one or more elements, such as one or more transducers or one or more electrodes. The PFA device 300A may include at least a hundred electrodes 315, but need not include that many or may include more. FIG. 3A illustrates the PFA device 300A in the delivery or unexpanded configuration, according to various example embodiments, and FIG. 3B illustrates the PFA device 300A in the deployed or expanded configuration, according to some embodiments.

In this regard, the PFA device 300A includes a plurality of elongate members 304 (three called out in each of FIGS. 3A and 3B) and a plurality of transducers 306 (three called out in FIG. 3A and three called out in FIG. 3B as 306a, 306b and 306c). In some embodiments, the transducers 306 have the configuration of the transducers 220 in FIG. 2. In some embodiments, the transducers 306 are formed as part of, or are located on, the elongate members 304. In some embodiments, the elongate members 304 are arranged as a frame or structure 308 that is selectively movable between an unexpanded or delivery configuration (e.g., as shown in FIG. 3A) and an expanded or deployed configuration (e.g., as shown in FIG. 3B) that may be used to position elongate members 304 or various one of the transducers 306 against a tissue surface within the bodily cavity or position the elongate members 304 in the vicinity of, or in contact with, the tissue surface.

Although FIGS. 3A and 3B show a particular number of elongate members 304 with respective particular lengths thereof, some embodiments have more or fewer elongate members 304 with the same or different respective particular lengths thereof. In this regard, varying the number and lengths of elongate members 304 can change the overall size of structure 308 and the number and density of transducers 306. For example, in some applications and embodiments, it may be desirable to have a smaller spherical size of structure 308, so that the structure 308 can more readily fit into alcoves of a bodily cavity, and such a smaller spherical size may be achieved by reducing the number of elongate members 304 and/or shortening their respective particular lengths, according to some embodiments.

In some embodiments, the structure 308 has a size in the unexpanded or delivery configuration suitable for percutaneous delivery through a bodily opening (e.g., via catheter sheath 312, shown in FIG. 3A, but not shown in FIG. 3B for purposes of clarity) to the bodily cavity. In some embodiments, structure 308 has a size in the expanded or deployed configuration too large for percutaneous delivery through a bodily opening (e.g., via catheter sheath 312) to the bodily cavity. The elongate members 304 may form part of a flexible circuit structure (e.g., such as a flexible printed circuit board (PCB)). The elongate members 304 may include a plurality of different material layers, and each of the elongate members 304 may include a plurality of different material layers, according various embodiments. The structure 308 may include a shape memory material, for instance, Nitinol. The structure 308 may include a metallic material, for instance, stainless steel, or non-metallic material, for instance polyimide, or both a metallic and a non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose) or both of structure 308 in the bodily cavity, or the requirements for successful PFA of a desired pattern.

The plurality of transducers 306 are positionable within a bodily cavity, for example, by positioning of the structure 308. For instance, in some embodiments, the transducers 306 are able to be positioned in a bodily cavity by movement into, within, or into and within the bodily cavity, with or without a change in a configuration of the plurality of transducers 306 (e.g., a change in a configuration of the structure 308 causes a change in a configuration of the transducers 306 in some embodiments). In some embodiments, the plurality of transducers 306 are arrangeable to form a two- or three-dimensional distribution, grid, or array capable of mapping, ablating, or stimulating an inside surface of a bodily cavity or lumen without requiring mechanical scanning. As shown, for example, in FIG. 3A, the plurality of transducers 306 are arranged in a distribution receivable in a bodily cavity (not shown in FIG. 3A). As shown, for example, in FIG. 3A, the plurality of transducers 306 are arranged in a distribution suitable for delivery to a bodily cavity.

FIG. 4 is a schematic side elevation view of at least a portion of a PFA device system 400A that includes a flexible circuit structure 401 that is employed to provide a plurality of transducers 406 (two called out) according to an example embodiment. Such transducers may correspond to transducers 220 or 306, according to various embodiments. In some embodiments, the flexible circuit structure 401 may form part of a structure (e.g., structure 308) that is selectively movable between a delivery configuration sized for percutaneous delivery and expanded or deployed configurations sized too large for percutaneous delivery. In some embodiments, the flexible circuit structure 401 may be located on, or form at least part of, a structural component (e.g., elongate member 304) of a transducer-based device system.

The flexible circuit structure 401 may be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b and 404c (collectively electrically conductive layers 404) that are interleaved with the flexible layers 403. In some embodiments, each of the electrically conductive layers 404 is patterned to form various electrically conductive elements. For example, in some embodiments, electrically conductive layer 404a is patterned to form a respective electrode 415 of each of the transducers 406. In some embodiments, electrodes 415 correspond to electrodes 315. Electrodes 415 have respective electrode edges 415-1 that form a periphery of an electrically conductive surface associated with the respective electrode 415. It is noted that other electrodes employed in other embodiments may have electrode edges arranged to form different electrode shapes.

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out) having a predetermined electrical resistance. In some embodiments, each resistive member 409 includes a metal having relatively high electrical conductivity characteristics (e.g., copper). In some embodiments, electrically conductive layer 404c is patterned to provide portions of various leads 410b arranged to provide an electrical communication path to electrodes 415. In some embodiments, leads 410b are arranged to pass though vias (not shown) in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver PFA high voltage pulses to various tissue structures within a bodily cavity (not shown in FIG. 4) (e.g., an intracardiac cavity or chamber). The PFA high voltage pulses delivered to the tissue structures may be sufficient for ablating portions of the tissue structures. The PFA high voltage pulses delivered to the tissue may be delivered to cause monopolar pulsed field tissue ablation, bipolar pulsed field tissue ablation, or blended monopolar-bipolar pulsed field tissue ablation by way of non-limiting example.

The energy that is delivered by each high voltage pulse may be dependent upon factors including the electrode location, size, shape, relationship with respect to another electrode (e.g., the distance between adjacent electrodes that deliver the PFA energy), the presence, or lack thereof, of various material between the electrodes, the degree of electrode-to-tissue contact, and other factors. In some cases, a maximum ablation depth resulting from the delivery of high voltage pulses by a relatively smaller electrode is typically shallower than that of a relatively larger electrode.

In some embodiments, each electrode 415 is employed to sense or sample an electrical potential in the tissue proximate the electrode 415 at a same or different time than delivering high voltage output pulses for pulsed field tissue ablation. In some embodiments, each electrode 415 is employed to sense or sample intracardiac voltage data in the tissue proximate the electrode 415. In some embodiments, each electrode 415 is employed to sense or sample data in the tissue proximate the electrode 415 from which an electrogram (e.g., an intracardiac electrogram) may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage in between resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

In some embodiments, each electrode 415 is employed to sense or sample impedance (or an electrical characteristic related to impedance such as voltage or current) of tissue proximate the electrode 415 at a same or different time than delivering high voltage output pulses for pulsed field tissue ablation. For example, in some embodiments, an impedance sensing system including a voltage sensor, a current sensor, or a combination of a voltage and a current sensor may be provided with the electrode 415 electrically connected to the sensor circuit. In various embodiments, a return electrode (e.g., a second electrode 415 or indifferent electrode 326 (described below)) is electronically coupled to the sensor circuit. In various embodiments, a controller system (e.g., 324 described below) is communicatively connected or coupled to or contains the sensor circuit and is configured to cause an electrical signal set to be applied to the electrode 415 electrode and the return electrode, and to receive a signal set from the sensor circuit in response to the applied signal set. According to various embodiments, the controller system 324 is configured to determine impedance based at least on the received signal set. According to various embodiments, the controller system 324 is configured to determine tissue resistance based at least on the received signal set. According to various embodiments, the controller system 324 is configured to determine a tissue dielectric constant based at least on the received signal set.

Referring again to FIGS. 3A and 3B, according to some embodiments, PFA device 300A communicates with, receives power from, or is controlled by a transducer-activation system 322, which may include a controller system 324 and an energy source device system 340. In some embodiments, the controller system 324 includes a data processing device system 310 and a memory device system 330 that stores data and instructions that are executable by the data processing device system 310 to process information received from other components of the PFA system 300 of FIGS. 3A and 3B or to control operation of components of the PFA system 300 of FIGS. 3A and 3B, for example, by activating various selected transducers 306 to perform PFA of tissue or sense tissue characteristics. In this regard, the data processing device system 310 may correspond to at least part of the data processing device system 110 in FIG. 1, according to some embodiments, and the memory device system 330 may correspond to at least part of the memory device system 130 in FIG. 1, according to some embodiments. The energy source device system 340, in some embodiments, is part of an input-output device system 320, which may correspond to at least part of the input-output device system 120 in FIG. 1. The controller system 324 may be implemented by one or more controllers. In some embodiments, the PFA device 300A is considered to be part of the input-output device system 320. The input-output device system 320 may also include a display device system 332, a speaker device system 334, or any other device such as those described above with respect to the input-output device system 120.

In some embodiments, elongate members 304 may form a portion or an extension of control leads 317 that reside, at least in part, in an elongated cable 316 and, at least in part, in a flexible catheter body 314. The control leads terminate at a connector 321 or other interface with the transducer-activation system 322 and provide communication pathways between at least the transducers 306 and the controller 324. The control leads 317 may correspond to electrical conductors 216 in some embodiments.

According to some embodiments, the energy source device system 340 includes a high voltage supply. In this regard, although FIGS. 3A and 3B show a communicative connection between the energy source device system 340 and the controller system 324 (and its data processing device system 310), the energy source device system 340 may also be connected to the transducers 306 via a communicative connection that is independent of the communicative connection with the controller system 324 (and its data processing device system 310). For example, the energy source device system 340 may receive control signals via the communicative connection with the controller system 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through catheter body 314, elongated cable 316 or catheter sheath 312) that does not pass through the controller system 324. In this regard, the energy source device system 340 may provide results of its delivering energy to, receiving energy from, or both delivering energy to and receiving energy from one or more of the transducers 306 to the controller system 324 (and its data processing device system 310) via the communicative connection between the energy source device system 340 and the controller system 324. In some embodiments, some, or all, of the energy source device system 340 may be considered part of the controller system 324.

In any event, the number of energy source devices (e.g., high voltage supplies) in the energy source device system 340 may be fewer than the number of transducers in some embodiments. In some embodiments, the energy source device system 340 may, for example, be connected to various selected transducers 306 to selectively provide energy in the form of electrical current or power, light, or low temperature fluid to the various selected transducers 306 to cause ablation of tissue. The energy source device system 340 may, for example, selectively provide energy in the form of electrical current to various selected transducers 306 and measure a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in various ones of FIG. 3, the indifferent electrode 326 may be communicatively connected to the energy source device system 340 via one or more communication lines in some embodiments. In addition, although shown separately in various ones of FIG. 3, indifferent electrode 326 may be considered part of the energy source device system 340 in some embodiments. In various embodiments, indifferent electrode 326 is positioned on an external surface (e.g., a skin-based surface) of a body that includes the bodily cavity into which at least transducers 306 are to be delivered.

It is understood that input-output device system 320 may include other systems. In some embodiments, input-output device system 320 may optionally include energy source device system 340, PFA device 300A, or both energy source device system 340 and PFA device 300A, by way of non-limiting example. Input-output device system 320 may include the memory device system 330 in some embodiments.

Structure 308 may be delivered and retrieved via a catheter member, for example, a catheter sheath 312. In some embodiments, the structure 308 provides expansion and contraction capabilities for a portion of a medical device (e.g., an arrangement, distribution, or array of transducers 306). The transducers 306 may form part of, be positioned or located on, mounted or otherwise carried on the structure 308 and the structure may be configurable to be appropriately sized to slide within catheter sheath 312 in order to be deployed percutaneously or intravascularly. FIG. 3A shows one embodiment of such a structure, where the elongate members 304, in some embodiments, are stacked in the delivery or unexpanded configuration to facilitate fitting within the flexible catheter sheath 312. In some embodiments, each of the elongate members 304 includes a respective distal end 305 (only one called out in FIG. 3A), a respective proximal end 307 (only one called out in FIG. 3A) and an intermediate portion 309 (only one called out in FIG. 3A) positioned between the proximal end 307 and the distal end 305. Correspondingly, in some embodiments, structure 308 includes a proximal portion 308a and a distal portion 308b. In some embodiments, the proximal and the distal portions 308a, 308b include respective portions of elongate members 304. The respective intermediate portion 309 of each elongate member 304 may include a first or front surface 318a that is positionable to face an interior tissue surface within a bodily cavity and a second or back surface 318b opposite across a thickness of the intermediate portion 309 from the front surface 318a. In some embodiments, each elongate member 304 includes a twisted portion at a location proximate proximal end 307. The transducers 306 may be arranged in various distributions or arrangements in various embodiments. In some embodiments, various ones of the transducers 306 are spaced apart from one another in a spaced apart distribution as shown, for example, in at least FIGS. 3A and 3B. In some embodiments, various regions of space are located between various pairs of the transducers 306. For example, in FIG. 3B the PFA system 300 includes at least a first transducer 306a, a second transducer 306b and a third transducer 306c (all collectively referred to as transducers 306). In some embodiments, each of the first, the second, and the third transducers 306a, 306b and 306c are adjacent transducers in the spaced apart distribution. In some embodiments, the first and the second transducers 306a, 306b are located on different elongate members 304 while the second and the third transducers 306b, 306c are located on a same elongate member 304. In some embodiments, a first region of space 350 is between the first and the second transducers 306a, 306b. In some embodiments, the first region of space 350 is not associated with any physical portion of structure 308. In some embodiments, a second region of space 360 associated with a physical portion of device system 300 (e.g., a portion of an elongate member 304) is between the second and the third transducers 306b, 306c. In some embodiments, each of the first and the second regions of space 350, 360 do not include a transducer or electrode thereof of PFA system 300. In some embodiments, each of the first and the second regions of space 350, 360 do not include any transducer or electrode.

It is noted that other embodiments need not employ a group of elongate members 304 as employed in the illustrated figures. For example, other embodiments may employ a structure including one or more surfaces, at least a portion of the one or more surfaces defining one or more openings in the structure. In these embodiments, a region of space not associated with any physical portion of the structure may extend over at least part of an opening of the one or more openings. In some example embodiments, other structures may be employed to support or carry transducers of a transducer-based device provided by various embodiments described in this disclosure. Basket catheters or balloon catheters may be used to distribute the transducers in a two-dimensional or three-dimensional array. In other example embodiments, other structures may be employed to support or carry transducers of a transducer-based device provided by various flexible circuit structures (e.g., such as the flexible circuit structures associated with FIG. 4, in some embodiments). In some embodiments, an elongated catheter member may be used to distribute the flexible circuit structure-based transducers in a linear or curvilinear array.

In various example embodiments, the energy transmission surface 319 of each electrode 315 is provided by an electrically conductive surface. In some embodiments, each of the electrodes 315 is located on various surfaces of an elongate member 304 (e.g., front surfaces 318a or back surfaces 318b). In some embodiments, various electrodes 315 are located on one, but not both, of the respective front surface 318a and respective back surface 318b of each of various ones of the elongate members 304. For example, various electrodes 315 may be located only on the respective front surfaces 318a of each of the various ones of the elongate members 304. Three of the electrodes 315 are identified as electrodes 315a, 315b, and 315c in FIG. 3B. Three of the energy transmission surfaces 319 are identified as 319a, 319b, and 319c in FIG. 3B. In various embodiments, it is intended or designed to have the entirety of each of various ones of the energy transmission surfaces 319 be available or exposed (e.g., without some obstruction preventing at least some of the ability) to contact non-fluidic tissue at least when structure 308 is positioned in a bodily cavity in the expanded configuration.

In various embodiments, the respective shape of various electrically conductive surfaces (e.g., energy transmission surfaces 319) of various ones of the electrodes 315 vary among the electrodes 315. In various embodiments, one or more dimensions or sizes of various electrically conductive surfaces (e.g., energy transmission surfaces 319) of at least some of the electrodes 315 vary among the electrodes 315. The shape or size variances associated with various ones of the various electrically conductive surfaces of electrodes 315 may be motivated for various reasons. For example, in various embodiments, the shapes or sizes of various ones of the various electrically conductive surfaces of electrodes 315 may be controlled in response to various size or dimensional constraints imposed by structure 308.

It should be noted that the present invention is not limited to any particular PFA device transducer arrangement, and the devices 200A, 300A, 400A are provided for illustration purposes only. Various embodiments may include a delivery of pulsed field ablative energy during different times during a PFA treatment that spans a plurality of cardiac cycles. As employed herein, the phrase “cardiac cycle” refers to a time period of a complete heartbeat from its generation to the beginning of the next beat, and includes the diastole, the systole, and an intervening pause. A frequency of the cardiac cycle is described by the heart rate, which is typically expressed as beats per minute. Diastole represents the period of time when the cardiac muscle is relaxed (e.g., not contracting). During ventricular diastole, blood is passively flowing from the left atrium and right atrium into the left ventricle and right ventricle, respectively. The blood flows through the mitral and tricuspid valves (also known as the atrioventricular valves) separating the atria from the ventricles. The right atrium receives blood from the body through the superior vena cava and inferior vena cava. The left atrium receives oxygenated blood from the lungs through normally four pulmonary veins that enter the left atrium. At the end of atrial diastole, both atria contract, propelling blood into the ventricles. Ventricular systole occurs when the left and right ventricles contract and eject blood into the aorta and pulmonary artery, respectively. During ventricular systole, the aortic and pulmonic valves open to permit ejection into the aorta and pulmonary artery. The atrioventricular valves are closed during ventricular systole, therefore no blood is entering from the ventricles; however, blood continues to enter the atria though the vena cava and pulmonary veins. Throughout the cardiac cycle, atrial blood pressure increases and decreases. The cardiac cycle is coordinated by a series of electrical impulses that are produced by specialized heart cells found within the sinoatrial node.

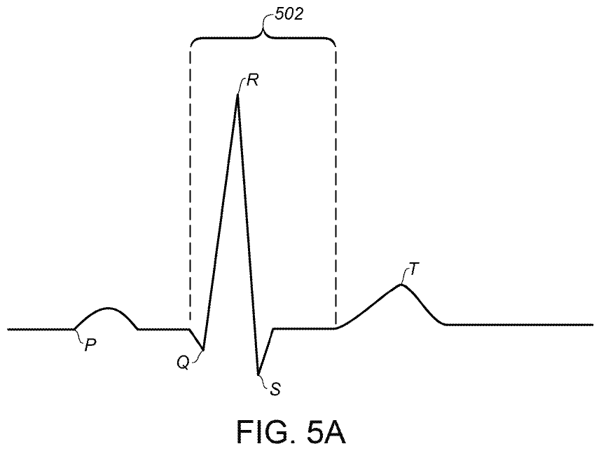

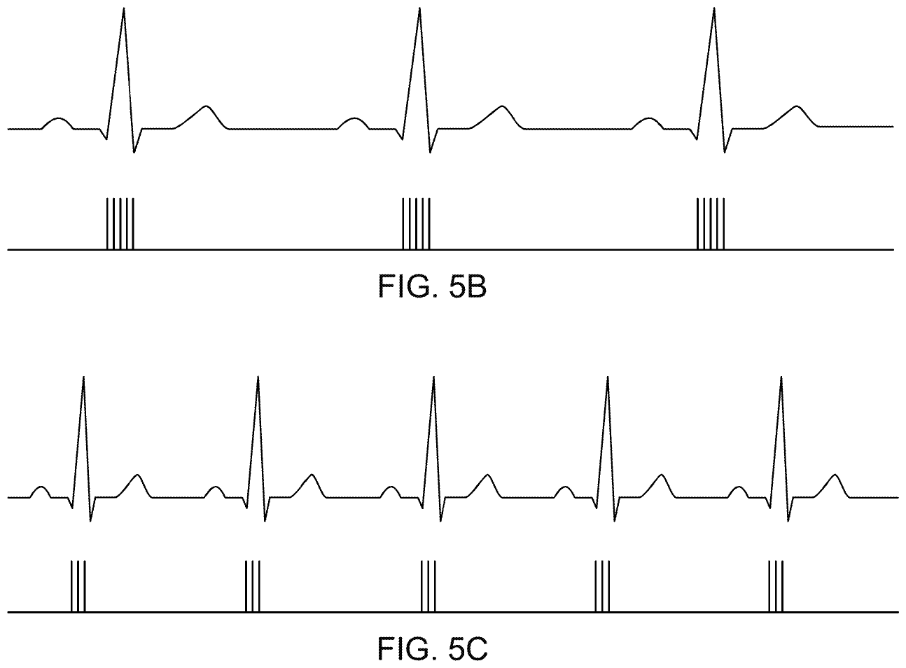

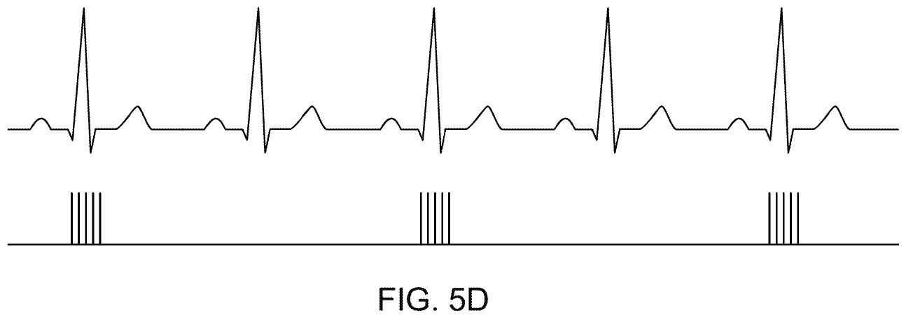

FIG. 5A shows a simplified portion of an electrocardiogram (ECG) corresponding to at least part of a cardiac cycle. The electrocardiogram (ECG) graphs cardiac voltage versus time and is typically generated by using electrodes externally placed on a patient. Typically, an electrocardiogram (e.g., FIG. 5A) includes five deflections or peaks identified as the P wave, Q wave, R wave, S wave, and T wave, the deflections or peaks collectively forming part of a cardiac cycle. It is noted that a U wave (not shown in FIG. 5A) may follow the T wave in the cardiac cycle, but such U wave is typically of low amplitude and may not be visible in various electrocardiograms. The Q, R, and S waves generally occur in rapid succession, and the combination of three of these waves is typically referred to as the QRS complex. The QRS complex generally corresponds to the depolarization of the right and left ventricles of the heart, and at least the R wave thereof is readily visible in electrocardiograms. The P wave marks a deflection in the electrocardiogram produced by excitation of the atria of the heart, while the T wave represents the repolarization (or recovery) of the ventricles in the electrogram. Ventricular systole begins at the QRS complex, and atrial systole begins at the P wave. Also shown in FIG. 5A is a ventricular refractory period 502 of the cardiac cycle, a period of time when the recently triggered ventricular myocytes are incapable of propagating a second action potential. This is also a period of time during which the ventricles are emptied before the next cardiac cycle. For purposes of clarity, the refractory period 502 shown in FIG. 5A is an absolute ventricular refractory period, although other refractory periods exist, such as an atrial refractory period, and effective and relative refractory periods exist in addition to an absolute refractory period.

As indicated previously, although PFA is considered by some to be a generally non-thermal method for causing cell death, the use of various PFA protocols may cause some degree of thermal damage to tissue of the desired ablation region, absent, e.g., one or more of the control configurations or procedures of one or more embodiments of the present invention. For instance, the present inventors recognized that, when relatively high PFA voltages and/or a relatively large number of pulses are employed to ablate the tissue, clinically significant Joule heating of tissue may be encountered during PFA. In various embodiments, the present inventors recognized that an objective for PFA is the desire to avoid additional thermal effects due to Joule heating. In other words, in some embodiments, there is a desire to avoid causing thermally-induced tissue damage when forming the non-thermal pulsed field lesions desired in typical PFA procedures. However, the present inventors recognized that maximizing or otherwise increasing the efficacy of the PFA procedures typically entails delivering as many pulses as possible or at least increasing the number of pulses up to a safety limit to produce efficacious tissue lesions quickly to reduce procedure time while ensuring patient safety. Accordingly, the present inventors recognized that, in some contexts, an important balance must be struck between procedure effectiveness, pulse delivery time, and safety. The present inventors have determined that this balance may be achieved, in some embodiments and contexts, for example, by delivering an average power that approaches a safety threshold for adverse thermal effects.

Tissue or blood temperature during Joule heating can be driven by the balance between thermal energy delivered by the effect of electrical conduction, and by thermal energy removal by tissue conduction or by convective removal due to flowing blood in major vessels or through capillaries. One may consider a monotonic relationship to exist between the power delivered to the tissue and both the equilibrium temperature achieved and the rate of temperature rise during energy delivery where the tissue is not at a steady-state condition. Consequently, targeting a power delivery rate that is less than the threshold for adverse thermal effects may, in various embodiments and contexts, provide a maximum pulse rate possible under the assumption that potential adverse thermal effects are the limiting factor. According to various embodiments and contexts, a requirement to avoid potential adverse thermal effects may include a predetermined or selected target power level chosen to guide PFA delivery rates in the absence of other considerations (e.g., microbubbles, muscle contractions, etc.).

Where efficacy of a PFA procedure may be found to be a function primarily of the total number of delivered pulses in a particular period of time, it may follow that consistent therapy implies delivering a consistent average pulse rate per unit time. However, patients vary significantly in their heart rate and, therefore, the present inventors recognized that delivery of a constant number of pulses per heartbeat will proportionally vary the power delivered. The present inventors recognized that this variation may induce adverse thermal effects if heart rates are too high (either naturally, or because the subject has a cardiac arrhythmia such as atrial flutter (e.g., where a heart rate of over 200 BPM (beats per minute) may sometimes be observed)). Conversely, the present inventors recognized that a low heart rate may not allow the required threshold number of pulses to achieve effective therapy unless the treatment duration overall is undesirably extended.

According to some embodiments, to compensate for these heart rate effects, the Joule heating power delivered with each heartbeat may be varied depending on the heart rate, such that the average power delivered is constant regardless of heart rate. Varying either the number of pulses per heartbeat or the energy delivered per pulse may be employed to achieve this end, according to some embodiments, as can be seen from the average power calculation of equation (1):

Qₜₒₜ=nₕhᵣEₚ (1)

### Where:

Qₜₒₜ is the total Joule heating power (watts) applied in the surrounding blood and tissue;
nₕ is the number of pulses per heartbeat;
hᵣ is the rate of heartbeats (beats per second); and
Eₚ is the Joules of thermal energy delivered per applied PFA pulse.

The energy per applied PFA pulse may be itself a function of several factors, shown for a square wave pulse in equation (2):

Eₚ=VIt_{d} (2)

Or equivalently in equation (3):

Eₚ=I²Rt_{d} (3)

### Where:

V is the voltage drop across the tissue;
I is the total current passing through the tissue;
R is the integrated resistance of the surrounding tissue; and
t_{d} is the PFA pulse duration.

FIGS. 5B-5D illustrate simplified examples of maintaining average power delivery during a PFA procedure over the course of multiple cardiac cycles, depending on the durations of the cardiac cycles, according to some embodiments of the present invention. Each of FIGS. 5B-5D represents a respective sequence of cardiac cycles and a respective PFA pulse train delivered in the respective cardiac cycle. In this regard, each of FIGS. 5B-5D may be considered to represent a plot of voltage (Y-axis, vertical direction) versus time (X-axis, horizontal direction), where the time scales across FIG. 5B-5D are the same. The voltage scales between the respective cardiac cycle plot and the respective PFA pulse train plot in each of FIGS. 5B-5D are different, e.g., in that the voltage of the PFA pulses are much higher than the voltages expressed in a cardiac cycle. However, for purposes of the simplified examples of FIG. 5B-5D, each PFA pulse is assumed to deliver a same amount of energy as every other pulse shown in FIGS. 5B-5D.

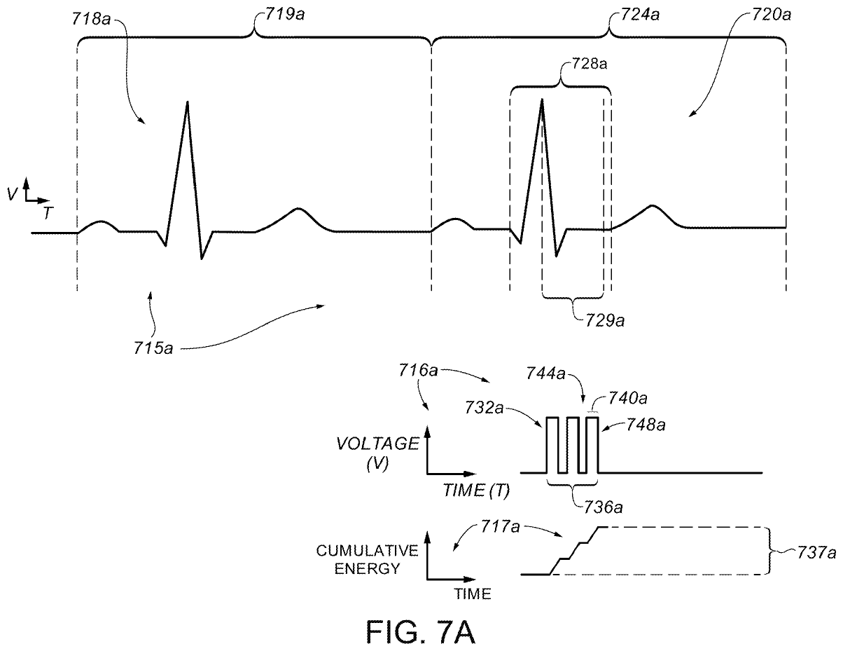

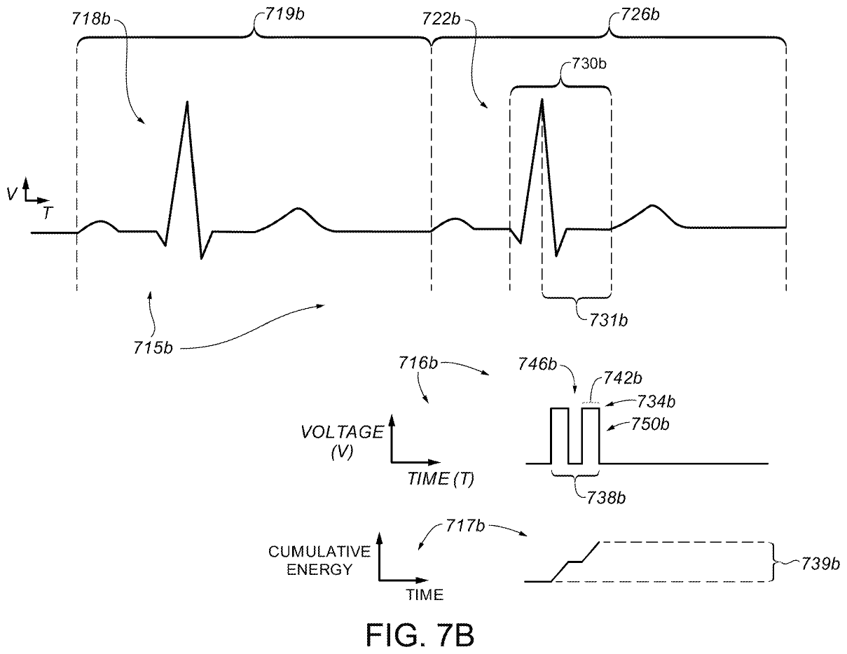

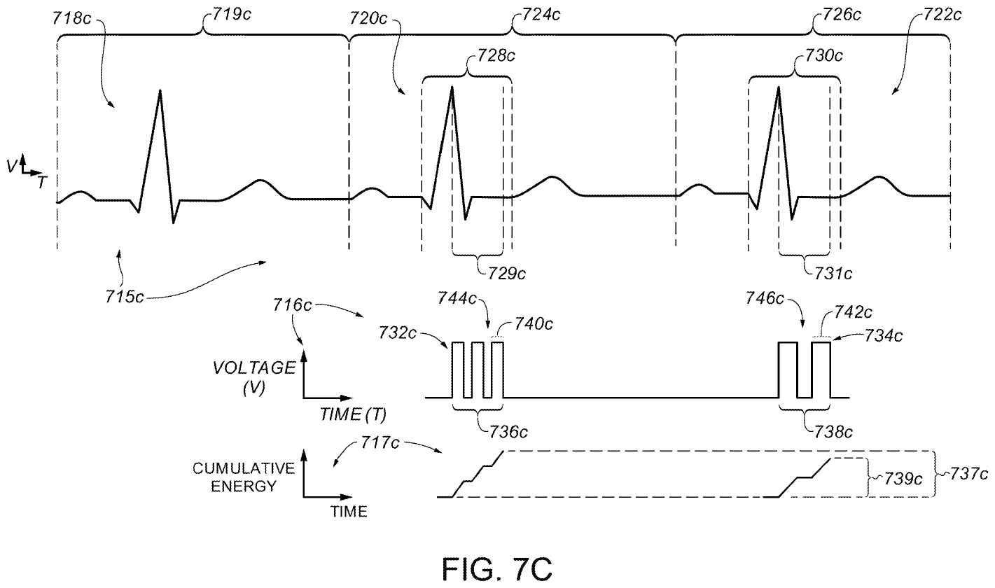

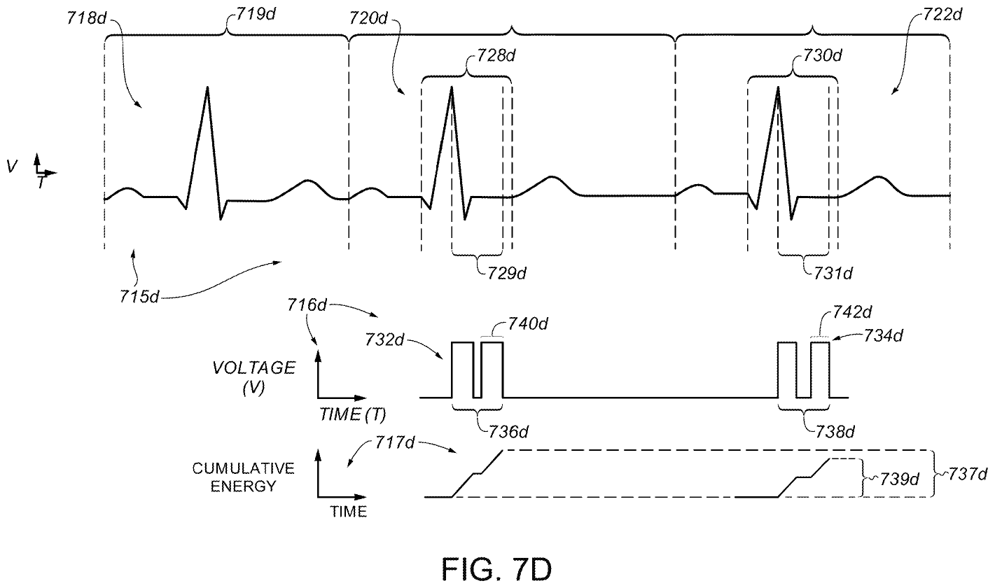

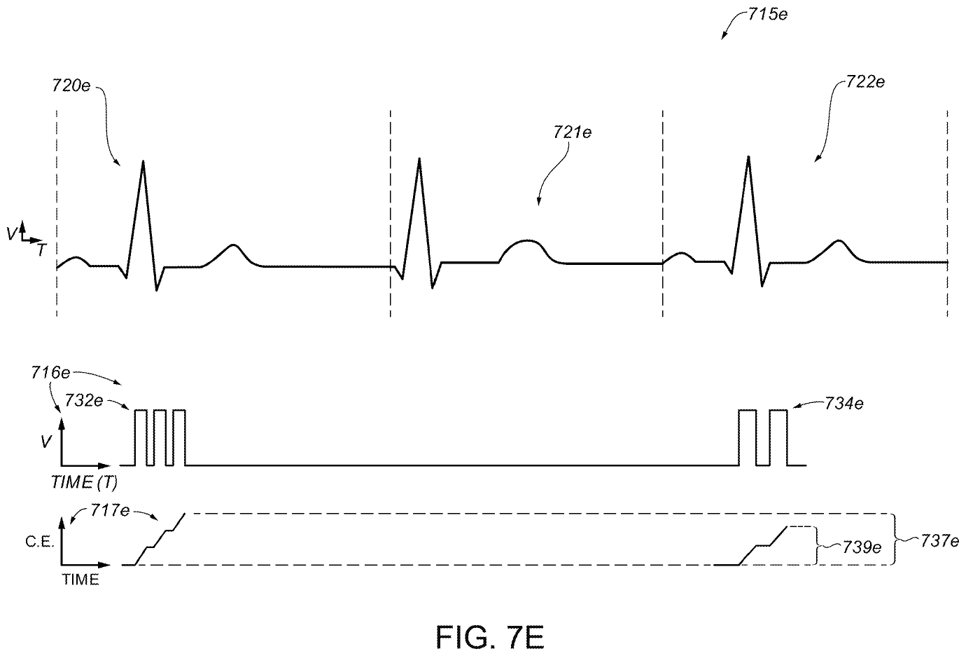

For ease of discussion, each of FIGS. 5B-5D will be assumed to represent one unit of time. FIG. 5B represents an example with three cardiac cycles in one unit of time, FIG. 5C represents an example with five cardiac cycles in the one unit of time, and FIG. 5D represents another example with five cardiac cycles in the one unit of time. In some embodiments, in order to maintain an average PFA energy delivery per unit of time, FIGS. 5A and 5B each illustrate an application of 15 PFA pulses per the unit of time, but because the cardiac cycles in the example of FIG. 5A each have a longer duration than each of the cardiac cycles in the example of FIG. 5B, five PFA pulses are applied as a pulse train in each cardiac cycle in the example of FIG. 5A, whereas three PFA pulses are applied as a pulse train in each cardiac cycle in the example of FIG. 5B. Accordingly, in some embodiments, different numbers of PFA pulses are applied in cardiac cycles, dependent on cardiac cycle duration. In some embodiments, such an approach may be implemented to maintain an average power delivery over multiple cardiac cycles. FIG. 5D illustrates that a PFA pulse train need not be applied in each cardiac cycle, and average power delivery may, nonetheless, be maintained, e.g., by increasing the number of PFA pulses applied in the other cardiac cycles. In the example of FIG. 5D, fifteen PFA pulses are still applied per the same unit of time as with the examples of FIGS. 5B and 5C, even though no PFA pulse is applied in the second and fourth cardiac cycles shown in FIG. 5D, according to some embodiments. While the simplified examples of FIGS. 5B-5D illustrate implementations of identical PFA pulses that merely vary in number, other embodiments vary PFA pulse or pulse train size, shape, or other parameters at least to, in some embodiments, control energy delivery during a PFA procedure. At least some of these features and other features are described in more detail below, with respect to FIGS. 7A-7E, and with reference to FIGS. 6A-6G, according to some embodiments of the present invention. For instance, as discussed further below, FIG. 7A represents an example of a first state in which a particular cardiac cycle 718a has a first duration 719a, and FIG. 7B represents an example of a second state in which a particular cardiac cycle 718b has a second duration 719b, and these different states may result in the delivery of different pulse trains 732a, 734b, respectively, in order to, e.g., maintain a power delivery over multiple cardiac cycles during a PFA procedure, according to some embodiments.

FIG. 6A illustrates a programmed configuration 600 of a data processing device system (e.g., 110, 310), according to some embodiments of the present invention. For example, a programmed configuration may be implemented by the data processing device system being communicatively connected to an input-output device system (e.g., 120, 320) and a memory device system (e.g., 130, 330), and being configured by a program stored by the memory device system at least to perform one or more actions (e.g., such as at least one, more, or all of the actions described in any one of FIG. 6 or otherwise herein). In some embodiments in which the programmed configuration illustrated in FIG. 6A actually is executed at least in part by the data processing device system, such actual execution may be considered a respective method executed by the data processing device system. In this regard, reference numeral 600 and FIG. 6A may be considered to represent one or more methods in some embodiments and, for ease of communication, one or more methods 600 may be referred to at times simply as method 600. The blocks shown in FIG. 6A may be associated with computer-executable instructions of a program that configures the data processing device system to perform the actions described by the respective blocks. According to various embodiments, not all of the actions or blocks shown in FIG. 6A are required, and different orderings of the actions or blocks shown in FIG. 6A may exist. In this regard, in some embodiments, a subset of the blocks shown in FIG. 6A or additional blocks may exist. In some embodiments, a different sequence of various ones of the blocks in FIG. 6A or actions described therein may exist.

In some embodiments, a memory device system (e.g., 130, 330 or a computer-readable medium system) stores the program represented by FIG. 6A, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions described by, or otherwise associated with, the blocks illustrated in FIG. 6A for performance of some or all of method 600 via interaction with at least, for example, a transducer-based device (e.g., PFA devices 200A, 300A, or 400A). In these various embodiments, the program may include instructions configured to perform, or cause to be performed, various ones of the block actions described by or otherwise associated with one or more or all of the blocks illustrated in FIG. 6A for performance of some, or all, of method 600.

FIG. 6A shows configurations of the data processing device system to behave differently in association with different states, respectively referred to by blocks 602, 604. In this regard, either or both of the states and corresponding actions set forth in blocks 602, 604 may actually occur or be executed by the data processing device system (e.g., as in a method) in some embodiments, and, in the case where both states and corresponding actions referred to by blocks 602, 604 actually occur or are executed by the data processing device system, they may occur in any order, as illustrated by the double-headed broken line arrow shown in FIG. 6A between blocks 602, 604, according to various embodiments.

To provide some context in light of FIG. 6A, according to some embodiments, FIG. 7A illustrates a simplified example of (a) a voltage (V) of the electrical activity of a heart versus time (T) plot 715a of an electrocardiogram including a plurality of cardiac cycles 718a and 720a, (b) a voltage versus time plot 716a of voltage pulses, which may be PFA high voltage pulses, and (c) a plot 717a of cumulative energy versus time of the energy delivered by the voltage pulses illustrated in plot 716a. In this regard, the time axes (X-axes) of the plots 715a, 716a, and 717a align in FIG. 7A (and similarly for the corresponding plots in each of FIGS. 7B-7E), but note that the scaling of the voltage or energy axes (Y-axes) of the plots 715a, 716a, 717a may be different (e.g., for ease of illustration). For example, although the heights of the voltage pulses in plot 716a are shown in FIG. 7A (and similarly for the corresponding plots in each of FIGS. 7B-7E) to be shorter than the heights of the R waves of the QRS complexes of the cardiac cycles 718a and 720a, the peak voltages of the voltage pulses in plot 716a in the case of at least some pulsed field ablation (PFA) embodiments are much higher than the peak voltages of the R waves of the QRS complexes of the cardiac cycles 718a and 720a. Further, the electrocardiogram of plot 715a is illustrated as a simplified electrocardiogram for purposes of clarity, and actual electrocardiograms typically have greater variations and noise as compared to that shown in FIG. 7A (and similarly for each of FIGS. 7B-7E). Further, the voltage pulses of plot 716a in FIG. 7A (and similarly for the corresponding plots in each of FIGS. 7B-7E) also are illustrated in a simplified manner for illustration purposes only. For example, voltage pulses of PFA pulse trains typically have a greater, (or in some cases, much greater) frequency (pulses per unit time) and narrower pulse widths than that shown in FIG. 7A (and similarly for each of FIGS. 7B-7E). For another example, the pulses shown in plot 716a in FIG. 7A (and similarly for the corresponding plots in each of FIGS. 7B-7E) have idealized shapes for purposes of clarity and illustration, and actual pulses typically do not have perfect square waves and typically have some variations among pulses. Similarly, the cumulative energy versus time plot 717a in FIG. 7A (and similarly for the corresponding plots in each of FIGS. 7B-7E) also is shown in an idealized manner merely for purposes of clarity and illustration.

In FIG. 6A, according to some embodiments, block 602 represents a configuration of the data processing device system (e.g., data processing device systems 110 or 310) (according to a program) to cause, in association with a first state in which at least a particular cardiac cycle (e.g., particular cardiac cycle 718a in FIG. 7A) of a patient is determined to have a first duration (e.g., duration 719a in FIG. 7A), delivery, (e.g., via the input-output device system (e.g., 120, 320) and via a first pulsed field ablation transducer (e.g., 220, 306, 406) located on a catheter device) of a first high voltage pulse train (e.g., first high voltage pulse train 732a in FIG. 7A) during a first particular time interval (e.g., first particular time interval 729a in FIG. 7A). In some embodiments, a duration of the first particular time interval (e.g., first particular time interval 729a) is less than the first duration (e.g., duration 719a). In some embodiments, the first high voltage pulse train defines a first plurality of high voltage pulses (e.g., represented by a first particular number of high voltage pulses 736a in this example with respect to FIG. 7A). In some embodiments, the first high voltage pulse train is configured to cause pulsed field ablation of tissue. In some embodiments, the first plurality of high voltage pulses is configured to cumulatively deliver first energy (e.g., represented by first energy 737a in FIG. 7A) during the first particular time interval.

In some embodiments, the phrase “pulse train” refers to a series of regular recurrent pulses having the same or similar characteristics. In some embodiments, “regular recurrent pulses” refers to periodic pulses. In some embodiments, “similar characteristics” refers to pulses configured to be the same, but which have relatively slight or minor differences, e.g., due to physical or manufacturing differences in hardware such as electrodes or drivers, or variations in external environment such as characteristics of tissue or blood adjacent the electrodes. In some embodiments, “similar characteristics” refers to pulses configured to cause a same or equivalent effect, such as the production of a lesion in heart tissue that blocks transmission of an electrical signal through the heart tissue for the treatment of atrial fibrillation. In some embodiments, “pulse train” refers to a series of pulses that are closely spaced in time (short inter-pulse time spacing) as compared to the duration of time between the last pulse and the first pulse of separate, adjacent pulse trains (much larger inter-pulse time spacing (e.g., at least five, ten, fifteen, twenty, or more times the average inter-pulse spacing within a pulse train, according to some various embodiments)).

Block 604, according to some embodiments, represents a configuration of the data processing device system (e.g., data processing device systems 110 or 310) to cause, in association with a second state in which at least the particular cardiac cycle of the patient is determined to have a second duration different than the first duration, delivery (e.g., via the input-output device system (e.g., 120, 320) and via the first pulsed field ablation transducer (e.g., 220, 306, 406) of a second high voltage pulse train during a second particular time interval. For instance, while FIG. 7A may represent, according to some embodiments, the above-discussed “first state” in which the particular cardiac cycle is cardiac cycle 718a having first duration 719a, FIG. 7B may represent a second state in which the particular cardiac cycle is cardiac cycle 718b (shown in plot 715b) having a second duration 719b that is different than the first duration 719a of cardiac cycle 718a in FIG. 7A. In association with the second state (e.g., the second state of FIG. 7B in some embodiments), the data processing device system (e.g., data processing device systems 110 or 310) may be configured to cause delivery (e.g., via the input-output device system (e.g., 120, 320) and via the first pulsed field ablation transducer (e.g., 220, 306, 406), of a second high voltage pulse train (e.g., second high voltage pulse train 734b shown in voltage versus time plot 716b) during a second particular time interval (e.g., second particular time interval 731b). In some embodiments, a duration of the second particular time interval (e.g., second particular time interval 731b) is less than the second duration (e.g., second duration 719b). According to some embodiments, the second high voltage pulse train (e.g., second high voltage pulse train 734b) defines a second plurality of high voltage pulses (e.g., represented by a second particular number of high voltage pulses 738b in this example with respect to FIG. 7B). In some embodiments, the second plurality of high voltage pulses of the second high voltage pulse train (e.g., second high voltage pulse train 734b) has a different number (e.g., 738b) of high voltage pulses than the number (e.g., 736a) of high voltage pulses in the first high voltage pulse train (e.g., first high voltage pulse train 732a). According to some embodiments, the second high voltage pulse train is configured to cause pulsed field ablation of tissue. In some embodiments, the second plurality of high voltage pulses is configured to cumulatively deliver second energy (e.g., represented by second energy 739b shown in plot 717b in FIG. 7B) during the second particular time interval (e.g., second particular time interval 731b), the second energy (e.g., second energy 739b) different than the first energy (e.g., first energy 737a shown in FIG. 7A).

In this regard, in some embodiments, depending on the determined duration of a particular cardiac cycle, the data processing device system may be configured to accordingly deliver different amounts of high voltage pulse train energy. For example, if a particular cardiac cycle has, or is expected or predicted to have duration 719a shown in FIG. 7A, the data processing device system (e.g., 110, 310) may be configured to cause delivery of a first high voltage pulse train 732a via an electrode to deliver a first cumulative energy 737a, whereas if the particular cardiac cycle has, or is expected or predicted to have, a different duration 719b shown in FIG. 7B, the data processing device system may be configured to cause the delivery of a second high voltage pulse train 734b via the electrode to deliver a second cumulative energy 739b. Although these examples show a specific correlation between duration 719a and the delivery of first energy 737a, and a specific correlation between duration 719b and the delivery of second energy 739b, other embodiments have different correlations between the duration of the particular cardiac cycle and the pulse train energy delivered.

Such a configuration of the data processing device system to control the amount of pulse train energy delivered based on particular cardiac cycle duration may have various benefits in various contexts and applications including, but not limited to, for example, allowing the data processing device system to maintain a consistent (e.g., average) energy delivery over multiple cardiac cycles, despite duration variations across individual cardiac cycles. Such a configuration may allow delivery of consistent mean energy delivery rate at or near a maximum or desired level up to or considering a safety limit, thereby reducing tissue lesion formation time and reducing overall procedure time, while maintaining a safe procedure.

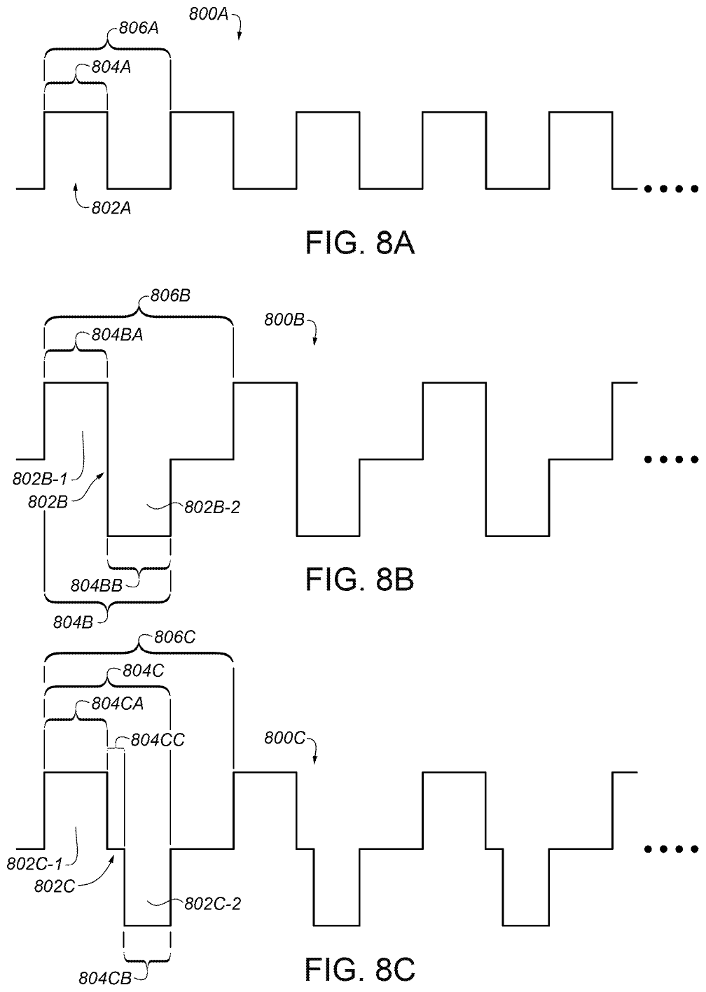

Although the examples of FIG. 7A and FIG. 7B show particular types of pulses in pulse trains 732a, 734b, various pulse train configurations may be employed according to various embodiments. For example, FIG. 8A shows a portion of a pulse train 800A, according to some embodiments. It is noted that the waveforms shown in FIG. 8A, as well as FIG. 8B and FIG. 8C discussed in more detail below, may not be to scale and are merely presented for purposes of illustration. For example, the width (e.g., width 804a) of each pulse shown in FIG. 8A might represent a much smaller fraction of the period (e.g., period 806A) of the respective pulse than that shown in FIG. 8A, in some embodiments. According various embodiments, pulse train 800A includes a plurality of high voltage pulses 802A (only one called out). According to various embodiments, each of the high voltage pulses 802A is a monophasic pulse with each pulse 802A having a same polarity. According to some embodiments, each high voltage pulse 802A has a pulse width 804A. According to various embodiments, the high voltage pulses 802A repeat in pulse train 800A according to period 806A.

FIG. 8B shows a portion of a pulse train 800B according to various embodiments. According to various embodiments, pulse train 800B includes a plurality of high voltage pulses 802B (only one called out). According to some embodiments, each high voltage pulse 802B is a biphasic pulse. For example, in FIG. 8B, each high voltage pulse 802B has a first pulse portion 802B-1 and a second pulse portion 802B-2, the second pulse portion 802B-2 having a different polarity than the polarity of the first pulse portion 802B-1. According to some embodiments, each high voltage pulse 802B has a pulse width 804B. According to various embodiments, pulse width 804B is determined at least by a pulse width 804BA of the first pulse portion 802B-1 and a pulse width 804BB of the second pulse portion 802B-2. According to various embodiments, the high voltage pulses 802B repeat in pulse train 800B according to period 806B.

FIG. 8C shows a portion of a pulse train 800C according to various embodiments. According to some embodiments, pulse train 800C includes a plurality of high voltage pulses 802C (only one called out). According to some embodiments, each high voltage pulse 802C is a biphasic pulse. For example, in FIG. 8C, each high voltage pulse 802C has a first pulse portion 802C-1 and a second pulse portion 802C-2, the second pulse portion 802C-2 having a different polarity than the polarity of the first pulse portion 802C-1. Unlike the biphasic pulses 802B shown in FIG. 8B, the biphasic pulses 802C include an inter-phase gap 804CC between the first pulse portion 802C-1 and the second pulse portion 802C-2. The inter-phase gap 804CC may be motivated for different reasons. For example, in some cardiac ablation procedures a relatively small inter-phase gap 804CC may lead to less muscle twitching while a relatively large inter-phase gap 804CC may be more effective in forming lesions. According to some embodiments, each high voltage pulse 802C has a pulse width 804C. According to various embodiments, pulse width 804C is determined at least by a pulse width 804CA of the first pulse portion 802C-1, a pulse width 804CB of the second pulse portion 802C-2, and the inter-phase gap 804CC. Although FIG. 8C shows different durations for pulse width 804CA and pulse width 804CB, they may instead have the same duration in some embodiments, as is true for the subsequent pulses shown in FIG. 8C. According to various embodiments, the high voltage pulses 802C repeat in pulse train 800C, according to period 806C.

The choice of particular monophasic pulses or biphasic pulses in a particular PFA procedure may be motivated by different reasons, and may vary in different applications. Possible advantages of monophasic pulses may include typically more efficient cellular damage per pulse, (e.g., cell membrane applied charge is not undone with a subsequent phase change), and the possibility of synergy with formed electrolytic products as a pH front forms at each electrode and is driven into the tissue, resulting in deeper lesions or requiring fewer pulses to achieve lesions of a certain depth. Possible advantages of biphasic pulses may include reductions in muscle contractions or nerve stimulation, and less microbubble formation.

In various embodiments, each high voltage pulse in the first high voltage pulse train (e.g., high voltage pulse train 732a or any other pulse train described herein in various embodiments) and each high voltage pulse in the second high voltage pulse train (e.g., high voltage pulse train 734b or any other pulse train described herein in various embodiments) is a high voltage pulse having an amplitude or peak voltage of at least 150 volts. In various embodiments, each high voltage pulse in the first high voltage pulse train and each high voltage pulse in the second high voltage pulse train is a high voltage pulse having an amplitude or peak voltage between 150 volts and 1,000 volts. In various embodiments, each high voltage pulse in the first high voltage pulse train and each high voltage pulse in the second high voltage pulse train is a high voltage pulse having an amplitude or peak voltage between 1,000 volts and 1,500 volts. In various embodiments, each high voltage pulse in the first high voltage pulse train and each high voltage pulse in the second high voltage pulse train is a high voltage pulse having an amplitude or peak voltage between 1,000 volts and 3,000 volts. In various embodiments, the above voltage ranges may apply to each of the positive and negative waveform portions of biphasic PFA high voltage pulses.

In some embodiments (e.g., associated with FIG. 7A), the first high voltage pulse train (e.g., high voltage pulse train 732a) is deliverable during the first particular time interval (e.g., time interval 729a). In some embodiments, the first high voltage pulse train is deliverable entirely within or only during the first particular time interval. In some embodiments, the second high voltage pulse train (e.g., high voltage pulse train 734b) is deliverable during the second particular time interval (e.g., time interval 731b). In some embodiments, the first particular time interval is less than the entirety of the first cardiac cycle. In some embodiments, the second particular time interval is less than the entirety of the second cardiac cycle.

While the examples of FIG. 7A and FIG. 7B show an example of the first particular time interval 729a as being equal to the second particular time interval 731b, other embodiments may have such time intervals be different. For example, in some embodiments, each of the first particular time interval and the second particular time interval has a determined temporal relationship with a particular cardiac event in the particular cardiac cycle. For example, in some embodiments, one possible consideration for delivering a particular PFA pulse train during a particular time interval in a respective cardiac cycle is a desire, in some cases, to apply pulses during a refractory period of the heart. FIG. 7A and FIG. 7B illustrate an example of a refractory period 728a in cardiac cycle 720a and a refractory period 730b in cardiac cycle 722b. In some embodiments, durations of each the first particular time interval and the second particular time interval are predetermined time intervals.

In some embodiments, high voltage pulses delivered outside of the refractory period may risk inducing potentially fatal cardiac arrhythmias due to adverse cardiac stimulation (e.g., ventricular fibrillation). In some cases, cardiac stimulation outside of the refractory period can also cause poorly synchronized heartbeats that can cause deleterious blood pressure drops if delivery of high voltage pulses is prolonged. In some embodiments, high voltage pulses are delivered within a refractory period of the atria. In some embodiments, high voltage pulses are delivered within a refractory period of the ventricles. In some embodiments, high voltage pulses are delivered within a refractory period of both the atria and the ventricles (sometimes referred to as the joint refractory period). Delivery of high voltage pulses during a refractory period, particularly for high voltage pulses that have widely extending electrical fields (e.g., monopolar applications) or which are delivered in close proximity to the heart may be beneficial in some embodiments.

In some embodiments, an initiation of a delivery of a particular pulse train (e.g., the first high voltage pulse train 732a or the second high voltage pulse train 734b) may be gated to a particular cardiac event (e.g., a particular portion of a QRS complex or a P wave (e.g., as by an intracardiac catheter such as a coronary sinus catheter)). For example, in some embodiments, a start of the first particular time interval 729a or the second particular time interval 731b in the particular cardiac cycle or the respective cardiac cycle may be defined in accordance with a pre-determined or determined temporal relationship with a detected particular cardiac event in the particular or respective cardiac cycle. In the example of FIG. 7A, the first particular time interval 729a is shown as gated off of the R wave in the QRS complex of the respective cardiac cycle 720a, and the second particular time interval 731b is shown as gated off of the R wave in the QRS complex of the respective cardiac cycle 722b. However, such gating or temporal relationship need not be based on an event in the same cardiac cycle in which the respective pulse train (e.g., pulse train 732a or pulse train 734b) is delivered, and may instead be gated or have a temporal relationship with a cardiac event in another cardiac cycle, such as the particular cardiac cycle 718a in FIG. 7A or 718b in FIG. 7B in some embodiments.

In some embodiments, the determined temporal relationship with a detected particular cardiac event in the particular or respective cardiac cycle may be configured to cause the first particular time interval or the second particular time interval to occur during a refractory period of the particular cardiac cycle. In some embodiments, the first particular time interval and the second particular time interval have a same temporal relationship with a particular cardiac event in the particular cardiac cycle. In the examples of FIGS. 7A and 7B, the first particular time interval 729a occurs within the refractory period 728a of its respective cardiac cycle 720a, and the second particular time interval 731b occurs within the refractory period 730b of its respective cardiac cycle 722b. Consequently, the application of PFA pulses may be directly affected by the natural or stimulated heart rate of the patient in some embodiments. In at least some embodiments, the particular time intervals (i.e., in which respective PFA pulse trains are applied) are temporally linked to a cardiac event, such particular time intervals may not be the same in duration due to variations in cardiac cycles, in contrast to the equal particular time intervals 729a and 731b illustrated in the examples of FIGS. 7A and 7B. For instance, in some embodiments, the first and second particular time intervals could instead be the entire refractory periods 728a and 730b, respectively, gated to the respective Q wave in the respective QRS complex.

It is noted, in various embodiments, that the first, the second, or each of the first and the second particular time intervals can be gated to the R wave. It is noted, in various embodiments, that the first, the second, or each of the first and the second particular time intervals can be gated to the R wave, such that the respective particular time interval(s) may occur during a refractory period (e.g., a joint atrial and ventricular refractory period). Delivering PFA pulses during a refractory period may protect from (a) atrial arrhythmias, (b) ventricular arrhythmias, or both (a) and (b) occurring during the pulse delivery. Advantageously, gating to the R wave can be accomplished using an external ECG which is present in current procedures, thereby reducing the need for additional devices. It is noted, though, that the refractory period may only provide a relatively short period of time during which a PFA pulse train may be delivered. This in turn may limit the number of pulses that may be delivered during the refractory period, or limit the pulse spacing between adjacent pulses. It is noted that the atrial refractory period is much longer (e.g., approximately 200 ms) than the joint atrial and ventricular refractory period (e.g., approximately 30 ms). Delivering PFA pulses during the atrial refractory period, therefore, allows much more time for pulse delivery within each heartbeat. It is noted, in various embodiments, that the first, the second, or each of the first and the second particular time intervals can be gated to the P wave (also referred to as the atrial wave in some embodiments). It is noted, in various embodiments, that the first, the second, or each of the first and the second particular time intervals can be gated to the P wave, such that the respective particular time interval(s) may occur during a refractory period (e.g., an atrial refractory period). Gating to the P wave can allow more PFA pulses to be delivered during each heartbeat when delivered during the atrial refractory period. This may allow for the completion of the pulse train delivery in fewer heartbeats than if R wave gating was to be employed. Gating to the P wave can allow pulses in each delivered PFA pulse train to be spread further apart (which may lead to reduced microbubble production). It is noted that, while gating to the P wave may protect against atrial arrhythmias, the patient may be susceptible to ventricular arrhythmias during the pulse delivery. In some embodiments, this risk of ventricular arrhythmias may be mitigated to some degree by maintaining sufficient PFA electrode distance from the ventricle, or by limiting delivering the pulse trains over a limited number of heart beats. Although P wave detection may be performed directly from the ECG, the detection of the relatively small signal is harder to automate reliably than the detection of the more easily detectable R wave. According to some embodiments, a reference catheter, such as a coronary sinus catheter or a right atrial reference catheter, may be used to more effectively detect an atrial activation or at least a portion of the P wave. It is noted, in various embodiments, that the first, the second, or each of the first and the second particular time intervals may be gated to an electrical activation signal measured intracardially, such as an electrical activation signal measured by a coronary sinus catheter or by a right atrial reference catheter (by way of non-limiting examples), and that such electrical activation signal may, in some embodiments, represent a fraction of the electrical activation of which the P wave (in the case of atrial activation) or R wave (in the case of ventricular activation) is composed.

Gating to a feature of a cardiac cycle, such as at least the R wave of P wave as discussed above, may be performed at various parts of the respective feature, according to various embodiments. In some embodiments, the gating may be performed at the start, middle, or end of the respective feature, according to some embodiments. For example, P-gating may be performed at the start (such that stimulation by PFA is concurrent with natural activation of the atrium), middle, or at the end of the P-wave (when both atrial chambers are in refractory periods), according to some embodiments.

According to some embodiments, a duration of the first particular time interval (e.g., first particular time interval 729a in some embodiments) may be less than the first duration (e.g., first duration 719a) of the particular cardiac cycle (e.g., cardiac cycle 718a in the “first state” of FIG. 7A). According to some embodiments, a duration of the second particular time interval (e.g., second particular time interval 731b in some embodiments) may be less than the second duration (e.g., second duration 719b) of the particular cardiac cycle (e.g., cardiac cycle 718b in the “second state” of FIG. 7B). In some embodiments, the first particular time interval (e.g., first particular time interval 729a) occurs in a first or respective cardiac cycle (e.g., first cardiac cycle 720a), and the first particular time interval is less than the entirety of the first or respective cardiac cycle (e.g., duration 724a of the first cardiac cycle 720a). In some embodiments, the second particular time interval (e.g., second particular time interval 731b) occurs in a second or respective cardiac cycle (e.g., second particular cardiac cycle 722b), and the second particular time interval is less than the entirety of the second or respective cardiac cycle (e.g., duration 726b of the second particular cardiac cycle 722b). In some embodiments, each of the first particular time interval and the second particular time interval is an uninterrupted time interval, at least as shown, e.g., with respect to first and second particular time intervals 729a and 731b. In some embodiments, a duration of the first particular time interval is defined by a time period required to deliver all the high voltage pulses of the first plurality of high voltage pulses that define the first high voltage pulse train. In some embodiments, a duration of the second particular time interval is defined by a time period required to deliver all the high voltage pulses of the second plurality of high voltage pulses that define the second high voltage pulse train. For example, the first particular time interval 729a is sufficient to include the time for delivery of all pulses in the first high voltage pulse train 732a, and the second particular time interval 731b is sufficient to include the time for delivery of all pulses in the second high voltage pulse train 734b. According to various embodiments, each successive pulse in a pulse train is temporally spaced from an immediately preceding pulse by a same or substantially a same particular inter-pulse time interval (e.g., an inter-pulse time interval equal to the repeating period of the pulses). In some embodiments, a “same or substantially a same inter-pulse time interval” at least in this context is an inter-pulse time interval that does not functionally interfere with the performance of the pulses to collectively act as a PFA pulse train. In some embodiments, the “same or substantially a same inter-pulse time interval” is within 5% of the preceding and next inter-pulse time interval in some embodiments, within 10% of the preceding and next inter-pulse time interval in some embodiments, within 20% of the preceding and next inter-pulse time interval in some embodiments, and, in some embodiments, within 30% of the preceding and next inter-pulse time interval. In some embodiments, if a time interval between pulses exceeds one of these thresholds, whichever is applicable in the respective embodiment or implementation, such an occurrence may be deemed an end or termination of the respective pulse train. In some embodiments, pulse trains themselves are separated by at least the duration of the preceding pulse train in some embodiments, by at least twice the duration of the preceding pulse train in some embodiments, by at least five times the duration of the preceding pulse train in some embodiments, and in some embodiments, by at least ten times the duration of the preceding pulse train. In some embodiments, each pulse train is delivered only during a single heartbeat or single cardiac cycle.

In some embodiments, a duration of the first high voltage pulse train (e.g., first high voltage pulse train 732a) is less than the first duration (e.g., duration 719a in the first state of FIG. 7A in which the particular cardiac cycle is cardiac cycle 718a), the second duration (e.g., duration 719b in the second state of FIG. 7B in which the particular cardiac cycle is cardiac cycle 718b), or each of the first duration and the second duration. For example, the duration of the first high voltage pulse train 732a in FIG. 7A is less than each of the durations 719a and 719b in some embodiments. In some embodiments, a duration of the second high voltage pulse train (e.g., second high voltage pulse train 734b) is less than the first duration, the second duration, or each of the first duration and the second duration. For example, the duration of the second high voltage pulse train 734b in FIG. 7B is less than each of the durations 719a and 719b, in some embodiments.

In some embodiments, the first particular time interval (e.g., first particular time interval 729a) occurs in a particular cardiac cycle (e.g., first cardiac cycle 720a), and no pulse configured to cause PFA is delivered during the particular cardiac cycle outside of the first particular time interval in the particular cardiac cycle. In some embodiments, the first particular time interval (e.g., first particular time interval 729a) occurs in a particular cardiac cycle (e.g., first cardiac cycle 720a), and no pulse configured to cause PFA is delivered by the first pulsed field ablation transducer (e.g., 220, 306, 406) during the particular cardiac cycle outside of the first particular time interval in the particular cardiac cycle. For example, no PFA pulse is delivered outside of the first particular time interval 729a during the first cardiac cycle 720a by the first pulsed field ablation transducer, according to some embodiments. In some embodiments, the second particular time interval (e.g., second particular time interval 731b) occurs in a particular cardiac cycle (e.g., second cardiac cycle 722b), and no pulse configured to cause PFA is delivered during the particular cardiac cycle outside of the second particular time interval in the particular cardiac cycle. In some embodiments, the second particular time interval (e.g., second particular time interval 731b) occurs in a particular cardiac cycle (e.g., second cardiac cycle 722b), and no pulse configured to cause PFA is delivered by the first pulsed field ablation transducer (e.g., 220, 306, 406) during the particular cardiac cycle outside of the second particular time interval in the particular cardiac cycle. For example, no PFA pulse is delivered outside of the second particular time interval 731b during the second particular cardiac cycle 722b by the first pulsed field ablation transducer, according to some embodiments. In some embodiments, the first particular time interval occurs in a particular cardiac cycle, and the first particular time interval has a determined temporal relationship with a particular cardiac event in the particular cardiac cycle. In some embodiments, the second particular time interval occurs in a particular cardiac cycle, and the second particular time interval has a determined temporal relationship with a particular cardiac event in the particular cardiac cycle. For instance, each of the first and second particular time intervals 729a, 731b may have a determined temporal relationship with a particular cardiac event associated with the QRS complex of the respective cardiac cycles 720a, 722b, according to some embodiments.

In some embodiments, the first particular time interval (e.g., time interval 729a) occurs in the first cardiac cycle (e.g., cardiac cycle 720a). In some embodiments, the first particular time interval (e.g., time interval 729a) occurs in a cardiac cycle other than the particular cardiac cycle (e.g., cardiac cycle 720a, which is other than particular cardiac cycle 718a), such that the first high voltage pulse train 732a is delivered in such cardiac cycle other than the particular cardiac cycle, as shown in FIG. 7A. However, in some embodiments, the first particular time interval occurs in the particular cardiac cycle (e.g., particular cardiac cycle 718a), such that the first high voltage pulse train 732a is delivered in or during the particular cardiac cycle. For instance, the duration of the particular cardiac cycle may be estimated in advance or determined partially through the particular cardiac cycle, in order to consequently cause delivery of the appropriate pulse train energy, depending on the duration of the particular cardiac cycle, within the remainder of the particular cardiac cycle. For example, in some embodiments in which the duration of the particular cardiac cycle 718a is estimated, or determined, or predetermined to be duration 719a, as shown by the first state of FIG. 7A, delivery of a high voltage pulse train like pulse train 732a may be caused to be delivered within cardiac cycle 718a, instead of, or in addition to, within cardiac cycle 720a as shown in FIG. 7A.

In some embodiments, the second particular time interval (e.g., the second particular time interval 731b) may occur in a cardiac cycle other than the particular cardiac cycle (e.g., cardiac cycle 722b, which is other than particular cardiac cycle 718b), such that the second high voltage pulse train 734b is delivered in such cardiac cycle (e.g., cardiac cycle 722b) other than the particular cardiac cycle (e.g., particular cardiac cycle 718b in the second state of FIG. 7B). However, in some embodiments, the second particular time interval occurs in the particular cardiac cycle, such that the second high voltage pulse train 734b is delivered in the particular cardiac cycle. For example, in some embodiments in which the duration of the particular cardiac cycle 718b is estimated or predetermined to be duration 719b, as shown by the second state of FIG. 7B, delivery of a high voltage pulse train like pulse train 734b may be caused to be delivered within cardiac cycle 718b, instead of in cardiac cycle 722b as shown in FIG. 7B.

In some embodiments, the first particular time interval is the second particular time interval. For example, in some embodiments, the first particular time interval and the second particular time interval correspond to, or are provided by, a particular time interval in a second cardiac cycle subsequent to the particular cardiac cycle in which a determination of which particular one of the first state (e.g., FIG. 7A in some embodiments) and the second state (e.g., FIG. 7B in some embodiments) exists. For example, in some embodiments, regardless of whether the first state or second state is determined to exist, the first and second particular time intervals may be the same. In some embodiments, the duration of the first particular time interval is the same as the duration of the second particular time interval.

According to some embodiments, the second duration is shorter than the first duration, and the second energy is less than the first energy. For example, the first state of FIG. 7A shows a longer first duration 719a and a greater first energy 737a than the second duration 719b and second energy 739b for the second state of FIG. 7B. In some embodiments, the shorter cardiac cycle duration may correspond to a relatively faster heart rate than a particular heart rate associated with the longer duration. In some embodiments, a lower energy of the high voltage pulse train may be desired to balance out a higher energy from another high voltage pulse train, e.g., to deliver a substantially constant average power regardless of heart rate (for example, as described above or otherwise in this disclosure).

A particular delivery mechanism of the first high voltage pulse train (e.g., first high voltage pulse train 732a) in association with the first state (e.g., the example first state of FIG. 7A in which the particular cardiac cycle is cardiac cycle 718a with duration 719a) may vary among different embodiments. A particular delivery mechanism of the second high voltage pulse train (e.g., second high voltage pulse train 734b) in association with the second state (e.g., the example second state of FIG. 7B in which the particular cardiac cycle is cardiac cycle 718b with duration 719b) may vary among different embodiments. In some embodiments, the data processing device system (e.g., 110, 310) is configured at least by the program at least to cause the delivery, in association with the first state, of the first high voltage pulse train during the particular cardiac cycle, as discussed above. In some embodiments, the data processing device system (e.g., 110, 310) is configured at least by the program at least to cause the delivery, in association with the second state, of the second high voltage pulse train during the particular cardiac cycle, as discussed above. For example, as discussed above, if the particular cardiac cycle is determined to have, or is expected to have either the first duration or the second duration, delivery of the respective one of the first high voltage pulse train and the second high voltage pulse train is made during the particular cardiac cycle, according to some embodiments.

Determination of a duration of the particular cardiac cycle can be performed using various techniques including predictive techniques. In some embodiments, determination of the duration of the particular cardiac cycle is determined based at least on a predicted value of a duration of the particular cardiac cycle. In some embodiments, determination of the duration of a particular cardiac cycle is based on a predictive value determined based at least on a value of a duration of one or more preceding cardiac cycles. In some embodiments, a duration of the particular cardiac cycle may be determined based at least on an average or rolling average of a group of preceding cardiac cycles. In some embodiments, determination of a duration of the particular cardiac cycle may be based at least on a measured value made during the particular cardiac cycle. For example, in some embodiments, a time interval from the last R wave to the P wave of the particular cardiac cycle may be measured, and then a particular additional amount (e.g., an amount of in the order of approximately 150 milliseconds in some embodiments) may be added to the measured time interval to determine the duration of the particular cardiac cycle. In some embodiments, the additional amount is an estimated value. In some embodiments, the additional amount is a measured value derived from previous cardiac cycles of the patient. In some embodiments, the additional amount is a predicted value. In some embodiments, a measured value of a duration of a cardiac cycle may be determined in various ways according to various embodiments. In some embodiments, a duration of a cardiac cycle may be determined from a measured heart rate. Heart rate may be measured by various systems (e.g., an electrocardiogram (ECG), intracardiac reference catheter, or via an intra-arterial pressure sensor).

According to various embodiments, the data processing device system (e.g., 110, 310) is configured at least by the program at least to cause, at least in association with the first state, the first high voltage pulse train (e.g., first high voltage pulse train 732a) to deliver a first average power, and cause, in association with the second state, the second high voltage pulse train (e.g., the second high voltage pulse train 734b) to deliver a second average power. In some embodiment, the second average power is within 5% of the first average power. In some embodiment, the second average power is within 10% of the first average power. In some embodiment, the second average power is within 15% of the first average power. In some embodiment, the second average power is within 20% of the first average power. Keeping the first average power close to the second average power may be motivated by different reasons including, but not limited to, maintaining an overall average PFA power delivery throughout at least a particular portion of the medical procedure being performed, such that the overall average PFA power delivered is at or near a maximum threshold bounded by safety limits. As discussed above, such a configuration may allow for increased lesion formation speed, reduced overall procedure time, and overall increased safety of the patient. In this regard, although in some contexts, increasing overall average PFA power delivery to be at or near the maximum threshold bounded by safety limits is beneficial, such maximization of overall average PFA power delivery may be unnecessary in some other contexts. And yet, even in at least some of these other contexts, maintaining an overall average PFA power delivery may also improve the consistency of lesion formation, even in cases where the overall average PFA power delivery is not set at or near a maximum threshold bounded by safety.

According to some embodiments, one of a first ratio of the first energy to the first duration of the particular cardiac cycle (e.g., a ratio of the first energy 737a to the first duration 719a), and a second ratio of the second energy to the second duration of the particular cardiac cycle (e.g., a ratio of the second energy 739b to the second duration 719b) is within a particular percentage of the other of the first ratio of the first energy to the first duration of the particular cardiac cycle, and the second ratio of the second energy to the second duration of the particular cardiac cycle. In some embodiments in which the duration of the particular cardiac cycle is measured, e.g., instead of predicted per the above discussion, it may be stated that one of a first ratio of the first energy to an actual duration of the particular cardiac cycle and a second ratio of the second energy to the actual duration of the particular cardiac cycle is within a particular percentage of the other of the first ratio of the first energy to the actual duration of the particular cardiac cycle and the second ratio of the second energy to the actual duration of the particular cardiac cycle. In some embodiments, in either the measured or predicted cases, the particular percentage is 5%. In some embodiments, the particular percentage is 10%. In some embodiments, the particular percentage is 15%. In some embodiments, the particular percentage is 20%. Maintaining the closeness of these ratios, according to various embodiments, may have at least the same or similar benefits to maintaining the closeness of the average PFA power delivery discussed above.

In some embodiments, the data processing device system (e.g., 110, 310) is configured at least by the program at least to cause the delivery, in association with the first state (e.g., the example first state of FIG. 7A), of the first high voltage pulse train (e.g., first high voltage pulse train 732a) during a second particular cardiac cycle (e.g., cardiac cycle 720a) subsequent to the particular cardiac cycle (e.g., cardiac cycle 718a). In some embodiments, the data processing device system (e.g., 110, 310) is configured at least by the program at least to cause the delivery, in association with the second state (e.g., the example second state of FIG. 7B), of the second high voltage pulse train (e.g., second high voltage pulse train 734b) during the second particular cardiac cycle subsequent to the particular cardiac cycle (e.g., in the second state of FIG. 7B, the second particular cardiac cycle may be considered cardiac cycle 722b). In some embodiments, (a) a duration of the first high voltage pulse train, (b) a duration of the second high voltage pulse train, or each of (a) and (b) is less than a duration of the second particular cardiac cycle. For instance, in the example first state of FIG. 7A, a duration of the first high voltage pulse train 732a is less than the duration 724a of the second particular cardiac cycle 720a, and in the example of the second state of FIG. 7B, a duration of the second high voltage pulse train 734b is less than the duration 726b of the second particular cardiac cycle, which is cardiac cycle 722b, according to some embodiments.

In some embodiments, the data processing device system (e.g., 110, 310) is configured at least by the program at least to cause, in association with the first state, the first high voltage pulse train to deliver a first average power during the second particular cardiac cycle, and cause, in an association with the second state, the second high voltage pulse train to deliver, during the second particular cardiac cycle, a second average power that maintains the first average power. In some embodiments, the second average power maintains the first average power by being within 5% of the first average power. In some embodiments, the second average power maintains the first average power by being within 10% of the first average power. In some embodiments, the second average power maintains the first average power by being within 15% of the first average power. In some embodiments, the second average power maintains the first average power by being within 20% of the first average power.

In some embodiments, a ratio of the first energy (e.g., first energy 737a) to a duration (e.g., duration 724a) of the second particular cycle (e.g., cardiac cycle 720a in the example first state of FIG. 7A) is a first ratio. In some embodiments, a ratio of the second energy (e.g., second energy 739b) to the duration (e.g., duration 726b) of the second particular cardiac cycle (e.g., cardiac cycle 722b in the example second state of FIG. 7B) is a second ratio. According to some embodiments, one of the first ratio of the first energy to the duration of the second particular cardiac cycle, and the second ratio of the second energy to the duration of the second particular cardiac cycle is within a particular percentage of the other of the first ratio of the first energy to the duration of the second particular cardiac cycle, and the second ratio of the second energy to the duration of the second particular cardiac cycle. In some embodiments, the particular percentage is 5%. In some embodiments, the particular percentage is 10%. In some embodiments, the particular percentage is 15%. In some embodiments, the particular percentage is 20%.

In some embodiments, each of the first particular time interval (e.g., first particular time interval 729a in FIG. 7A in some embodiments) and the second particular time interval (e.g., second particular time interval 731b in FIG. 7B in some embodiments) has a determined temporal relationship (for example, as described above or otherwise in this disclosure) with a particular cardiac event in the second particular cardiac cycle (e.g., R peak, P Peak or other electrocardiogram feature in some embodiments of second particular cardiac cycle 720a in the first state of FIG. 7A and the second particular cardiac cycle 722b in the second state of FIG. 7B). It should be noted that although the examples of FIG. 7A and FIG. 7B show their respective second particular cardiac cycles 720a, 722b as being different cardiac cycles, other embodiments may have the second particular cardiac cycles be the same cardiac cycle in each of the first state and the second state. In some embodiments, the first particular time interval and the second particular time interval have a same temporal relationship with a particular cardiac event in the second particular cardiac cycle. In some embodiments, each of the first particular time interval and the second particular time interval is during a refractory period (e.g., refractory period 728a or 730b in some embodiments) in the second particular cardiac cycle.

In some embodiments, the data processing device system (e.g., 110, 310) is configured at least by the program at least to cause, in association with the first state (e.g., the example first state of FIG. 7A), the first high voltage pulse train to have a first particular number (e.g., first particular number 736a) of high voltage pulses. In some embodiments, the data processing device system (e.g., 110, 310) is configured at least by the program at least to cause, in association with the second state (e.g., the example second state of FIG. 7B), the second high voltage pulse train to have a second particular number (e.g., second particular number 738b) of high voltage pulses, the second particular number of high voltage pulses different than the first particular number of high voltage pulses.

Targeting the Joule heating power delivered during PFA can be achieved by several ways according to various embodiments. In some embodiments, the number of high voltage pulses applied per heartbeat are varied, as shown in the simplified examples of FIG. 7A and FIG. 7B, with the first particular number 736a of high voltage pulses showing a different number of pulses than the second particular number 738b of high voltage pulses. For another example, as discussed above with respect to equation (1), a patient with a heart rate of 60 BPM (beats per minute) may receive within each cardiac cycle twice the number of high voltage pulses as a patient with a heart rate of 120 BPM in order to deliver the same average power, according to some embodiments.

In some embodiments, the data processing device system (e.g., 110, 310) is configured at least by the program at least to cause, in association with the first state, the first high voltage pulse train to have a first particular number of high voltage pulses during delivery of the first high voltage pulse train during the first particular time interval (e.g., first particular time interval 729a), and the data processing device system (e.g., 110, 310) is configured at least by the program at least to cause, in association with the second state, the second high voltage pulse train to have a second particular number of high voltage pulses during delivery of the second high voltage pulse train during the second particular time interval (e.g., second particular time interval 731b). Although the examples of FIG. 7A and FIG. 7B show the second particular number 738b of high voltage pulses being less than the first particular number 736a of high voltage pulses, some embodiments of the present invention may have the second particular number of high voltage pulses be greater than the first particular number of high voltage pulses, e.g., depending on, according to some embodiments, various cardiac cycle characteristics, in some embodiments, and depending on overall desired power delivery (e.g., average power delivery in some embodiments). In this regard, the second particular number of high voltage pulses may be different than the first particular number of high voltage pulses, in some embodiments.

While the examples of FIG. 7A and FIG. 7B show that each high voltage pulse in the first high voltage pulse train 732a delivers a respective pulse energy 740a (only one instance of pulse energy 740a shown in FIG. 7A for clarity) that is different than the respective pulse energy 742b delivered by each high voltage pulse in the second high voltage pulse train 734b (only one instance of pulse energy 742b is shown in FIG. 7B for clarity), each high voltage pulse in the first high voltage pulse train may deliver a same pulse energy as each high voltage pulse in the second high voltage train, according to some embodiments. In some embodiments, an inter-pulse spacing (i.e., a spacing between adjacent high voltage pulses, such as inter-pulse spacing 744a) between adjacent high voltage pulses in the first high voltage pulse train (e.g., first high voltage pulse train 732a) is different than an inter-pulse spacing (e.g., inter-pulse spacing 746b) between adjacent high voltage pulses in the second high voltage pulse train (e.g., second high voltage pulse train 734b). Controlling pulse energy (e.g., 740a, 742b), controlling inter-pulse spacing (e.g., 744a, 746b), or controlling both pulse energy and inter-pulse spacing may be tools for controlling an overall average PFA energy delivery, for example, for reasons discussed herein. In some embodiments, maximally or otherwise increasing spacing between the high voltage pulses (e.g., within the joint atrial/ventricular refractory period) may be employed to minimize or otherwise reduce microbubble formation and transient heating.

Another manner in which Joule heating power may be targeted, according to some embodiments, is by varying an amount of pulse energy delivered by each of at least some of the high voltage pulses. Per equation (2) above, it is also possible to vary the voltage and pulse duration to achieve the same target power as per some embodiments. Combinations of adjustments made to various pulse train parameters such as the voltage, pulse duration, and pulse rate per heartbeat (or the number of pulses delivered per heartbeat) may also be applied to achieve a desired target power, according to various embodiments. In some embodiments, the data processing device system (e.g., 110, 310) is configured at least by the program at least to cause, in association with the first state, each of at least one high voltage pulse in the first high voltage pulse train to deliver a respective first amount of pulse energy (e.g., first pulse energy 740a). In some embodiments, the data processing device system (e.g., 110, 310) is configured at least by the program at least to cause, in association with the second state, each of at least one high voltage pulse in the second high voltage pulse train to deliver a respective second amount of pulse energy (e.g., second pulse energy 742b), each respective second amount of pulse energy different than the each respective first amount of pulse energy (e.g., such as that shown in the examples of FIG. 7A and FIG. 7B, where the first pulse energy 740a is different than the second pulse energy 742b). In some embodiments, the data processing device system (e.g., 110, 310) is configured at least by the program at least to cause, in association with the first state, each high voltage pulse in the first high voltage pulse train to deliver a respective first amount of pulse energy (e.g., first pulse energy 740a). In some embodiments, the data processing device system (e.g., 110, 310) is configured at least by the program at least to cause, in association with the second state, each high voltage pulse in the second high voltage pulse train to deliver a respective second amount of pulse energy (e.g., second pulse energy 742b), each respective second amount of pulse energy different than the each respective first amount of pulse energy. It should be noted that, while FIG. 7A and FIG. 7B merely show the use of brackets about a pulse width to call attention to a pulse energy, e.g., pulse energy 740a and pulse energy 742b merely for simplicity of illustration, it is understood that pulse energy may take into account pulse width (duration), pulse shape (including pulse voltage over time), or both pulse width and pulse shape.

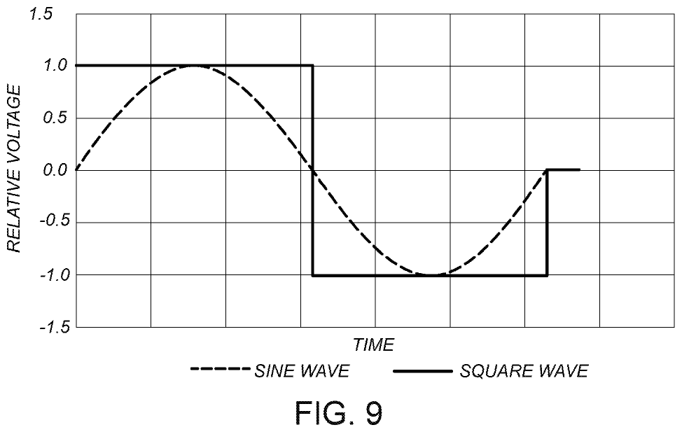

In some embodiments, the high voltage pulse waveform is varied in order to deliver a desired target energy per pulse (e.g., pulse energy) needed to achieve a particular power target. For example, at relatively lower heart rates, a high voltage pulse having a square waveform may be employed, while at relatively higher heart rates, a transition from the square waveform to a sinusoidal waveform may be employed, according to some embodiments. A sinusoidal waveform may allow for delivery of approximately half the energy per pulse for the same maximum voltage even when the average pulse rate is kept constant. FIG. 9 shows a comparison between a square waveform and a sinusoidal waveform.

In some embodiments, the data processing device system (e.g., 110, 310) is configured at least by the program at least to cause, in association with the first state (e.g., the example first state of FIG. 7A), each of at least one high voltage pulse in the first high voltage pulse train (e.g., first high voltage pulse train 732a) to have a respective first pulse shape (e.g., first pulse shape 748a). In some embodiments, the data processing device system (e.g., 110, 310) is configured at least by the program at least to cause, in association with the second state (e.g., the example second state of FIG. 7B), each of at least one high voltage pulse in the second high voltage pulse train (e.g., second high voltage pulse train 734b) to have a respective second pulse shape (e.g., second pulse shape 750b). In some embodiments, each respective second pulse shape is different than each respective first pulse shape. Although the examples of FIG. 7A and FIG. 7B show first high voltage pulse train 732a having a first square wave for each pulse and second high voltage pulse train 734b having a second square wave for each pulse where the first square wave has a different width than the second square wave, various embodiments may vary the shape of each of one or more of each of the first high voltage pulse train 732a and the second high voltage pulse train 734b. For example, various pulse shapes that may be produced include a sinusoidal wave or a square wave, and variations in rise time, fall time, or both rise time and fall time of a high voltage pulse. Such control over pulse shape for one or more pulses may be a tool for controlling energy delivered by each pulse to, e.g., control overall average energy levels applied according to some embodiments. In some embodiments, each respective second pulse shape may be different than each respective first pulse shape in some embodiments, and each respective second pulse shape may be the same as each respective first pulse shape in some embodiments. In some embodiments, the data processing device system (e.g., 110, 310) is configured at least by the program at least to cause, in association with the first state, each high voltage pulse in the first high voltage pulse train to have a respective first pulse shape. In some embodiments, the data processing device system (e.g., 110, 310) is configured at least by the program at least to cause, in association with the second state, each high voltage pulse in the second high voltage pulse train to have a respective second pulse shape, each respective second pulse shape different than each respective first pulse shape.

FIG. 6B illustrates a programmed configuration 610 of a data processing device system (e.g., 110, 310), according to some embodiments of the present invention. In some embodiments in which the programmed configuration illustrated in FIG. 6B actually is executed at least in part by the data processing device system, such actual execution may be considered a respective method executed by the data processing device system. In this regard, reference numeral 610 and FIG. 6B may be considered to represent one or more methods in some embodiments and, for ease of communication, one or more methods 610 may be referred to at times simply as method 610. The blocks shown in FIG. 6B may be associated with computer-executable instructions of a program that configures the data processing device system to perform the actions described by the respective blocks. According to various embodiments, not all of the actions or blocks shown in FIG. 6B are required, and different orderings of the actions or blocks shown in FIG. 6B may exist. In this regard, in some embodiments, a subset of the blocks shown in FIG. 6B or additional blocks may exist. In some embodiments, a different sequence of various ones of the blocks in FIG. 6B or actions described therein may exist.

In some embodiments, a memory device system (e.g., 130, 330 or a computer-readable medium system) stores the program represented by FIG. 6B, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions described by or otherwise associated with the blocks illustrated in FIG. 6B for performance of some or all of method 610 via interaction with at least, for example, a transducer-based device (e.g., PFA devices 200A, 300A, or 400A). In these various embodiments, the program may include instructions configured to perform, or cause to be performed, various ones of the block actions described by or otherwise associated with one or more or all of the blocks illustrated in FIG. 6B for performance of some or all of method 610.

FIG. 6B shows configurations of the data processing device system to behave differently in association with different states, respectively referred to by blocks 612, 614. In this regard, either or both of the states and corresponding actions set forth in blocks 612, 614 may actually occur or be executed by the data processing device system (e.g., as in a method) in some embodiments, and, in the case where both states and corresponding actions referred to by blocks 612, 614 actually occur or are executed by the data processing device system, they may occur in any order, as illustrated by the double-headed broken line arrow shown in FIG. 6B between blocks 612, 614, according to various embodiments.

In FIG. 6B, according to some embodiments, block 612 represents a configuration of the data processing device system (e.g., data processing device system 110 or 310) to cause, in association with a first state in which at least a particular cardiac cycle of a patient has a first characteristic, a first pulsed field ablation transducer (e.g., 220, 306, 406) located on a catheter device to deliver a plurality of first high voltage pulses during a first sequence of consecutive cardiac cycles. In some embodiments, the plurality of first high voltage pulses is configured to deliver a particular average power throughout a duration of the first sequence of consecutive cardiac cycles. For example, with respect to FIG. 7C, the first state may be a state in which a particular cardiac cycle 718c (shown in plot 715c) has a first characteristic, such as a first duration 719c, and the plurality of first high voltage pulses may be the high voltage pulses of both the pulse train 732c and the pulse train 734c across a first sequence of consecutive cardiac cycles 720c, 722c, such that the pulse trains 732c, 734c (shown in plot 716c) deliver a particular average power throughout the duration (duration 724c plus duration 726c) of the first sequence of consecutive cardiac cycles, according to some embodiments.

In FIG. 6B, according to some embodiments, block 614 represents a configuration of the data processing device system (e.g., data processing device system 110 or 310) to cause, in association with a second state in which at least the particular cardiac cycle of the patient has a second characteristic different than the first characteristic, the first pulsed field ablation transducer (e.g., 220, 306, 406) to deliver a plurality of second high voltage pulses during a second sequence of consecutive cardiac cycles. For example, with respect to FIG. 7D, the second state may be a state in which the particular cardiac cycle (e.g., particular cardiac cycle 719d in this state, shown in plot 715d) has a second characteristic, such as a second duration 719d different than first duration 719c, and the plurality of second high voltage pulses in association with this second state may be the high voltage pulses of both pulse train 732d and pulse train 734d (shown in plot 716d) across a second sequence of consecutive cardiac cycles 720d, 722d, such that the pulse trains 732d, 734d deliver a particular average power throughout the duration of the second sequence of cardiac cycles 720d, 722d in the example second state of FIG. 7D. It is noted that each of the first sequence of consecutive cardiac cycles and the second sequence of consecutive cardiac cycles need not be limited to two consecutive cardiac cycles and may include additional cardiac cycles in other embodiments.

According to various embodiments, the delivery of the plurality of second high voltage pulses during the second sequence of consecutive cardiac cycles in association with the second state is configured to maintain the particular average power delivered by the first pulsed field ablation transducer throughout the duration of the first sequence of consecutive cardiac cycles in association with the first state. For purposes of illustration with reference to FIGS. 7C and 7D, cardiac cycle 722c in FIG. 7C and cardiac cycle 718d in FIG. 7D may be considered to be the same cardiac cycle, and the cardiac cycles in FIG. 7D may be considered to occur after (e.g., either immediately after one or more intervening cardiac cycles) the cardiac cycles in FIG. 7C, providing a cumulative sequence of cardiac cycles as follows in this example: a first cardiac cycle 718c, followed by a second cardiac cycle 720c, followed by a third cardiac cycle 722c/718d in some embodiments, followed by a fourth cardiac cycle 720d, followed by a fifth cardiac cycle 722d. In this example, the second high voltage pulse trains 732d, 734d delivered during the second sequence of consecutive cardiac cycles 720d, 722d by the first pulsed field ablation transducer may be configured to maintain the particular average power delivered by the first pulsed field ablation transducer via first high voltage pulse trains 732c, 734c throughout the duration of the first sequence of consecutive cardiac cycles 720c, 722c/718d, in this example, according to some embodiments. According to various embodiments, each of the plurality of first high voltage pulses (e.g., in high voltage pulse trains 732c, 734c) and the plurality of second high voltage pulses (e.g., in high voltage pulse trains 732d, 734d) is configured to cause pulsed field ablation of tissue. According to various embodiments, a first particular ratio of a total number (e.g., represented by number of pulses 736c in high voltage pulse train 732c plus the number of pulses 738c in high voltage pulse train 734c) of the first high voltage pulses to a total number of cardiac cycles in the first sequence of consecutive cardiac cycles (e.g., two cardiac cycles 720c, 722c in the example of FIG. 7C) is different than a second particular ratio of a total number (e.g., represented by number of pulses 736d in high voltage pulse train 732d plus the number of pulses 738d in high voltage pulse train 734d) of the second high voltage pulses to a total number of cardiac cycles in the second sequence of consecutive cardiac cycles (e.g., two cardiac cycles 720d, 722d in the example of FIG. 7D). Differences in these ratios may arise or be produced for different reasons according to various embodiments. According to some embodiments, differences between these ratios may arise or be produced to cause delivery of a target average power throughout at least part of a procedure.

In some embodiments, the total number (e.g., represented by number of pulses 736c in high voltage pulse train 732c plus the number of pulses 738c in high voltage pulse train 734c) of first high voltage pulses in the plurality of first high voltage pulses is different than the total number (e.g., represented by number of pulses 736d in high voltage pulse train 732d plus the number of pulses 738d in high voltage pulse train 734d) of second high voltage pulses in the plurality of second high voltage pulses. However, in other embodiments, the total number of first high voltage pulses in the plurality of first high voltage pulses is the same as the total number of second high voltage pulses in the plurality of second high voltage pulses (e.g., per the example of at least FIG. 5B and FIG. 5C in some embodiments). The total number of high voltage pulses may be different or the same depending on, e.g., an average amount of energy desired to be delivered or the desired manner of control of energy delivered (e.g., number of pulses or pulse shapes), according to various embodiments.

Similarly, in some embodiments, each first high voltage pulse delivers a particular amount of pulse energy that is the same as a particular amount of pulse energy delivered by each second high voltage pulse. However, in other embodiments, each of at least one of the first high voltage pulses delivers a particular amount of pulse energy that is different than a particular amount of pulse energy delivered by each of at least one of the second high voltage pulses. Although the example of FIG. 7C shows different pulse energies 740c, 742c in the plurality of first high voltage pulses, and the example of FIG. 7D shows different pulse energies 740d, 742d in the plurality of second high voltage pulses, some embodiments have each pulse deliver a same amount of pulse energy. In some embodiments, the total number of cardiac cycles in the first sequence of consecutive cardiac cycles is different than the total number of cardiac cycles in the second sequence of consecutive cardiac cycles.

According to some embodiments, the first characteristic indicates at least that the at least the particular cardiac cycle of the patient has a first duration. For example, with respect to FIG. 7C, the first state may be a state in which a particular cardiac cycle 718c has a first characteristic, such as a first duration 719c. According to some embodiments, the second characteristic indicates at least that the at least the particular cardiac cycle of the patient has a second duration different than the first duration. For example, with respect to FIG. 7D, the second state may be a state in which the particular cardiac cycle is cardiac cycle 718d and has a second characteristic, such as a duration 719d different than first duration 719c. In some embodiments, the first characteristic, the second characteristic, or each of the first characteristic and the second characteristic indicates heart rate information. In some embodiments, the first characteristic, the second characteristic, or each of the first characteristic and the second characteristic is determined in accordance with at least measured data, e.g., as discussed above according to some embodiments. In some embodiments, the first characteristic, the second characteristic, or each of the first characteristic and the second characteristic is determined in accordance with at least predictive data, e.g., as discussed above according to some embodiments. In some embodiments, the second duration (e.g., duration 719d) is shorter than the first duration (e.g., duration 719c), and the first particular ratio of the total number of the first high voltage pulses to the total number of cardiac cycles in the first sequence of consecutive cardiac cycles (e.g., cardiac cycles 720c, 722c) is greater than the second particular ratio of the total number of the second high voltage pulses to the total number of cardiac cycles in the second sequence of consecutive cardiac cycles (e.g., cardiac cycles 720d, 722d).

In some embodiments, the plurality of first high voltage pulses includes a plurality of subsets of the first high voltage pulses, each subset of the first high voltage pulses deliverable during a respective cardiac cycle of at least some of the cardiac cycles in the first sequence of consecutive cardiac cycles. For instance, in the example first state shown by FIG. 7C, the plurality of first high voltage pulses may include subsets 732c, 734c of the first high voltage pulses, such that pulse train or subset of pulses 732c is delivered in respective cardiac cycle 720c and pulse train or subset of pulses 734c is delivered in respective cardiac cycle 722c, according to some embodiments. In some embodiments, the plurality of second high voltage pulses includes a plurality of subsets of the second high voltage pulses, each subset of the second high voltage pulses deliverable during a respective cardiac cycle of at least some of the cardiac cycles in the second sequence of consecutive cardiac cycles. For instance, in the example second state shown by FIG. 7D, the plurality of second high voltage pulses may include subsets 732d, 734d of the second high voltage pulses, such that pulse train or subset of pulses 732d is delivered in respective cardiac cycle 720d and pulse train or subset of pulses 734d is delivered in respective cardiac cycle 722d, according to some embodiments. In some embodiments, the first high voltage pulses in each subset of the first high voltage pulses are arranged as a pulse train, such as each of pulse train 732c and pulse train 734c. In some embodiments, the second high voltage pulses in each subset of the second high voltage pulses are arranged as a pulse train, such as each of pulse train 732d and pulse train 734d.

In some embodiments, in association with the first state (e.g., the example first state of FIG. 7C), the data processing device system (e.g., 110, 310) is configured at least by the program at least to cause each respective subset (e.g., each respective subset 732c, 734c) of the plurality of first high voltage pulse trains to be deliverable only during a first particular time interval (e.g., each respective particular time interval 729c, 731c) in the respective cardiac cycle (e.g., each respective cardiac cycle 720c, 722c) of the at least some of the cardiac cycles in the first sequence of consecutive cardiac cycles (e.g., cardiac cycles 720c, 722c), a duration of each first particular time interval shorter than a duration of the respective cardiac cycle of the at least some of the cardiac cycles in the first sequence of consecutive cardiac cycles (e.g., each respective particular time interval 729c, 731c is shorter than the duration of its respective cardiac cycle 720c, 722c in the example of FIG. 7C in some embodiments). In some embodiments, in association with the second state (e.g., the example second state of FIG. 7D), the data processing device system (e.g., 110, 310) is configured at least by the program at least to cause each respective subset (e.g., each respective subset 732d, 734d) of the plurality of second high voltage pulse trains to be deliverable only during a second particular time interval (e.g., each respective particular time interval 729d, 731d) in the respective cardiac cycle (e.g., each respective cardiac cycle 720d, 722d) of the at least some of the cardiac cycles in the second sequence of consecutive cardiac cycles (e.g., cardiac cycles 720d, 722d), a duration of each second particular time interval shorter than a duration of the respective cardiac cycle of the at least some of the cardiac cycles in the second sequence of consecutive cardiac cycles (e.g., each respective particular time interval 729d, 731d is shorter than the duration of its respective cardiac cycle 720d, 722d in the example of FIG. 7D in some embodiments).

In some embodiments, the duration of each first particular time interval is configured to be the same or substantially the same as the duration of each second particular time interval. For instance, in the examples of FIG. 7C and FIG. 7D, each of the first particular time intervals 729c, 731c are the same in duration as each of the second particular time intervals 729d, 731d, according to some embodiments. In this regard, substantially the same may include within 5% according to some embodiments, within 10% according to some embodiments, and within 15% according to some embodiments.

In some embodiments, for each respective cardiac cycle of the at least some of the cardiac cycles in the first sequence of consecutive cardiac cycles, the first particular time interval has a first temporal relationship with a particular cardiac event in the respective cardiac cycle of the at least some of the cardiac cycles in the first sequence of consecutive cardiac cycles. For example, each of the particular time intervals 729c, 731c may have a temporal relationship with a particular cardiac event (e.g., an R wave, P wave or other electrocardiogram feature) in the respective (or other) cardiac cycle, according to various embodiments. In some embodiments, for each respective cardiac cycle of the at least some of the cardiac cycles in the second sequence of consecutive cardiac cycles, the second particular time interval has a second temporal relationship with a particular cardiac event in the respective cardiac cycle of the at least some of the cardiac cycles in the second sequence of consecutive cardiac cycles. For example, each of the particular time intervals 729d, 731d may have a temporal relationship with a particular cardiac event (e.g., an R wave, P wave, or other electrocardiogram feature) in the respective (or other) cardiac cycle, according to various embodiments. In some embodiments, the second temporal relationship is the same as the first temporal relationship. The same, or similar temporal relationships as described above or otherwise herein may be employed according to some embodiments.

In some embodiments, for each respective cardiac cycle of the at least some of the cardiac cycles in the first sequence of consecutive cardiac cycles, the respective first particular time interval (e.g., first particular time interval 729c or first particular time interval 731c) is during a refractory period (e.g., respective refractory period 728c or respective refractory period 730c) in the respective cardiac cycle of the at least some of the cardiac cycles in the first sequence of consecutive cardiac cycles. In some embodiments, for each respective cardiac cycle of the at least some of the cardiac cycles in the second sequence of consecutive cardiac cycles, the respective second particular time interval (e.g., second particular time interval 729d or second particular time interval 731d) is during a refractory period (e.g., respective refractory period 728d or respective refractory period 730d) in the respective cardiac cycle of the at least some of the cardiac cycles in the second sequence of consecutive cardiac cycles.

In some embodiments, the data processing device system (e.g., 110, 310) is configured at least by the program at least to cause, in association with the first state (e.g., the example first state of FIG. 7C), each of at least one first high voltage pulse of the plurality of first high voltage pulses to deliver a respective first amount of pulse energy (e.g., each respective first amount of pulse energy 740c, 742c). In some embodiments, the data processing device system (e.g., 110, 310) is configured at least by the program at least to cause, in association with the second state (e.g., the example second state of FIG. 7D), each of at least one second high voltage pulse of the plurality of second high voltage pulses to deliver a respective second amount of pulse energy (e.g., each respective second amount of pulse energy 740d, 742d). In some embodiments, each respective second amount of pulse energy is the same as each of one, more, or all, of the respective first amounts of pulse energies. In some embodiments, each respective second amount of pulse energy is different than each of one, more, or all, of the respective first amounts of pulse energies. In some embodiments, pulse energies may be configured to be different in a same or similar manner as described above or otherwise herein.

In some embodiments, the data processing device system (e.g., 110, 310) is configured at least by the program at least to cause, in association with the first state, each of at least one first high voltage pulse of the plurality of first high voltage pulses to have a respective first pulse shape. In some embodiments, the data processing device system (e.g., 110, 310) is configured at least by the program at least to cause, in association with the second state, each of at least one second high voltage pulse of the plurality of second high voltage pulses to have a respective second pulse shape. In some embodiments, each respective second pulse shape is different than each respective first pulse shape. The use of different pulse shapes may be motivated for different reasons, including for example in some embodiments, reasons described above or otherwise herein.

The particular delivery of high voltage pulses during each of particular ones of cardiac cycles in a plurality of consecutive cardiac cycles may vary in different embodiments. In this regard, the cardiac cycles of the consecutive cardiac cycles may follow one another in uninterrupted succession or order with no cardiac cycle occurring between successive cardiac cycles of the consecutive cardiac cycles, according to some embodiments. In some embodiments, at least one first high voltage pulse of the plurality of first high voltage pulses is delivered, in accordance with the first state, during each cardiac cycle of the first sequence of consecutive cardiac cycles. For instance, during each cardiac cycle 720c, 722c in the example first state of FIG. 7C, at least one first high voltage pulse (e.g., in pulse train 732c or pulse train 734c, respectively) is delivered. In some embodiments, at least one second high voltage pulse of the plurality of second high voltage pulses is delivered, in accordance with the second state, during each cardiac cycle of the second sequence of consecutive cardiac cycles. For instance, during each cardiac cycle 720d, 722d in the example second state of FIG. 7D, at least one first high voltage pulse (e.g., in pulse train 732d or pulse train 734d, respectively) is delivered.

According to some embodiments, the data processing device system (e.g., 110, 310) is configured at least by the program at least to cause (a) in association with the first state (e.g., the example first state of FIG. 7C), the first pulsed field ablation transducer (e.g., 220, 306, 406) to deliver a respective subset of the plurality of first high voltage pulses during each cardiac cycle of the first sequence of consecutive cardiac cycles, and (b) in association with the second state (e.g., the example second state of FIG. 7D), the first pulsed field ablation transducer (e.g., 220, 306, 406) to deliver a respective subset of the plurality of second high voltage pulses during each cardiac cycle of the second sequence of consecutive cardiac cycles. In some embodiments, the first high voltage pulses in each respective subset of the plurality of first high voltage pulses are arranged in a pulse train. In some embodiments, the second high voltage pulses in each respective subset of the plurality of second high voltage pulses are arranged in a pulse train. In some embodiments, the number of first high voltage pulses in each of at least one of the respective subsets of the plurality of first high voltage pulses is different than the number of second high voltage pulses in each of at least one of the respective subsets of the plurality of second high voltage pulses. For instance, in the examples of FIG. 7C and FIG. 7D, pulse train 732c has a different number of pulses than pulse train 734d in some embodiments. In some embodiments, each of at least one of the respective subsets of the plurality of first high voltage pulses has a first number of the plurality of first high voltage pulses according to various embodiments. In some embodiments, each of at least one of the respective subsets of the plurality of second high voltage pulses has a second number of the plurality of second high voltage pulses, the second number different than the first number according to various embodiments. In some embodiments, the number of first high voltage pulses in each of the respective subsets of the plurality of first high voltage pulses is different than the number of second high voltage pulses in each of the respective subsets of the plurality of second high voltage pulses.

In some embodiments, the first high voltage pulses in each of at least one of the respective subsets of the plurality of first high voltage pulses are configured to cumulatively deliver first energy (e.g., cumulative energy 737c or 739c shown in plot 717c in the example of FIG. 7C) during the respective cardiac cycle of the first sequence of consecutive cardiac cycles, and the second high voltage pulses in each of at least one of the respective subsets of the plurality of second high voltage pulses are configured to cumulatively deliver second energy (e.g., cumulative energy 737d or 739d shown in plot 717d in the example of FIG. 7D) during the respective cardiac cycle of the second sequence of consecutive cardiac cycles. In various embodiments, the second energy is different than the first energy. For example, first energy 737c is different than second energy 739c in the examples of FIG. 7C and FIG. 7D, according to some embodiments.

In some embodiments, a high voltage pulse is not delivered during each heartbeat of a group of consecutive heartbeats or cardiac cycles (e.g., skipping one or more intermediate heartbeats or cardiac cycles in the group of consecutive heartbeats or cardiac cycles). For instance, in the example of FIG. 7E, no high voltage pulse is delivered in cardiac cycle (e.g., an irregular heartbeat in the example of FIG. 7E) 721e in the group of consecutive cardiac cycles 721e, 722e (shown in plot 715e). In some embodiments, a high voltage pulse configured to cause pulsed field ablation of tissue is not delivered during each heartbeat of a group of consecutive heartbeats (e.g., skipping one or more intermediate heartbeats (such as heartbeat 721e) in the group of consecutive heartbeats). This skipping may be a desirable approach at least for cases where the heartbeat is irregular, such as during atrial fibrillation as illustrated in a simplistic manner for purposes of clarity by cardiac cycle 721e in FIG. 7E, such that delivery of desired average power according to some embodiments, is difficult to predict, and as such, may complicate selection of a pulse rate per cardiac cycle. (It is noted in FIG. 7E that, although only a single irregular heartbeat 721e is illustrated between two regular heartbeats 720e, 722e for purposes of clarity of illustration, irregular heartbeats may occur in groups.) This uncertainty may, however, in various embodiments, be controlled over several heartbeats by selectively choosing only a subset of heartbeats to apply high voltage pulses within. Accordingly, in some embodiments, the above-discussed first characteristic of the at least the particular cardiac cycle indicates at least that each of the at least the particular cardiac cycle corresponds to a regular heartbeat and the second characteristic of the particular cardiac cycle indicates at least that each of the at least the particular cardiac cycle corresponds to an irregular heartbeat. For instance, particular cardiac cycle 720e in the example of FIG. 7E corresponds to a regular heartbeat, but in other embodiments where the particular cardiac cycle is something like cardiac cycle 721e, the characteristic of the particular cardiac cycle may indicate that the cardiac cycle is irregular. In this regard, in the case where one or more irregular heartbeats of the patient are detected, increased high voltage pulse energy may then be queued for delivery in one or more subsequent regular heartbeats to account for the lack of delivery of high voltage pulse energy during the one or more irregular heartbeats, so that the overall energy delivery throughout a medical procedure or a portion thereof is maintained at a high level bounded by safety limits in order to reduce procedure time.

It is noted that, in some embodiments, skipping cardiac cycles may also be employed in cases involving regularly repeating heartbeats. For example, in some particular embodiments, it may be considered desirable to control the number of pulses applied per heartbeat to a single value or narrow range (e.g., where microbubble rates are limiting pulses per heartbeat). For example, in some of these particular embodiments, a subject with a regular atrial flutter could have pulses applied only every second or third heartbeat in order to match the average power delivered for a case with a slower, also regular heartbeat (such as a patient in sinus rhythm). By way of non-limiting example, a patient with a heart rate of 60 BPM (beats per minute) may receive within each cardiac cycle of a sequence of consecutive cardiac cycles a particular number of high voltage pulses as compared with a patient with a heart rate of 120 BPM who may receive the same particular number of high voltage pulses within each of every “odd-numbered” cardiac cycle in a sequence of consecutive cardiac cycles (i.e., no high voltage pulse being delivered during any of the “even-numbered” cardiac cycles in the sequence of cardiac cycles, e.g., akin to the state of FIG. 5D). In some embodiments, a same average power is delivered by the high voltage pulses in each case.

In some embodiments, the data processing device system (e.g., 110, 310) may be configured at least by a program at least to cause, in association with a state in which a first plurality of consecutive cardiac cycles of a patient exhibit a non-irregular heart rate, a first pulsed field ablation transducer (e.g., 220, 306, 406) located on a catheter device to deliver pulsed field ablation energy during each of some, but not all, of the first plurality of consecutive cardiac cycles (e.g., as shown by the first, third, and fifth cardiac cycles in FIG. 5D in which pulsed field ablation energy is delivered). In some embodiments, the non-irregular heart rate is a constant heart rate (e.g., as shown in FIG. 5D). In some embodiments, the some, but not all, of the first plurality of consecutive cardiac cycles exclude at least one cardiac cycle of the first plurality of consecutive cardiac cycles during which no pulsed field ablation energy is delivered by the first pulsed field ablation transducer (e.g., as shown by the second and fourth cardiac cycles in FIG. 5D in which no pulsed field ablation energy is delivered). In some embodiments, the excluded at least one cardiac cycle (e.g., the second or fourth cardiac cycles in FIG. 5D) of the first plurality of consecutive cardiac cycles occurs between at least two cardiac cycles of the some, but not all, of the first plurality of consecutive cardiac cycles (e.g., the second cardiac cycle in FIG. 5D in which no pulsed field ablation energy is delivered is between the first and third cardiac cycles in FIG. 5D in which pulsed field ablation energy is delivered).

In some embodiments, the data processing device system (e.g., 110, 310) is configured at least by the program at least to cause, (a) in association with the first state (e.g., akin to FIG. 7E), the first pulsed field ablation transducer (e.g., 220, 306, 406) to deliver a respective subset of the plurality of first high voltage pulses during each cardiac cycle of some but not all of the cardiac cycles of the first sequence of cardiac cycles, the some but not all of the first sequence of consecutive cardiac cycles excluding at least one cardiac cycle (e.g., cardiac cycle 721e) of the first sequence of consecutive cardiac cycles during which no pulsed field ablation energy is delivered by at least the first pulsed field ablation transducer (e.g., 220, 306, 406). In some embodiments, the excluded at least one cardiac cycle (e.g., cardiac cycle 721e) of the first sequence of consecutive cardiac cycles occurs between at least two cardiac cycles of the some, but not all, of the first sequence of consecutive cardiac cycles. In some embodiments, the data processing device system (e.g., 110, 310) is configured at least by the program at least to cause (b) in association with the second state, the first pulsed field ablation transducer (e.g., 220, 306, 406) to deliver a respective subset of the plurality of second high voltage pulses during each cardiac cycle of some, but not all, of the cardiac cycles of the second sequence of consecutive cardiac cycles, the some, but not all, of the second sequence of consecutive cardiac cycles excluding at least one cardiac cycle of the second sequence of consecutive cardiac cycles during which no pulsed field ablation energy is delivered by the first pulsed field ablation transducer. For example, the second state may include an irregular heartbeat just as the first state did in some embodiments. In some embodiments, the excluded at least one cardiac cycle of the second sequence of consecutive cardiac cycles occurs between at least two cardiac cycles of the some, but not all, of the second sequence of consecutive cardiac cycles. In some embodiments, the data processing device system (e.g., 110, 310) is configured at least by the program at least to cause (c) which includes both of (a) and (b) discussed above.

According to some embodiments, the data processing device system (e.g., 110, 310) is configured at least by the program at least to cause (a) in association with the first state (e.g., the example first state of FIG. 7C), the first pulsed field ablation transducer (e.g., 220, 306, 406) to deliver a respective subset of the plurality of first high voltage pulses during each cardiac cycle of the first sequence of consecutive cardiac cycles (e.g., cardiac cycles 720c, 722c). In some embodiments, the data processing device system (e.g., 110, 310) is configured at least by the program at least to cause (b) in association with the second state (e.g., akin to the state of FIG. 7E), the first pulsed field ablation transducer (e.g., 220, 306, 406) to deliver a respective subset of the plurality of second high voltage pulses during each cardiac cycle of some, but not all, of the cardiac cycles of the second sequence of consecutive cardiac cycles (e.g., pulse subsets delivered in cardiac cycles 720e, 722e). According to some embodiments, the some, but not all, of the second sequence of consecutive cardiac cycles excludes at least one cardiac cycle (e.g., cardiac cycle 721e) of the second sequence of consecutive cardiac cycles during which no pulsed field ablation energy is delivered by the first pulsed field ablation transducer (e.g., 220, 306, 406). In some embodiments, the excluded at least one cardiac cycle of the second sequence of consecutive cardiac cycles occurs between at least two cardiac cycles of the some, but not all, of the second sequence of consecutive cardiac cycles. For instance, although it may be considered in FIG. 7E that the second sequence of consecutive cardiac cycles includes cardiac cycles 721e, 722e, cardiac cycle 720e may also be considered part of the second sequence of consecutive cardiac cycles, according to some embodiments, such that, e.g., the irregular cardiac cycle 721e is between cardiac cycles 720e, 722e.

In some embodiments, the number of first high voltage pulses in each of at least one of the respective subsets of the plurality of first high voltage pulses is the same as the number of second high voltage pulses in each of at least one of the respective subsets of the plurality of second high voltage pulses. For example, pulse train 732c has a same number of pulses as pulse train 732e (shown in plot 716e), according to some embodiments. In some embodiments, the number of first high voltage pulses in each of at least one of the respective subsets of the plurality of first high voltage pulses is different than the number of second high voltage pulses in each of at least one of the respective subsets of the plurality of second high voltage pulses. For example, pulse train 732c has a different number of pulses than pulse train 734e, according to some embodiments. In some embodiments, the number of first high voltage pulses in each of the respective subsets of the plurality of first high voltage pulses is the same as the number of second high voltage pulses in each of the respective subsets of the plurality of second high voltage pulses. For example, each of pulse train 734c in FIG. 7C and pulse train 734e in FIG. 7E could instead have the same number of pulses as each of pulse trains 732c and 732e, according to some embodiments. In some embodiments, the number of first high voltage pulses in each of the respective subsets of the plurality of first high voltage pulses is different than the number of second high voltage pulses in each of the respective subsets of the plurality of second high voltage pulses. For example, the pulse trains 732c, 734c, 732e, 734e may all have different numbers of pulses, according to some embodiments, which may be a result of controlling an overall average PFA energy delivered over at least a portion of a procedure in some embodiments.

In some embodiments, the first high voltage pulses in each of at least one of the respective subsets of the plurality of first high voltage pulses are configured to cumulatively deliver first energy during the respective cardiac cycle of the first sequence of consecutive cardiac cycles, and the second high voltage pulses in each of at least one of the respective subsets of the plurality of second high voltage pulses are configured to cumulatively deliver second energy during the respective cardiac cycle of the second sequence of consecutive cardiac cycles, the second energy being the same as the first energy, according to some embodiments. For example, the first energy 737c in FIG. 7C may be the same as second energy 739e in FIG. 7E, in some embodiments. In some embodiments, the first high voltage pulses in each of at least one of the respective subsets of the plurality of first high voltage pulses are configured to cumulatively deliver first energy during the respective cardiac cycle of the first sequence of consecutive cardiac cycles, and the second high voltage pulses in each of at least one of the respective subsets of the plurality of second high voltage pulses are configured to cumulatively deliver second energy during the respective cardiac cycle of the second sequence of consecutive cardiac cycles, the second energy different than the first energy, according to some embodiments. For example, the first energy 737c in FIG. 7C may be different than second energy 739e in FIG. 7E, in some embodiments.

In some embodiments, the first high voltage pulses of the plurality of first high voltage pulses are configured to cumulatively deliver first energy (e.g., a sum of cumulative energies 737c, 739c in FIG. 7C in some embodiments) throughout the first sequence of consecutive cardiac cycles (e.g., cardiac cycles 720c, 722c in some embodiments), and the second high voltage pulses of the plurality of second high voltage pulses are configured to cumulatively deliver second energy (e.g., a sum of cumulative energies 737e, 739e shown in plot 717e in FIG. 7E) during the second sequence of consecutive cardiac cycles (e.g., cardiac cycles 720e, 721e, 722e in some embodiments). In some embodiments, a third particular ratio of this first energy to the total number of cardiac cycles (e.g., two cycles in this example) in the first sequence of consecutive cardiac cycles is different than a fourth particular ratio of this second energy to the total number of cardiac cycles (e.g., three cycles in this example) in the second sequence of consecutive cardiac cycles. Although the examples of FIG. 7C and FIG. 7E (and others of FIG. 7) show certain numbers of cardiac cycles, high voltage pulses, and pulse trains, and certain correspondences between pulse trains and cardiac cycles, it is understood that these examples are not limiting and other embodiments may have different numbers and correspondences.

Differences between the third particular ratio of the first energy to the total number of cardiac cycles in the first sequence of consecutive cardiac cycles and the fourth particular ratio of the second energy to the total number of cardiac cycles in the second sequence of consecutive cardiac cycles may be motivated for different reasons. For example, in some embodiments in which cardiac cycles are “skipped” in terms of high voltage pulse delivery (for example, as described above or otherwise in this disclosure), differences between the third particular ratio of the first energy to the total number of cardiac cycles in the first sequence of consecutive cardiac cycles and the fourth particular ratio of the second energy to the total number of cardiac cycles may result. In various embodiments, average power delivered by the high voltage pulses is maintained regardless of these differences between the third and fourth particular ratios.

FIG. 6C illustrates a programmed configuration 620 of a data processing device system (e.g., 110, 310), according to some embodiments of the present invention. In some embodiments in which the programmed configuration illustrated in FIG. 6C actually is executed at least in part by the data processing device system, such actual execution may be considered a respective method executed by the data processing device system. In this regard, reference numeral 620 and FIG. 6C may be considered to represent one or more methods in some embodiments and, for ease of communication, one or more methods 620 may be referred to at times simply as method 620. The blocks shown in FIG. 6C may be associated with computer-executable instructions of a program that configures the data processing device system to perform the actions described by the respective blocks. According to various embodiments, not all of the actions or blocks shown in FIG. 6C are required, and different orderings of the actions or blocks shown in FIG. 6C may exist. In this regard, in some embodiments, a subset of the blocks shown in FIG. 6C or additional blocks may exist. In some embodiments, a different sequence of various ones of the blocks in FIG. 6C or actions described therein may exist.

In some embodiments, a memory device system (e.g., 130, 330 or a computer-readable medium system) stores the program represented by FIG. 6C, and, in some embodiments, the memory device system is communicatively connected to the data processing device system as a configuration thereof. In this regard, in various example embodiments, a memory device system (e.g., memory device system 130, 330) is communicatively connected to a data processing device system (e.g., 110, 310) and stores a program executable by the data processing device system to cause the data processing device system to execute various actions described by, or otherwise associated with, the blocks illustrated in FIG. 6C for performance of some, or all, of method 620 via interaction with at least, for example, a transducer-based device (e.g., PFA devices 200A, 300A, or 400A). In these various embodiments, the program may include instructions configured to perform, or cause to be performed, various ones of the block actions described by, or otherwise associated with, one or more or all of the blocks illustrated in FIG. 6C for performance of some or all of method 620.

In FIG. 6C, according to some embodiments, block 622 represents a configuration of the data processing device system (e.g., 110, 310) at least to cause delivery via an input-output device system (e.g., 120, 320) and via a first pulsed field ablation transducer (e.g., 220, 306, 406) located on a catheter device, of a respective high voltage pulse train during each respective cardiac cycle of a plurality of cardiac cycles (e.g., as shown by the example of FIG. 7C). In this regard, such delivery may be executed differently in different embodiments, as shown at least by blocks 622a, 622b in FIG. 6C, according to some embodiments. Further in this regard, either or both of the blocks 622a, 622b may occur or be executed, and in the case where both occur or are executed, they may occur or be executed in any order, as illustrated by the double-headed broken line arrow in FIG. 6C between blocks 622a and 622b, according to various embodiments.

According to some embodiments, each respective high voltage pulse train referred to per block 622 defines a plurality of high voltage pulses, each respective high voltage pulse train configured to cause pulsed field ablation of tissue. In some embodiments, each high voltage pulse is configured to cause pulsed field ablation of tissue. According to various embodiments, delivery of each respective high voltage pulse train is caused (e.g., by the data processing device system (e.g., 110, 310)) to occur only during a particular time interval (e.g., particular time interval 729c, 731c) in the respective cardiac cycle. In some embodiments, each respective high voltage pulse train is deliverable only during a particular time interval in the respective cardiac cycle. According to some embodiments, the respective cardiac cycles of the plurality of cardiac cycles include at least a first cardiac cycle (e.g., cardiac cycle 720c) and a second cardiac cycle (e.g., cardiac cycle 722c). It is noted that the second cardiac cycle need not immediately succeed the first cardiac cycle according to some embodiments. According to some embodiments, the particular time intervals (e.g., particular time intervals 729c, 731c) in the first cardiac cycle and the second cardiac cycle during which respective pulse trains are deliverable may be configured such that at first ratio of the duration of the particular time interval in the first cardiac cycle to the duration of the first cardiac cycle is different than a second ratio of the duration of the particular time interval in the second cardiac cycle to the duration of the second cardiac cycle. For instance, in the example of FIG. 7C, the differences in durations 724c, 726c of respective cardiac cycles 720c, 722c may lead to such ratio differences in some embodiments.

In some embodiments, the respective high voltage pulse train which the data processing device system (e.g., 110, 310) is configured to cause delivery of during the first cardiac cycle is configured to have a first particular number (e.g., first particular number 736c) of high voltage pulses, and the respective high voltage pulse train which the data processing device system (e.g., 110, 310) is configured to cause delivery of during the second cardiac cycle is configured to have a second particular number (e.g., second particular number 738c) of high voltage pulses. In some embodiments, the second particular number of high voltage pulses is different than the first particular number of high voltage pulses. In some embodiments, the first ratio is less than the second ratio, and the first particular number of high voltage pulses is greater than the second particular number of high voltage pulses. In some embodiments, the duration (e.g., duration 724c) of the first cardiac cycle is longer than the duration (e.g., duration 726c) of the second cardiac cycle, and the first particular number of high voltage pulses is greater than the second particular number of high voltage pulses.

Different combinations of particular time interval durations and respective cardiac cycles may be employed by the first ratio and the second ratio, according to some embodiments. In some embodiments, the duration of the particular time interval (e.g., particular time interval 729c) in the first cardiac cycle is the same as the duration of the particular time interval (e.g., particular time interval 731c) in the second cardiac cycle. In at least some embodiments in which the particular time intervals in the first and second cardiac cycles have same durations, different first and second ratios may be provided when the durations of the first cardiac cycle and the second cardiac cycles are different. In some embodiments, the duration (e.g., duration 724c) of the first cardiac cycle is different than the duration (e.g., duration 726c) of the second cardiac cycle. According to some embodiments, a duration of each particular time interval of the particular time intervals in the first cardiac cycle and the second cardiac cycle is shorter than a duration of the respective cardiac cycle. For instance, although the example of FIG. 7C shows fixed durations of particular time intervals 729c, 731c triggered off of a particular cardiac event in the QRS complex of the respective cardiac cycles 720c, 722c, other embodiments may not have fixed durations. For example, such particular time intervals may instead be of variable duration.

According to some embodiments, per block 622a in FIG. 6C, the high voltage pulses of the respective high voltage pulse train (e.g., high voltage pulse train 732c) which the data processing device system (e.g., 110, 310) is configured to cause delivery of during the first cardiac cycle (e.g., cardiac cycle 720c) are configured to cumulatively deliver first energy (e.g., first energy 737c) during the particular time interval in the first cardiac cycle. According to some embodiments, the high voltage pulses of the respective high voltage pulse train (e.g., high voltage pulse train 734c) which the data processing device system (e.g., 110, 310) is configured to cause delivery of during the second cardiac cycle (e.g., cardiac cycle 722c) are configured to cumulatively deliver second energy (e.g., second energy 739c) during the particular time interval in the second cardiac cycle. In various embodiment, the second energy is different than the first energy. In some embodiments, the first ratio of the duration of the particular time interval in the first cardiac cycle to the duration of the first cardiac cycle is less than the second ratio of the duration of the particular time interval in the second cardiac cycle to the duration of the second cardiac cycle, and the first energy is greater than the second energy.

For example, such embodiments may alleviate the Joule heating effects described above or otherwise herein in some cases in which the first cardiac cycle has a longer duration than the duration of the second cardiac cycle (e.g., the first cardiac cycle is associated with a heart rate that is relatively slower than a heart rate associated with the second cardiac cycle). In some embodiments, the first energy is greater than the second energy. In some embodiments, the first ratio is less than the second ratio, and the first energy is greater than the second energy. In some embodiments, the duration of the first cardiac cycle is longer than the duration of the second cardiac cycle, and the first energy is greater than the second energy. In some embodiments, one of (a) a ratio of the first energy to the duration (e.g., actual or predicted in some embodiments) of the first cardiac cycle and (b) a ratio of the second energy to the duration (e.g., actual or predicted in some embodiments) of the second cardiac cycle is within a particular percentage of the other of (a) and (b). In some embodiments, the particular percentage is 5%. In some embodiments, the particular percentage is 10%. In some embodiments, the particular percentage is 15%. In some embodiments, the particular percentage is 20%.

In some embodiments, the respective high voltage pulse train which the data processing device system (e.g., 110, 310) is configured to cause delivery of during the first cardiac cycle is configured to cause delivery of a first average power during the first cardiac cycle (e.g., cardiac cycle 720c), and the respective high voltage pulse train which the data processing device system is configured to cause delivery of during the second cardiac cycle (e.g., cardiac cycle 722c) is configured to cause delivery of a second average power that maintains the first average power. In some embodiments, the second average power maintains the first average power by being within 5% of the first average power. In some embodiments, the second average power maintains the first average power by being within 10% of the first average power. In some embodiments, the second average power maintains the first average power by being within 15% of the first average power. In some embodiments, the second average power maintains the first average power by being within 20% of the first average power.

In some embodiments, a duration of the particular time interval in the respective cardiac cycle of each of the plurality of cardiac cycles is less than or shorter than a duration of the respective cardiac cycle. For instance, the particular time intervals 729c, 731c are shorter than the durations 724c, 726c of the respective cardiac cycles 720c, 722c in the example of FIG. 7C. In some embodiments, a duration of least one of the particular time intervals is shorter than a duration of the respective cardiac cycle. In some embodiments, the particular time interval in the respective cardiac cycle is less than the entirety of the respective cardiac cycle. In some embodiments, no pulse configured to cause PFA is delivered during the respective cardiac cycle outside of the particular time interval in the respective cardiac cycle. For example, no pulse configured to cause PFA is delivered within the cardiac cycle 720c outside of the particular time interval 729c in the example of FIG. 7C, in some embodiments. In some embodiments, no pulse configured to cause PFA is delivered by at least the first pulsed field ablation transducer during the respective cardiac cycle outside of the particular time interval in the respective cardiac cycle, the first pulsed field ablation transducer being one that delivers a respective high voltage PFA pulse train during the particular time interval during the respective cardiac cycle.

In some embodiments, the particular time interval in the first cardiac cycle has a first determined temporal relationship with a particular cardiac event in the first cardiac cycle, and the particular time interval in the second cardiac cycle has a second determined temporal relationship with a particular cardiac event in the second cardiac cycle. For example, as discussed above, the particular time intervals may have a determined temporal relationship with a particular portion of the QRS complex of the respective cardiac cycles, according to some embodiments. In some embodiments, the first determined temporal relationship is the same as the second determined temporal relationship. In some embodiments, the particular time interval in the first cardiac cycle occurs during a refractory period (e.g., refractory period 728c) in the first cardiac cycle. In some embodiments, the particular time interval in the second cardiac cycle occurs during a refractory period (e.g., refractory period 730c) in the second cardiac cycle.

In some embodiments, the respective high voltage pulse train (e.g., high voltage pulse train 732c) which the data processing device system is configured to cause delivery of during the first cardiac cycle (e.g., cardiac cycle 720c) is configured to have a first inter-pulse spacing (e.g., inter-pulse spacing 744c) between adjacent high voltage pulses in the respective high voltage pulse train. In some embodiments, the respective high voltage pulse train (e.g., high voltage pulse train 734c) which the data processing device system is configured to cause delivery of during the second cardiac cycle (e.g., cardiac cycle 722c) is configured to have a second inter-pulse spacing (e.g., inter-pulse spacing 746c) between adjacent high voltage pulse in the respective high voltage pulse train. In some embodiments, the second inter-pulse spacing (e.g., inter-pulse spacing 746c) is different than the first inter-pulse spacing (e.g., inter-pulse spacing 744c).

In some embodiments, each of at least one high voltage pulse in the respective high voltage pulse train (e.g., high voltage pulse train 732c) which the data processing device system is configured to cause delivery of during the first cardiac cycle (e.g., cardiac cycle 720c) is configured to deliver a respective first amount of pulse energy (e.g., pulse energy symbolized by reference 740c). In some embodiments, each of at least one high voltage pulse in the respective high voltage pulse train (e.g., high voltage pulse train 734c) which the data processing device system is configured to cause delivery of during the second cardiac cycle (e.g., cardiac cycle 722c) is configured to deliver a respective second amount of pulse energy (e.g., pulse energy symbolized by reference 742c). In some embodiments, each high voltage pulse in each high voltage pulse train of the plurality of high voltage pulse trains (e.g., high voltage pulse trains 732c, 734c) is a high voltage pulse of at least 150 volts. In some embodiments, each respective second amount of pulse energy (e.g., pulse energy symbolized by reference 742c) is different than each respective first amount of pulse energy (e.g., pulse energy symbolized by reference 740c).

In some embodiments, each of at least one high voltage pulse in the respective high voltage pulse train (e.g., high voltage pulse train 732c) which the data processing device system is configured to cause delivery of during the first cardiac cycle (e.g., cardiac cycle 720c) is configured to have a respective first pulse shape (e.g., a square wave pulse in FIG. 7C). In some embodiments, each of at least one high voltage pulse in the respective high voltage pulse train (e.g., high voltage pulse train 734c) which the data processing device system is configured to cause delivery of during the second cardiac cycle (e.g., cardiac cycle 722c) is configured to have a respective second pulse shape (e.g., a square wave pulse in FIG. 7C). In some embodiment, each respective second pulse shape is different than each respective first pulse shape. For example, although the pulses of pulse trains 732c, 734c in FIG. 7C all are square wave pulses, differing pulse shapes may be provided, such as square wave pulses and triangular wave pulses, by way of non-limiting example. Such control of pulse shape may be utilized, e.g., to control energy delivery, among other things, according to various embodiments.

In some embodiments, per block 622b, the data processing device system (e.g., 110, 310) is configured at least by the program at least to cause, in response to reception of information indicative of a particular cardiac event occurring in each of at least some of the plurality of cardiac cycles, a change in at least one high voltage pulse train parameter to cause at least two respective high voltage pulse trains to be different from each other. For example, with reference to FIG. 7C, the data processing device system may receive information (e.g., electrocardiogram-based information) indicative of a cardiac event occurring in each of at least cardiac cycle 720c and cardiac cycle 722c, which the data processing device system may process to cause delivery of the first high voltage pulse train 732c and second high voltage pulse train 734c, where at least one high voltage pulse train parameter is different between the pulse trains 732c, 734c. In some embodiments, each of the at least some of the plurality of cardiac cycles occurs prior to at least one of the respective cardiac cycles associated with the at least two respective high voltage pulse trains. For example, each of the at least some of the plurality of cardiac cycles may be or include cardiac cycle 718c, which occurs before at least the cardiac cycles 720c, 722c associated with the high voltage pulse trains 732c, 734c, according to some embodiments. In this regard, in some embodiments, even though a pulse train is not shown as being delivered during cardiac cycle 718c, such cardiac cycle may include a delivered pulse train. In some embodiments, the at least some of the plurality of cardiac cycles may be or include a number of cardiac cycles (not shown in FIG. 7C) occurring between cardiac cycles 720c, 722c, such that the number of cardiac cycles may gradually reduce in duration, accounting for the change between durations 724c, 726c of cardiac cycles 720c, 722c.

According to some embodiments, the information indicative of a particular cardiac event occurring in each of at least some of the plurality of cardiac cycles indicates at least an occurrence of the particular cardiac event occurring in the respective cardiac cycle associated with one of the at least two respective high voltage pulse trains (e.g., the two respective high voltage pulse trains 732c, 734c in some embodiments). In some embodiments, the information indicative of the particular cardiac event occurring in each of at least some of the plurality of cardiac cycles indicates an occurrence of the particular cardiac event in each cardiac cycle of a group of consecutively occurring cardiac cycles of the plurality of cardiac cycles. In some embodiments, the particular cardiac event is at least part of a QRS complex. In some embodiments, the particular cardiac event is at least part of a P wave. In some embodiments, the particular cardiac event is a cardiac pulse caused by a pacing signal deliverable to a patient by a pacing device system.

In some embodiments, the data processing device system (e.g., 110, 310) may be configured at least by the program at least to determine one or more cardiac cycle durations based at least on the indicated occurrence of the particular cardiac event in each cardiac cycle of the group of consecutively occurring cardiac cycles of the plurality of cardiac cycles. For example, a cardiac cycle duration may be determined based on a time interval between an indicated initial occurrence of the particular cardiac event and an indicated reoccurrence of the particular cardiac event immediately after the initial occurrence. Various methods of determining a cardiac cycle duration are described above or otherwise in this disclosure.

In some embodiments, the data processing device system (e.g., 110, 310) is configured at least by the program to cause the change in the at least one high voltage pulse train parameter based at least on the determined one or more cardiac cycle durations. For example, the at least one high voltage pulse train parameter may be changed in accordance with a determined cardiac cycle duration in a manner the same or similar as described above or otherwise in this disclosure. In this regard, in some embodiments, each high voltage pulse in the each respective high voltage pulse train is configured to deliver a respective amount of pulse energy (e.g., first amount of pulse energy 740c for pulse train 732c and second amount of pulse energy 742c for pulse train 734c), and the changing in at least one high voltage pulse train parameter may be configured to cause a change in the respective amount of pulse energy that is delivered by each of at least one high voltage pulse in at least one high voltage pulse train of the at least two of the respective high voltage pulse trains. For example, the first amount of pulse energy 740c and the second amount of pulse energy 742c may be caused by the data processing device system to be different due at least to differences in the cardiac cycles 718c, 720c, according to some embodiments.

In some embodiments, the changing in at least one high voltage pulse train parameter may include a change in the number of high voltage pulses in at least one high voltage pulse train of the at least two of the respective high voltage pulse trains, as shown, for example, by the differences between number of pulses 736c and number of pulses 738c in FIG. 7C. In some embodiments, the changing in at least one high voltage pulse train parameter may include a change in an inter-pulse spacing between the high voltage pulses in at least one high voltage pulse train of the at least two of the respective high voltage pulse trains, as shown, for example, by the differences in inter-pulse spacings 744c, 746c in FIG. 7C. In some embodiments, the changing in at least one high voltage pulse train parameter may include a change in a pulse shape (e.g., per the discussion with respect to at least FIG. 9) in each of one or more high voltage pulses in at least one high voltage pulse train of the at least two of the respective high voltage pulse trains.

In some embodiments, the reception of the information indicative of a particular cardiac event (occurring in each of at least some of the plurality of cardiac cycles) may indicate a transition from a first cardiac cycle set to a subsequent second cardiac cycle set, and the data processing device system (e.g., 110, 310) is configured at least by the program, in response to the reception of the information indicative of the particular cardiac event, to cause at least one high voltage pulse train parameter employed by a second high voltage pulse train that is to be delivered during a cardiac cycle (e.g., cardiac cycle 722c) of the second cardiac cycle set to be different than the corresponding at least one high voltage pulse train parameter employed by a first high voltage pulse train delivered during a cardiac cycle (e.g., cardiac cycle 720c) of the first cardiac cycle set, the at least two of the respective high voltage pulse trains including the first high voltage pulse train and the second high voltage pulse train. In this regard, in some embodiments, one or more pulse train parameters may be adjusted or modified on an individual cardiac cycle basis (e.g., when the first cardiac cycle set includes a single cardiac cycle, according to some embodiments), or, in some embodiments, one or more pulse train parameters may be adjusted or modified over a longer span of time on a multiple cardiac cycle basis (e.g., when the first cardiac cycle set includes multiple cardiac cycles), e.g., depending on the energy delivery goals and cardiac cycle characteristics experienced in a particular medical procedure. In some embodiments, one or more pulse train parameters may be adjusted or modified over a longer span of time on a multiple cardiac cycle basis (e.g., when the first cardiac cycle set includes multiple cardiac cycles) while the first pulsed field ablation transducer is in a same bodily cavity. In some embodiments, one or more cardiac cycles may be between the first and second cardiac cycle sets, allowing longer or earlier energy delivery and cardiac cycle characteristic trends to impact present or future energy delivery. This may provide more accurate energy delivery and more control over the medical procedure.

In some embodiments, the data processing device system (e.g., 110, 310) is configured at least by the program, in response to the reception of the information indicative of the particular cardiac event, to cause an initiation of a delivery of a particular pulse train (e.g., first high voltage pulse train 732c) of one of the at least two (e.g., first high voltage pulse train 732c and second high voltage pulse train 734c) of the respective high voltage pulse trains. In some embodiments, the initiation of the delivery of the particular pulse train of one of the at least two of the respective high voltage pulse trains may be gated to the indicated particular cardiac event, e.g., at least part of a QRS complex, as discussed above or otherwise herein. According to various embodiments, the initiation of the delivery of the particular pulse train of one of the at least two of the respective high voltage pulse trains is an initiation of a pulse train of the at least two of the respective high voltage pulse trains that has at least one different pulse train parameter than another pulse train of the at least two of the respective high voltage pulse trains. For example, the pulse trains 732c, 734c have various different parameters as discussed above or otherwise herein. The indicated particular cardiac event may correspond to a particular portion of a QRS complex, in some embodiments. In some embodiments, a start of the particular time interval in a particular cardiac cycle may be defined in accordance with a pre-determined or determined temporal relationship with a detected particular cardiac event in the particular cardiac cycle, as discussed above or otherwise herein.

According to some embodiments, a duration of each particular time interval is shorter than a duration of the respective cardiac cycle, e.g., as shown by the particular time intervals of 729c, 731c being shorter in duration than their respective cardiac cycles 720c, 722c. In some embodiments, the particular time intervals in the respective cardiac cycles associated with the at least two of the respective high voltage pulse trains have a same duration. In some embodiments, the respective cardiac cycles associated with the at least two of the respective high voltage pulse trains have different durations, e.g., as shown by the different durations 724c, 726c of the respective cardiac cycles 720c, 722c. In some embodiments, the at least two of the respective high voltage pulse trains include a first high voltage pulse train (e.g., first high voltage pulse train 732c) and a second high voltage pulse train (e.g., second high voltage pulse train 734c), and a first ratio of a duration of the particular time interval (e.g., particular time interval 729c) in the respective cardiac cycle (e.g., cardiac cycle 720c) associated with the first high voltage pulse train (e.g., high voltage pulse train 732c) to a duration of the respective cardiac cycle associated with the first high voltage pulse train is different than a second ratio of a duration of the particular time interval (e.g., a particular time interval 731c) in the respective cardiac cycle (e.g., cardiac cycle 722c) associated with the second high voltage pulse train (e.g., high voltage pulse train 734c) to a duration of the respective cardiac cycle associated with the second high voltage pulse train. In some embodiments, the particular time interval in the respective cardiac cycle associated with each of the at least two of the respective high voltage pulse trains occurs during a refractory period (e.g., refractory period 728c and refractory period 730c) in each of the respective cardiac cycles associated with each of the at least two of the respective high voltage pulse trains.

In some embodiments, the at least two of the respective high voltage pulse trains include a first high voltage pulse train (e.g., high voltage pulse train 732c) and a second high voltage pulse train (e.g., high voltage pulse train 734c). In some embodiments, the high voltage pulses of the first high voltage pulse train (e.g., high voltage pulse train 732c) are configured to cumulatively deliver a first energy (e.g., first energy 737c) during the particular time interval (e.g., particular time interval 729c) of the respective cardiac cycle (e.g., cardiac cycle 720c), and the high voltage pulses of the second high voltage pulse train (e.g., high voltage pulse train 734c) are configured to cumulatively deliver a second energy (e.g., second energy 739c) during the particular time interval (e.g., particular time interval 731c) of the respective cardiac cycle (e.g., cardiac cycle 722c). In some embodiments, the second energy is different than the first energy. In some embodiments, a first ratio of a duration of the particular time interval (e.g., particular time interval 729c) in the respective cardiac cycle (e.g., cardiac cycle 720c) associated with the first high voltage pulse train (e.g., first high voltage pulse train 732c) to a duration (e.g., duration 724c) of the respective cardiac cycle associated with the first high voltage pulse train is different than a second ratio of a duration of the particular time interval (e.g., particular time interval 731c) in the respective cardiac cycle (e.g., cardiac cycle 722c) associated with the second high voltage pulse train (e.g., second high voltage pulse train 734c) to a duration (e.g., duration 726c) of the respective cardiac cycle associated with the second high voltage pulse train. In some embodiments, the first energy (e.g., first energy 737c) is greater than the second energy (e.g., second energy 739c), and the first ratio is less than the second ratio. In some embodiments, the particular time interval (e.g., particular time interval 729c) in the respective cardiac cycle associated with the first high voltage pulse train has a first temporal relationship with an occurrence of the particular cardiac event (e.g., a particular portion of a QRS complex) in the respective cardiac cycle associated with the first high voltage pulse train, and the particular time interval (e.g., particular time interval 731c) in the respective cardiac cycle associated with the second high voltage pulse train has a second temporal relationship with an occurrence of the particular cardiac event in the respective cardiac cycle associated with the second high voltage pulse train, the first temporal relationship and the second temporal relationship being a same temporal relationship, according to some embodiments.

In some embodiments, a duration of the particular time interval in the respective cardiac cycle associated with the first high voltage pulse train is shorter than a duration of the respective cardiac cycle associated with the first high voltage pulse train, and a duration of the particular time interval in the respective cardiac cycle associated with the second high voltage pulse train is shorter than a duration of the respective cardiac cycle associated with the second high voltage pulse train. For example, the particular time intervals 729c, 731c are shorter than their respective cardiac cycles 720c, 722c in the example of FIG. 7C. In some embodiments, the duration of the respective cardiac cycle associated with the first high voltage pulse train is different than the duration of the respective cardiac cycle associated with the second high voltage pulse train. For instance, the duration 724c of the cardiac cycle 720c is different than the duration 726c of the cardiac cycle 722c in the example of FIG. 7C. In some embodiments, the duration of the particular time interval (e.g., particular time interval 729c) in the respective cardiac cycle associated with the first high voltage pulse train and the duration of the particular time interval (e.g., particular time interval 731c) in the respective cardiac cycle associated with the second high voltage pulse train are the same. In some embodiments, the first energy (e.g., first energy 737c) is greater than the second energy (e.g., second energy 739c), and the duration (e.g., duration 724c) of the respective cardiac cycle associated with the first high voltage pulse train is greater than the duration (e.g., duration 726c) of the respective cardiac cycle associated with the second high voltage pulse train.

In some embodiments, one of a first ratio of the first energy (e.g., first energy 737c) to the (e.g., actual or predicted) duration (e.g., duration 724c) of the respective cardiac cycle (e.g., cardiac cycle 720c) associated with the first high voltage pulse train (e.g., high voltage pulse train 732c) and a second ratio of the second energy (e.g., second energy 739c) to the (e.g., actual or predicted) duration (e.g., duration 726c) of the respective cardiac cycle (e.g., cardiac cycle 722c) associated with the second high voltage pulse train (e.g., high voltage pulse train 734c) is within a particular percentage of the other of the first ratio and the second ratio. In some embodiments, the particular percentage is 5%. In some embodiments, the particular percentage is 10%. In some embodiments, the particular percentage is 15%. In some embodiments, the particular percentage is 20%.

In some embodiments, the data processing device system (e.g., 110, 310) is configured at least by the program at least to cause the first high voltage pulse train to deliver a first average power during the respective cardiac cycle (e.g., cardiac cycle 720c), and cause the second high voltage pulse train to deliver a second average power during the respective cardiac cycle (e.g., cardiac cycle 722c), the second average power configured to maintain the first average power. In some embodiments, the second average power maintains the first average power by being within 5% of the first average power. In some embodiments, the second average power maintains the first average power by being within 10% of the first average power. In some embodiments, the second average power maintains the first average power by being within 15% of the first average power. In some embodiments, the second average power maintains the first average power by being within 20% of the first average power.

According to various embodiments, in which temperature elevation is occurring during PFA, decreasing the energy delivered per pulse train (or per pulse in some embodiments) in a heartbeat-dependent manner may also be used to control total power delivery in a predictable manner as tissue warms. In this way, according to various embodiments, one or more pulse train parameters may be adjusted over the delivery of therapy in response to the warming of the tissue. In some embodiments, rather than, or in addition to, targeting a predefined average power, various PFA high voltage pulse train parameters (e.g., the pulse rate per heartbeat, pulse amplitude or voltage, pulse duration, pulse shape, or various combinations thereof) may also be varied in response to information responsive to temperature changes in tissue portions undergoing pulsed field ablation. In some embodiments, rather than, or in addition to, targeting a predefined average power, various PFA high voltage pulse train parameters (e.g., the pulse rate per heartbeat, pulse amplitude or voltage, pulse duration, pulse shape, or various combinations thereof) may also be varied in response to information responsive to temperature information provided by one or more transducers. In some embodiments, rather than, or in addition to, targeting a predefined average power, various PFA high voltage pulse train parameters (e.g., the pulse rate per heartbeat, pulse amplitude or voltage, pulse duration, pulse shape, or various combinations thereof) may also be varied in response to information responsive to temperature information provided by one or more PFA transducers (e.g., 220, 306, 406). According to various embodiments, various PFA pulse train parameters may be controlled to allow temperature information (e.g., temperature information or a proxy for temperature information) measured by a one or more transducers to reach one or more target levels. According to various embodiments, the measured temperature is a function of the balance between PFA power being electrically delivered to the contacting tissue and the removal of thermal energy by conduction through the tissue as well as convective loss to circulating blood. According to various embodiments, temperature control using PFA high voltage pulse train parameters may provide a more direct approach to controlling the blood/tissue temperature, providing the measured temperature is a suitable proxy for the maximum tissue or blood temperature. Various temperature sensing transducers may be used according to various embodiments. In some embodiments, the temperature sensing transducers are configured for internal placement within a patient. For example, temperature sensing transducers may be transported by a catheter to a desired location in a patient body. In some embodiments, the temperature sensing transducers are separate from various PFA transducers employed according to some embodiments. In some embodiments, the one or more PFA transducers may also include temperature sensing capabilities. For example, a PFA transducer, such as PFA transducer 220, 306, 406 includes, according to some embodiments, a temperature sensor 408.

FIG. 6D illustrates a programmed configuration 630 of a data processing device system (e.g., 110, 310), according to some embodiments of the present invention. In some embodiments in which the programmed configuration illustrated in FIG. 6D actually is executed at least in part by the data processing device system, such actual execution may be considered a respective method executed by the data processing device system. In this regard, reference numeral 630 and FIG. 6D may be considered to represent one or more methods in some embodiments and, for ease of communication, one or more methods 630 may be referred to at times simply as method 630. The blocks shown in FIG. 6D may be associated with computer-executable instructions of a program that configures the data processing device system to perform the actions described by the respective blocks. According to various embodiments, not all of the actions or blocks shown in FIG. 6D are required, and different orderings of the actions or blocks shown in FIG. 6D may exist. In this regard, in some embodiments, a subset of the blocks shown in FIG. 6D or additional blocks may exist. In some embodiments, a different sequence of various ones of the blocks in FIG. 6D or actions described therein may exist.

In some embodiments, a memory device system (e.g., 130, 330 or a computer-readable medium system) stores the program represented by FIG. 6D, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions described by or otherwise associated with the blocks illustrated in FIG. 6D for performance of some or all of method 630 via interaction with at least, for example, a transducer-based device (e.g., PFA devices 200A, 300A, or 400A). In these various embodiments, the program may include instructions configured to perform, or cause to be performed, various ones of the block actions described by or otherwise associated with one or more or all of the blocks illustrated in FIG. 6D for performance of some or all of method 630.

In FIG. 6D, according to some embodiments, block 632 represents a configuration of the data processing device system (e.g., data processing device system 110 or 310) to cause, via the input-output device system (e.g., 110, 310), each of at least a first transducer of a plurality of transducers (e.g., 220, 306, 406) located on a catheter device, to deliver a respective first high voltage pulse train (e.g., high voltage pulse train 732c, FIG. 7C) of a first high voltage pulse train set during a first cardiac cycle (e.g., cardiac cycle 720c, FIG. 7C). According to various embodiments, each high voltage pulse train of the first high voltage pulse train set is configured to cause pulsed field ablation of tissue. According to various embodiments, each of the plurality of transducers (e.g., 220, 306, 406) is deliverable by the catheter device to a respective location within a patient (e.g., a respective location in a bodily opening or a respective location in a bodily organ).

In FIG. 6D, according to some embodiments, block 634 represents a configuration of the data processing device system (e.g., data processing device system 110 or 310) to cause reception, via the input-output device system (e.g., 110, 310), of information indicative of temperature at least proximate a second transducer of the plurality of transducers during or after delivery of at least part of the first high voltage pulse train set. In some embodiments, the second transducer is the first transducer, such that, e.g., the same transducer that performs PFA has temperature sensed adjacent it. In some embodiments, the same transducer is configured to perform PFA and to sense temperature. In some embodiments, the second transducer is other than the first transducer, such that, e.g., temperature sensed adjacent the second transducer may be employed to control PFA via the first transducer. In some embodiments, the first transducer is configured at least to perform PFA, and the second transducer is configured to at least sense temperature, such that, e.g., the second transducer may sense temperature adjacent the first transducer in some embodiments. In some embodiments, the first transducer and the second transducer are adjacently located on the catheter. In some embodiments, the second transducer is a PFA transducer, and the data processing device system may be configured to control the second transducer to not deliver pulsed field ablation energy during the delivery of at least part of the first high voltage pulse train set. For instance, in some embodiments where temperature is sensed adjacent or by the second transducer to control PFA executed by the first transducer, it may be preferable to have the second transducer not deliver PFA energy while the first transducer performs PFA. In some embodiments, the second transducer is a PFA transducer, and the data processing device system may be configured to control the second transducer to deliver pulsed field ablation energy during the delivery of at least part of the first high voltage pulse train set, e.g., in at least some embodiments where it is desired that multiple transducers contemporaneously perform PFA. According to various embodiments, the information indicative of temperature at least proximate the second transducer is provided by the second transducer. For example, the second transducer may include a temperature sensor configured to provide the information indicative of temperature at least proximate the second transducer.

According to various embodiments, temperature may be sensed according to any of multiple techniques (for example, as described above with sensor 408 which employs a resistive element whose resistance varies with temperature). In some embodiments, temperature may be measured continuously over a period of time during which pulses are delivered. Each pulse presents a very rapid delivery of a large amount of energy, but that effect is quickly averaged out so that the overall temperature over a period of time should change fairly smoothly in some embodiments.

In FIG. 6D, according to some embodiments, block 636 represents a configuration of the data processing device system (e.g., data processing device system 110 or 310) to determine, based at least on the information indicative of temperature at least proximate the second transducer, a particular pulse train parameter set of each respective second high voltage pulse train of a second high voltage pulse train set. According to various embodiments, each high voltage pulse train of the second high voltage pulse train set is configured to cause pulsed field ablation of tissue. For instance, with respect to FIG. 7C, a first high voltage pulse train 732c may be delivered and, based on information indicative of a sensed temperature, one or more parameters for a subsequent pulse train may be determined, which may result in the delivery of second high voltage pulse train 734c according to some embodiments.

According to various embodiments, the particular pulse train parameter set of each respective second high voltage pulse train includes at least one pulse train parameter that is different than a corresponding pulse train parameter of a pulse train parameter set of the respective first high voltage pulse train delivered by the first transducer during the first cardiac cycle. For example, in some embodiments, the at least one pulse train parameter of the determined particular pulse train parameter set of each respective second high voltage pulse train includes a particular number of high voltage pulses (e.g., particular number 738c in the example of FIG. 7C) in the respective second high voltage pulse train (e.g., high voltage pulse train 734c). In some embodiments, the at least one pulse train parameter of the determined particular pulse train parameter set of each respective second high voltage pulse train includes a particular pulse amplitude (or voltage) of each of one or more high voltage pulses in the second high voltage pulse train. In some embodiments, the at least one pulse train parameter of the determined particular pulse train parameter set of each respective second high voltage pulse train includes a particular pulse shape of each of one or more high voltage pulses in the second high voltage pulse train. In some embodiments, the at least one pulse train parameter of the determined particular pulse train parameter set of each respective second high voltage pulse train includes a particular inter-pulse spacing (e.g., inter-pulse spacing 746c) between high voltage pulses in the second high voltage pulse train. In some embodiments, reduction in the high voltage pulse rate of a pulse train delivered per heartbeat may be employed to avoid adverse thermal effects as discussed herein.

In FIG. 6D, according to some embodiments, block 638 represents a configuration of the data processing device system (e.g., data processing device system 110 or 310) to cause, via the input-output device system (e.g., 110, 310), each of at least a third transducer of the plurality of transducers, to deliver a respective second high voltage pulse train of the second high voltage pulse train set with the determined particular parameter set during a second cardiac cycle subsequent to the first cardiac cycle. For example, while some examples with respect to FIG. 7C (or others of FIG. 7) are described in the context of first and second pulse trains (e.g., pulse trains 732c, 734c in FIG. 7C) being delivered by one transducer, one or more pulse trains may be delivered by one or more of the same or different transducers according to various embodiments. In this regard, for instance, pulse train 734c in FIG. 7C may be delivered by another transducer (e.g., 220, 306, 406).

In some embodiments, the information indicative of temperature at least proximate the second transducer during, or after, delivery of at least part of the first high voltage pulse train set indicates an increase in temperature. In some embodiments, the determined particular pulse train parameter set of the respective second high voltage pulse train delivered by the third transducer is configured to cause the high voltage pulses of the respective second high voltage pulse train delivered by the third transducer to cumulatively deliver less energy during the second cardiac cycle than energy cumulatively delivered during the first cardiac cycle by the high voltage pulses of the respective first high voltage pulse train delivered by the first transducer. In some embodiments, the third transducer is the first transducer, such that, e.g., the first high voltage pulse train 732c and the second high voltage pulse train 734c are delivered at least by the same transducer in some embodiments of at least FIG. 7C. In some embodiments, the third transducer is other than the first transducer, such that, e.g., the first high voltage pulse train 732c and the second high voltage pulse train 734c are delivered at least by different transducers in some embodiments of at least FIG. 7C. In some embodiments, each of the first transducer, the second transducer, and the third transducer is a pulsed field ablation transducer.

Tissue matter typically includes a distribution of tissues with differing electrical properties. Unlike in metallic conductors, electrical conduction within tissue arises from the movement of ions. Electrical current flow through the tissue is related to the tissue's conductivity which is dependent of the particular ion composition in the tissue and the ability of the ions to move within the tissue. It is noted that tissue ion mobility is also temperature dependent. Tissue matter also includes dielectric properties that give rise to electrical behavior that may change with time. Both conductivity and relative permittivity vary widely between different tissues and these parameters also vary with the frequency of the applied field. Tissue permittivity is related to the extent to which electrical charge within the tissue matter can be displaced or polarized under the influence of one or more electric fields. Measured tissue permittivity may be a function of the sampling frequency.

According to various embodiments, one or more PFA high voltage pulse train parameters may be adjusted over the delivery of therapy in response to the warming of the tissue. In some cases, the conductivity of cardiac tissue increases approximately 2% per degree Celsius increase in temperature of the cardiac tissue, and reducing various PFA high voltage pulse train parameters such as the number of pulses, pulse duration, or waveform, or pulse shape (for example as described above to compensate for the average power delivered) may also serve to mitigate undesired thermal effects even where temperature was not directly measured. To the same end, various PFA high voltage pulse train parameters may be adjusted to vary the power to control the corresponding change in an impedance characteristic (e.g., resistance, reactance) that may arise as a consequence of the temperature rise of the underlying tissue. It is noted that different tissues may experience different changes in conductivity in response to temperature changes.

According to various embodiments, one or more PFA high voltage pulse train parameters may be varied based at least on the particular energy per high voltage pulse (e.g., the energy per high voltage pulse indicated by the voltage, current and duration of the high voltage pulse or one or more recent high voltage pulses). In some cases, application of a predefined voltage may lead to a delivery of a high voltage pulse with more power than required where tissue electrical conductivity is higher than expected. According to various embodiments, it may be desirable in such cases to adjust a pulse train parameter to avoid potentially adverse thermal effects. For example, in some embodiments, reduction in the high voltage pulse rate of a pulse train delivered per heartbeat may be employed in response to the suspected higher tissue conductivity. Other parameters such as the pulse voltage or the pulse duration may be reduced in some embodiments with the use of a possibly more complicated control effort. In some embodiments, one or more PFA pulse train parameters may be varied based at least on information related to an impedance of a PFA high voltage pulse (e.g., a test pulse in some embodiments) or from an earlier delivered PFA high voltage (either during a particular heartbeat or as assessed over one or more previous heartbeats). As used herein, in some embodiments, information related to an impedance may include (a) information related to a resistance, (b) information related to a reactance, or both (a) and (b). Impedance, by definition includes resistive and reactive components (i.e., it is typically expressed as a vector). In various embodiments, the resistive component may be of primary importance when limiting thermal effects, since any purely reactive component may not cause significant power dissipation. Further, in some implementations, only a scalar measurement of the impedance may be available, which does not provide any vector information.

According to various embodiments, the impedance seen by a PFA driver during delivery of a PFA high voltage pulse delivery will be affected by the conductivity of adjacent tissue, which may vary depending on anatomy and composition of the tissue as well as of the composition of other surrounding structures.

FIG. 6E illustrates a programmed configuration 600 of a data processing device system (e.g., 110, 310), according to some embodiments of the present invention. In some embodiments in which the programmed configuration illustrated in FIG. 6E actually is executed at least in part by the data processing device system, such actual execution may be considered a respective method executed by the data processing device system. In this regard, reference numeral 640 and FIG. 6E may be considered to represent one or more methods in some embodiments and, for ease of communication, one or more methods 640 may be referred to at times simply as method 640. The blocks shown in FIG. 6E may be associated with computer-executable instructions of a program that configures the data processing device system to perform the actions described by the respective blocks. According to various embodiments, not all of the actions or blocks shown in FIG. 6E are required, and different orderings of the actions or blocks shown in FIG. 6E may exist. In this regard, in some embodiments, a subset of the blocks shown in FIG. 6E or additional blocks may exist. In some embodiments, a different sequence of various ones of the blocks in FIG. 6E or actions described therein may exist.

In some embodiments, a memory device system (e.g., 130, 330 or a computer-readable medium system) stores the program represented by FIG. 6E, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions provided by the blocks of method 640 via interaction with at least, for example, a transducer-based device (e.g., PFA devices 200A, 300A, or 400A). In these various embodiments, the program may include instructions configured to perform, or cause to be performed, various ones of the block actions described by or otherwise associated with one or more or all of the blocks illustrated in FIG. 6E for performance of some or all of method 640.

FIG. 6E includes block 642, which represents a configuration of the data processing device (e.g., 110, 310) to cause, via the input-output device system (e.g., 120, 320), each of at least a first transducer of a plurality of transducers (e.g., 220, 306, 406) located on a catheter device, to deliver a respective first high voltage pulse train (e.g., high voltage pulse train 732c) of a first high voltage pulse train set during a first cardiac cycle (e.g., cardiac cycle 720c). According to various embodiments, each high voltage pulse train of the first high voltage pulse train set is configured to cause pulsed field ablation of tissue. According to various embodiments, each of the plurality of transducers (e.g., 220, 306, 406) is deliverable by the catheter device to a respective location within a patient (e.g., a respective location in a bodily opening or a respective location in a bodily organ).

FIG. 6E includes block 644, which represents a configuration of the data processing device system (e.g., 110, 310) to cause reception, via the input-output device system (e.g., 120, 320), of particular information, the particular information indicative at least of, or based at least on, impedance (e.g., tissue impedance) at least proximate a second transducer of the plurality of transducers during, or after, delivery of at least part of the first high voltage pulse train set. In some embodiments, the particular information is based at least on the resistance of the load (e.g., the tissue), which is typically indicative of the power generated in the tissue and is of primary concern for limiting thermal effects. It is noted that typical circuits for measuring voltage and current are not located at the load, but are located upstream (for example, in the controller system (e.g., controller system 324)). Accordingly, it may be necessary to adjust the impedance (or resistance) measured by the controller system to account for the upstream circuitry in order to determine the impedance of the tissue. In various embodiments, this adjustment may involve various calibrations to sufficiently account for the electrical characteristics of the controller system, cable, and catheter used to perform the procedure.

In some embodiments, the particular information is provided by the second transducer. In various embodiments, the particular information is based at least on impedance determined at least in part from one or more high voltage pulses configured to cause pulsed field ablation. In some embodiments, the one or more high voltage pulses are delivered by the second transducer. In various embodiments, impedance may be measured utilizing any of multiple known techniques including measurements of voltage and current. In some embodiments, impedance may be measured utilizing radio-frequency (RF) signals applied either before or concurrently with the application of PFA pulses or between PFA pulses. In some embodiments, low (non-ablative) voltage pulses are utilized to measure impedance. In some embodiments in which RF energy or low voltage pulses are applied during the application of PFA pulses, individual RF pulses may be measured using an average of the voltage to area ratio. In other words, when estimating impedance, a measured current may be adjusted for the area of the respective electrode. In some embodiments the pulses are collectively averaged as opposed to individually measured. In various embodiments, the magnitude of the actual PFA pulse voltage and current (e.g., the “average” values of the plateaus of the pulses) are measured, with the measured values employed to calculate an impedance magnitude (e.g., abs(Z)=abs(V)/abs(I), where “abs” denotes magnitude, “Z” denotes impedance, “V” denotes voltage, and “I” denotes current). For example, a first PFA pulse may be delivered, and measurements of 2000 V and 50 amps may be made. From these measurements, an impedance of 40 ohms (assuming no adjustment (e.g., as indicated above) is calculated). In some embodiments, impedance may be measured during each PFA pulse. In some embodiments, impedance may be measured, every heartbeat. It is noted that if the PFA pulse is delivered to more than one PFA transducer pair, it may not be possible to determine the distribution of the resulting power dissipation across the various PFA transducers (e.g., primarily the power dissipation across the tissue associated with the various PFA transducers), but one could make the assumption that it is equally distributed.

In some embodiments, in which PFA high voltage pulses may be applied to multiple PFA transducer (e.g., 220, 306, 406) groups or clusters, the groups or clusters may be selected to each contain transducers (e.g., electrodes) having similar electrical characteristics. In this way a common set of PFA high voltage pulse parameters could be applied to each set that are tailored to the local tissue impedance properties.

In some embodiments, impedance measurement with some other stimulus (e.g., other than a PFA pulsed signal) may be conducted. It is noted, however, that the impedance measured with this other stimulus may not be equal to the impedance presented to an actual PFA pulse (due to frequency dependence of tissue impedance).

In FIG. 6E, according to some embodiments, block 646 represents a configuration of the data processing device system (e.g., data processing device system 110 or 310) to determine, based at least on the impedance-based particular information, a particular pulse train parameter set of each respective second high voltage pulse train of a second high voltage pulse train set. According to various embodiments, each high voltage pulse train of the second high voltage pulse train set is configured to cause pulsed field ablation of tissue. For instance, with respect to FIG. 7C, a first high voltage pulse train 732c may be delivered and, based on the impedance-based particular information, one or more parameters for a subsequent pulse train may be determined, which may result in the delivery of second high voltage pulse train 734c according to some embodiments. It is noted, that in some embodiments, the second high voltage pulse train 734c may be delivered during a cardiac cycled 722c that does not immediately succeed cardiac cycle 720c.

According to various embodiments, the particular pulse train parameter set of each respective second high voltage pulse train includes at least one pulse train parameter that is different than a corresponding pulse train parameter of a pulse train parameter set of the respective first high voltage pulse train delivered by the first transducer during the first cardiac cycle. For example, in some embodiments, the at least one pulse train parameter of the determined particular pulse train parameter set of each respective second high voltage pulse train includes a particular number of high voltage pulses (e.g., particular number 738c in the example of FIG. 7C) in the respective second high voltage pulse train (e.g., high voltage pulse train 734c). In some embodiments, the at least one pulse train parameter of the determined particular pulse train parameter set of each respective second high voltage pulse train includes a particular pulse amplitude (or voltage) of each of one or more high voltage pulses in the second high voltage pulse train. In some embodiments, the at least one pulse train parameter of the determined particular pulse train parameter set of each respective second high voltage pulse train includes a particular pulse shape of each of one or more high voltage pulses in the second high voltage pulse train. In some embodiments, the at least one pulse train parameter of the determined particular pulse train parameter set of each respective second high voltage pulse train includes a particular inter-pulse spacing (e.g., inter-pulse spacing 746c) between high voltage pulses in the second high voltage pulse train. In some embodiments, reduction in the high voltage pulse rate of a pulse train delivered per heartbeat may be employed to avoid adverse thermal effects as discussed herein.

In FIG. 6E, according to some embodiments, block 648 represents a configuration of the data processing device system (e.g., data processing device system 110 or 310) to cause, via the input-output device system (e.g., 110, 310), each of at least a third transducer of the plurality of transducers, to deliver a respective second high voltage pulse train of the second high voltage pulse train set with the determined particular parameter set during a second cardiac cycle subsequent to the first cardiac cycle. For example, while some examples with respect to FIG. 7C (or others of FIG. 7) are described in the context of first and second pulse trains (e.g., pulse trains 732c, 734c in FIG. 7C) being delivered by one transducer, one or more pulse trains may be delivered by one or more of the same or different transducers according to various embodiments. In this regard, for instance, pulse train 734c in FIG. 7C may be delivered by a particular transducer (e.g., 220, 306, 406), which may be the same or different than another transducer that delivers pulse train 732c, according to various embodiments.

In some embodiments, the particular information indicates a decrease in impedance at least proximate the second transducer. In some embodiments, the determined particular pulse train parameter set of the respective second high voltage pulse train delivered by the third transducer is configured to cause the high voltage pulses of the respective second high voltage pulse train delivered by the third transducer to cumulatively deliver less energy during the second cardiac cycle than energy cumulatively delivered during the first cardiac cycle by the high voltage pulses of the respective first high voltage pulse train delivered by the first transducer. In some embodiments, the third transducer is the first transducer, such that, e.g., the first high voltage pulse train 732c and the second high voltage pulse train 734c are delivered at least by the same transducer in some embodiments of at least FIG. 7C. In some embodiments, the third transducer is other than the first transducer, such that, e.g., the first high voltage pulse train 732c and the second high voltage pulse train 734c are delivered at least by different transducers in some embodiments of at least FIG. 7C. In some embodiments, the second transducer is the first transducer. In some embodiments, each of the first transducer, the second transducer, and the third transducer is a pulsed field ablation transducer.

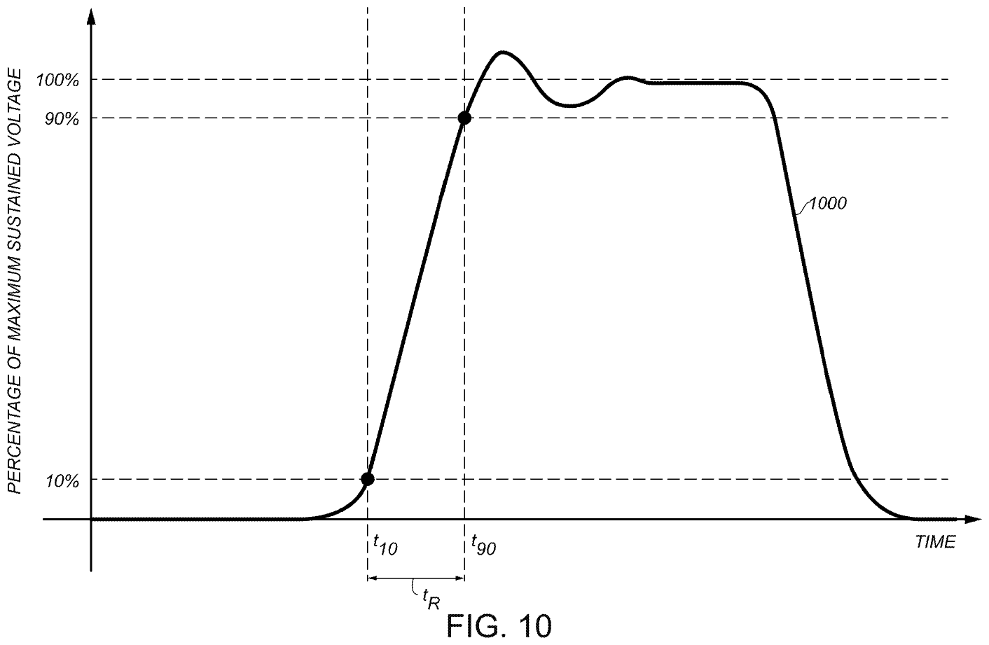

Each high voltage pulse has a rise time and a fall time. In some embodiments, rise time refers to the time it takes for the leading edge of a pulse (e.g., a voltage pulse or current pulse) to rise from a lower threshold value to an upper threshold value. Fall time is the time it takes for the pulse to move from the upper threshold value to the lower threshold value, according to some embodiments. In some embodiments, the upper threshold value is a maximum or peak value (maximum or peak voltage value or maximum or peak current value) of the pulse, and the lower threshold value is the minimum or lowest value (minimum or lowest voltage value or minimum or lowest current value) of the pulse. In some embodiments, the upper threshold value is a first percentage (e.g., selected from within a range of 80% to 100% according to various embodiments) of the maximum or peak value of the pulse, and the lower threshold value is a second percentage (e.g., selected from within a range of 0% to 20% according to various embodiments) of the maximum or peak value of the pulse, the second percentage lower than the first percentage. In some embodiments, the upper threshold value is a first percentage (e.g., selected from within a range of 80% to 100% according to various embodiments) of the maximum sustained value (e.g., a maximum sustained voltage or current) of the pulse, and the lower threshold value is a second percentage (e.g., selected from within a range of 0% to 20% according to various embodiments) of the maximum sustained value of the pulse, the second percentage lower than the first percentage. For example, FIG. 10 shows an example pulse 1000 in which the rise time t_{R} is determined as the time elapsed from the time t₁₀, at which a lower threshold of 10% of maximum sustained voltage is reached, to a time t₉₀, at which an upper threshold of 90% of maximum sustained voltage is reached. Fall time may be determined in a similar manner according to various embodiments. A biphasic pulse may be used for any of the PFA pulses described herein, according to various embodiments. It is noted that each phase of the biphasic pulse has its own rise time and its own fall time, for example, as described herein. It is noted that rise time and fall time may be defined for an electric current pulse in similar manners.

Rise time and fall time may become significant factors in PFA performance as ever decreasing pulse durations are employed. In various embodiments, application of PFA high voltage pulses for a relatively short duration may be desirable in some contexts. For example, the present inventors have found improved performance in lesion formation while reducing undesired muscle contraction effects for a given lesion depth by applying high voltage PFA pulses for a relatively short duration. It is noted that the impact of rise time and fall time (each typically in the realm of about a microsecond or so in some embodiments) may have an adverse impact on the pulse energy deliverable by these short duration pulses. Further, the present inventors have noted that, in systems in which the high voltage pulses are deliverable by different groups of PFA transducers (e.g., electrodes), the rise times of the deliverable pulses will vary in accordance with which particular group of electrodes delivers the pulses. It is noted that a PFA driver circuit may, in some embodiments, be considered to be basically an LR circuit (e.g., an inductor of inductance L connected in series with a resistor of resistance R), for which the time constant is L/R. The present inventors have noted that, when the PFA pulses are delivered to increasing numbers of electrodes (e.g., electrodes each having a same sized energy transmission surface), the resistance provided by the tissue decreases, and the time constant increases, and, therefore, the rise times of the pulses increase. Without taking special steps to mitigate this effect, the energy deliverable by the electrodes may vary in accordance with which particular clusters or groups of the electrodes are selected to have the PFA pulses delivered therebetween. Typically, the highest inter-electrode resistance (shortest rise time) will correspond to only one pair of PFA electrodes and the inter-electrode resistance will typically decrease (while the rise time increases, i.e., gets longer) with larger numbers of PFA electrodes (e.g., as described below in this disclosure). PFA systems that employ transformer-based generators will have some leakage inductance and so rise time will depend on load resistance. The response may also depend on other circuit elements which may be parasitic or which may be intentionally added to modify response. Other PFA systems that employs a switched-capacitor design may not have an associated leakage inductance. However, at some point, rise time would still be limited by inductance of the catheter and catheter cable as well as other parasitic elements.

The following first order approximation may be used to explain the rise time vs. resistance trend. According to various embodiments, a step response may be provided by equation (4):

v=Vₚ(1−e^{-t/τ}) (4)

where:
v is instantaneous voltage;
Vₚ is the peak voltage;
t is time; and
τ is the time constant (with time constant τ=L/R).

According to various embodiments, the rise time (i.e., between 10% and 90%) may be given by equation (5):

$${t}_{r} = τ ln 9 = \frac{L}{R} ln 9 \qquad ( 5 )$$

where tᵣ is the rise time;
L is the inductance; and
R is the resistance.

A shorter rise time (e.g., a relatively fast rise time), t_{r(lower)}≈1 μs, has been calculated by the present inventors for one particular driver configuration using equation (5) and this equation would correspond to the maximum load resistance R_{L}=142Ω (e.g., associated with a pair of PFA electrodes according to some embodiments). At a lower load resistance (e.g., associated with multiple pairs of activated PFA electrodes according to some embodiments) the rise time has been calculated to increase to an upper value tᵣ₍ᵤₚₚₑᵣ₎=3.6 μs (e.g., a relatively slow rise time). With short duration pulses (e.g., pulse widths of under 10 μs in some embodiments, or pulse widths of under 5 μs in some embodiments), the pulse energy delivered per pulse may materially change based on which group of electrodes is activated to deliver the PFA pulses. Even in the absence of rise time/fall time considerations, energy delivered per pulse will change significantly based on load changes (e.g., the number of electrodes changes the resistance across which the voltage is being applied and the energy is inversely proportional to this resistance). It is noted that since typical PFA systems will typically connect to the catheter through a catheter cable having a relatively long length and many closely spaced conductors, relatively high parasitic shunt capacitance may be present. In some embodiments, the rise time may be more closely modelled as a second order system when parasitic capacitance appears in parallel with load resistance. It is noted that pulse fall times will be affected in a similar manner to changes in pulse rise time discussed above or otherwise in this disclosure.

FIG. 6F illustrates a programmed configuration 650 of a data processing device system (e.g., 110, 310), according to some embodiments of the present invention. In some embodiments in which the programmed configuration illustrated in FIG. 6F actually is executed at least in part by the data processing device system, such actual execution may be considered a respective method executed by the data processing device system. In this regard, reference numeral 650 and FIG. 6F may be considered to represent one or more methods in some embodiments and, for ease of communication, one or more methods 650 may be referred to at times simply as method 650. The blocks shown in FIG. 6F may be associated with computer-executable instructions of a program that configures the data processing device system to perform the actions described by the respective blocks. According to various embodiments, not all of the actions or blocks shown in FIG. 6F are required, and different orderings of the actions or blocks shown in FIG. 6F may exist. In this regard, in some embodiments, a subset of the blocks shown in FIG. 6F or additional blocks may exist. In some embodiments, a different sequence of various ones of the blocks in FIG. 6F or actions described therein may exist.

In some embodiments, a memory device system (e.g., 130, 330 or a computer-readable medium system) stores the program represented by FIG. 6F, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions provided by the blocks of method 650 via interaction with at least, for example, a transducer-based device (e.g., PFA devices 200A, 300A, or 400A). In these various embodiments, the program may include instructions configured to perform, or cause to be performed, various ones of the block actions described by or otherwise associated with one or more or all of the blocks illustrated in FIG. 6F for performance of some, or all, of method 650.

FIG. 6F shows configurations of the data processing device system to behave differently in association with different states, respectively referred to by blocks 654a, 654b. In this regard, either or both of the states and corresponding actions set forth in blocks 654a, 654b may actually occur or be executed by the data processing device system (e.g., as in a method) in some embodiments, and, in the case where both states and corresponding actions referred to by blocks 654a, 654b actually occur or are executed by the data processing device system, they may occur in any order, as illustrated by the double-headed broken line arrow shown in FIG. 6F between blocks 654a, 654b, according to various embodiments.

In FIG. 6F, according to some embodiments, block 652 represents a configuration of the data processing device system (e.g., data processing device system 110 or 310) to cause identification of a particular pulsed field ablation transducer (e.g., 220, 306, 406) set (e.g., also known as a particular set of pulsed field ablation transducers in some embodiments) of a catheter device, the particular pulsed field ablation transducer set identified from the plurality of pulsed field ablation transducers of the catheter device. For instance, according to various embodiments, the particular pulsed field ablation transducer set may be identified based at least in part on (a) a user selection, e.g., via a user-interface of input-output device system 120, (b) a machine or computer-based selection, e.g., including an analysis performed by the data processing device system 110 or 310 of sensed information (e.g., temperature, transducer or electrode-to-tissue contact, impedance, according to various embodiments), or both (a) and (b). See, e.g., U.S. Pat. No. 9,439,713, issued Sep. 13, 2016, identifying Reinders et al. as inventors, regarding user-based, machine-based, and automatic selections of transducers, such disclosure of which is hereby incorporated herein by reference. According to various embodiments, the particular pulsed field ablation transducer set is identified to be activated to apply a high voltage pulse train between the pulsed field ablation transducers of the particular pulsed field ablation transducer set, the high voltage pulse train sufficient to cause pulsed field ablation of tissue.

In the context of pulsed field ablation, during delivery of the high voltage pulse train between the pulsed field ablation transducers of the particular pulsed field ablation transducer set, each of a first number of the pulsed field ablation transducers will act as an anode pulsed field ablation transducer and each of a second number of the pulsed field ablation transducers of the particular pulsed field ablation transducer set will act as a cathode pulsed field ablation transducer. In various embodiments, activation of the identified particular pulsed field ablation transducer set includes applying a high voltage pulse train between the pulsed field ablation transducers to cause bipolar pulsed field ablation. In various embodiments, activation of the identified particular pulsed field ablation transducer set includes applying a high voltage pulse train between the pulsed field ablation transducers to cause monopolar pulsed field ablation.

As indicated above in this disclosure, the highest inter-electrode resistance will typically correspond to only one pair of PFA electrodes and will typically decrease with large numbers of PFA electrodes. For example, the first order approximation (e.g., equation (4)) is primarily governed by the load resistance between the anode pulsed field ablation transducers and the cathode pulsed field ablation transducers of the identified particular pulsed field ablation transducer set. Assuming that there are M anode pulsed field ablation transducers (e.g., electrodes) and N cathode pulsed field ablation transducers (e.g., electrodes), the load (e.g., tissue) resistance may be provided by the following equation:

R_{L}=R_{A}+R_{C} (6)

where:

R_{L} is the load (tissue) resistance;

R_{A} is the anode pulsed field ablation transducer (electrode) resistance which can be provided by:

$${R}_{A} = \frac{1}{\frac{1}{{R}_{A 1}} + \frac{1}{{R}_{A 2}} + \frac{1}{{R}_{A 3}} + … + \frac{1}{{R}_{AM}}}$$

R_{C} is the cathode pulsed field ablation transducer (electrode) resistance which can be provided by:

$${R}_{C} = \frac{1}{\frac{1}{{R}_{C 1}} + \frac{1}{{R}_{C 2}} + \frac{1}{{R}_{C 3}} + … + \frac{1}{{R}_{CN}}}$$

If resistance associated with all electrodes were exactly the same (R_{E}=R_{Am}=R_{Cn}) load (7) resistance would be reduced to:

$${R}_{L} = ( \frac{1}{M} + \frac{1}{N} ) {R}_{E} \qquad ( 7 )$$

With this simplification, it can be seen that the load (e.g., tissue) resistance decreases with increasing numbers of (a) the anode pulsed field ablation transducers (electrodes), (b) the cathode pulsed field ablation transducers (e.g., electrodes), or both (a) and (b). It is also noted that the load (e.g., tissue) resistance is dependent on the total number of anode pulsed field ablation transducers and cathode pulsed ablation transducers. It is noted that the rise time discussed above (e.g., equation (5)) would be the same (e.g., under same electrode resistance conditions) when an inequality exists between the number of anode pulsed field ablation transducers (e.g., electrodes) and the number of cathode pulsed field ablation transducers (e.g., electrodes), regardless of whether the inequality indicates a greater number of anode pulsed field ablation transducers (e.g., electrodes) or the inequality indicates a greater number of cathode pulsed field ablation transducers (e.g., electrodes).

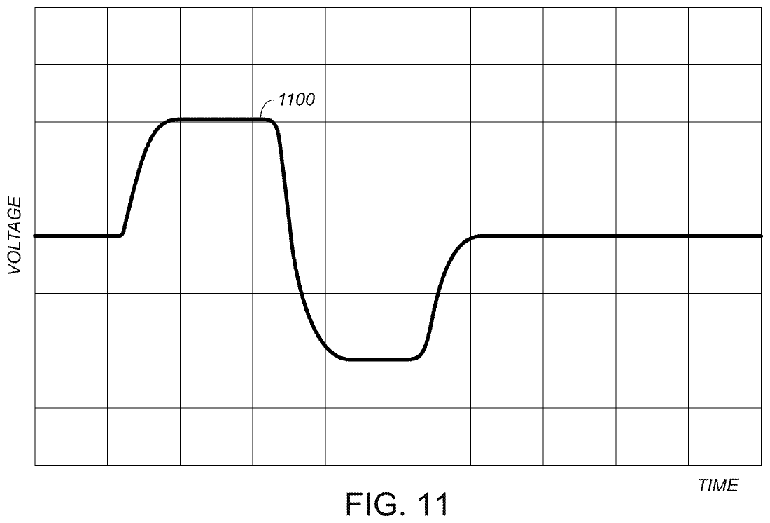

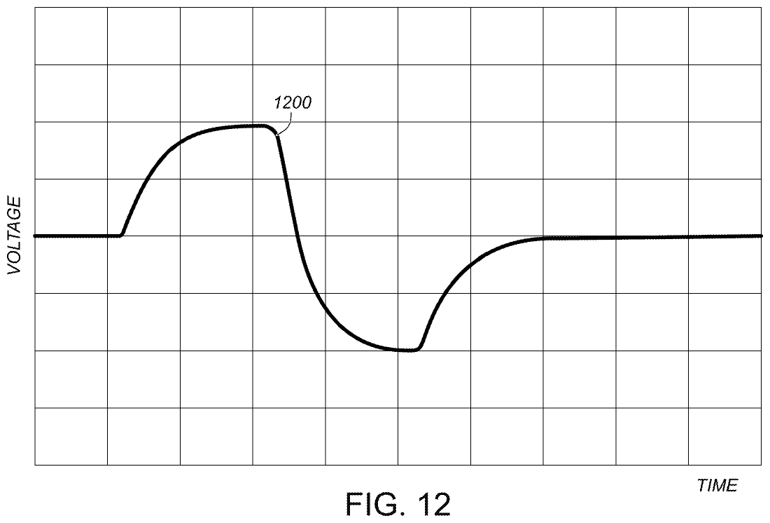

FIG. 11 shows a biphasic voltage pulse waveform 1100 corresponding to a relatively high load (e.g., tissue) resistance of 200Ω, while FIG. 12 shows a biphasic voltage pulse waveform 1200 corresponding to a relatively lower load (e.g., tissue) resistance of 50Ω. The voltage biphasic pulses of FIGS. 11 and 12 were delivered with the same driver voltages and with the same pulse parameters, but with different load resistances. As indicated above, and elsewhere in this disclosure, it is noted that relatively lower resistive loads may be associated with relatively higher numbers of PFA transducers (e.g., electrodes), while relatively higher resistive loads may be associated with relatively lower numbers of PFA transducers (e.g., electrodes), according to various embodiments.

A comparison of the voltage waveforms 1100, 1200 of FIGS. 11 and 12 indicate differences. For example, the biphasic voltage pulse waveform 1100 of FIG. 11 includes relatively short rise times and fall times under the influence of a relatively high load (e.g., tissue) resistance, and the biphasic voltage pulse waveform 1200 of FIG. 12 includes relatively longer rise times and fall times under the influence of a relatively lower load (e.g., tissue) resistance. In various embodiments, the differences in the pulse waveforms are sufficient to change the energy delivered by each of the pulses. The pulse energy delivered per pulse may materially change based on which group of electrodes is activated to deliver the PFA pulses. The pulse train parameter changes as described below and elsewhere in this disclosure may be employed to account for these changes in pulse energy. It is noted that the rise time/fall time of each of the pulses is not solely determined by the patient load resistance, but may also be dependent on various system components (e.g., such as resistors deliberately added in parallel, or nearly in parallel, with the load to reduce overshoot of the voltage waveform, known in some embodiments as “dampers”, “damper resistors”, or “damping resistors”).

In some embodiments, the identified particular pulsed field ablation transducer set is identified based at least on a selection of at least two pulsed field ablation transducers (e.g., 220, 306, 406) of the catheter device, each pulsed field ablation transducer of the at least two pulsed field ablation transducers configured to selectively deliver energy sufficient for pulsed field ablation of tissue. In some embodiments, the selection of the at least two pulsed field ablation transducers (e.g., 220, 306, 406) of the catheter device is a user selection of the at least two pulsed field ablation transducers (e.g., a user selection made via the input-output device system (e.g., 110, 310). In some embodiments, the selection of the at least two pulsed field ablation transducers (e.g., 220, 306, 406) is a machine-based or computer-based selection of the at least two pulsed field ablation transducers. In some embodiments, an initial set of pulsed field ablation transducers (e.g., 220, 306, 406) is identified (e.g., via a user-selection) from the plurality of pulsed field ablation transducers of the catheter device, and the identified particular pulsed field ablation transducer set is identified (e.g., via a machine-based or computer-based selection) as a subset of the initial set of pulsed field ablation transducers, the subset of the initial set of pulsed field ablation transducers numbering fewer than the pulsed field ablation transducers in the initial set of pulsed field ablation transducers. The identification of subsets of the initial set of pulsed field ablation transducers may be motivated for different reasons. For example, in some embodiments, driver limitations may limit the number of pulsed field ablation transducers that may be concurrently activated. Sequential activation of various subsets of the initial set of pulsed field ablation transducers may be implemented to overcome driver limitations, according to some embodiments.

In some embodiments, the identified particular pulsed field ablation transducer set is identified based at least on an analysis of degree of tissue contact exhibited by at least the pulsed field ablation transducers of the particular pulsed field ablation transducer set. For example, it may be advantageous in some embodiments to identify particular pulsed field ablation transducers having the same or similar degree of tissue contact for activation (e.g., concurrent activation) to deliver pulse trains with a particular desired pulse train parameter set (e.g., as described in this disclosure). In some embodiments, the identified particular pulsed field ablation transducer set is identified based at least on an analysis of data provided by each pulsed field ablation transducer of at least the pulsed field ablation transducers of the particular pulsed field ablation transducer set. For example, in some embodiments, the data provided by each pulsed field ablation transducer of at least the pulsed field ablation transducers of the particular pulsed field ablation transducer set may indicate various characteristics, such as degrees of contact, temperature, or impedance by way of non-limiting examples. In some embodiments, particular pulsed field ablation transducers whose data indicates the same or similar characteristics may be identified as a particular grouping forming (at least part of) the identified particular pulsed field ablation transducer set.

In FIG. 6F, according to some embodiments, block 654 represents a configuration of the data processing device system (e.g., data processing device system 110 or 310) (according to a program) to cause activation of the particular pulsed field ablation transducer set (identified per block 652) to deliver a high voltage pulse train. In this regard, such activation may be executed differently dependent on a makeup or characteristics of the identified particular pulsed field ablation transducer set, as shown by the example states of blocks 654a and 654b in FIG. 6F, according to some embodiments. Further in this regard, either or both of the example states illustrated by blocks 654a and 654b may occur, and in the case where both states occur, they may occur in either order, as illustrated by the double-headed broken line arrow in FIG. 6F between blocks 654a and 654b, according to various embodiments.

In some embodiments, block 654a represents a configuration of the data processing device system (e.g., data processing device system 110 or 310) (according to a program) to, in association with a first state in which the identified particular pulsed field ablation transducer set (e.g., identified per block 652) is a first set of pulsed field ablation transducers of the catheter device, determine a first particular parameter set of the high voltage pulse train and cause activation, via the input-output device system (e.g., 120, 320), of the identified particular pulsed field ablation transducer set to deliver the high voltage pulse train in accordance with the determined first particular parameter set. In FIG. 6F, according to some embodiments, block 654b represents a configuration of the data processing device system (e.g., data processing device system 110 or 310) (according to a program) to, in association with a second state in which the identified particular pulsed field ablation transducer set (e.g., identified per block 652) is a second set of pulsed field ablation transducers of the catheter device different than the first set of pulsed field ablation transducers, determine a second particular parameter set of the high voltage pulse train, and cause activation, via the input-output device system (e.g., 120, 320), of the identified particular pulsed field ablation transducer set to deliver the high voltage pulse train in accordance with the determined second particular parameter set. According to various embodiments, the second particular parameter set of the high voltage pulse train is different than the first particular parameter set. In some embodiments, the high voltage pulse train is a first high voltage pulse train of a plurality of high voltage pulse trains. In some embodiments, the data processing device system (e.g., 110, 310) is configured at least by the program at least to cause activation, via the input-output device system (e.g., 120, 320), of the particular pulsed field ablation transducer set to deliver each high voltage pulse train of the plurality of high voltage pulse trains during a respective cardiac cycle of a plurality of cardiac cycles.

The particular pulsed field ablation transducer set may take various forms in each of the first state and the second state, according to various embodiments. In some embodiments, differences may occur between the pulsed field ablation transducers in the particular pulsed field ablation transducer set when in the first state as compared to the second state. For example, in some embodiments, in the first state in which the identified particular pulsed field ablation transducer set is the first set of pulsed field ablation transducers of the catheter device, the first set of pulsed field ablation transducers has a first number of pulsed field ablation transducers (e.g., 220, 306, 406), and, in the second state in which the identified particular pulsed field ablation transducer set is the second set of pulsed field ablation transducers of the catheter device, the second set of pulsed field ablation transducers has a second number of pulsed field ablation transducers (e.g., 220, 306, 406) greater than the first number of pulsed field ablation transducers. As discussed in this disclosure, such different numbers of transducers may lead to differences in available conductive surface areas between the first and second sets of pulsed field ablation transducers, thereby causing differences in resistance or impedance, which may be addressed or compensated for by the data processing device system (e.g., 110, 310) by application of different high voltage pulse or pulse train parameter sets for the different states/numbers of transducers. In some embodiments, each pulsed field ablation transducer of the identified particular pulsed field ablation transducer set includes a respective electrode (e.g., 315, 415), each respective electrode including a respective energy delivery surface, energy transmission surface, or conductive surface (e.g., 319) configured to deliver pulsed field ablation energy.

According to various embodiments, in the first state in which the identified particular pulsed field ablation transducer set is the first set of pulsed field ablation transducers of the catheter device, the energy delivery surfaces (which may also be referred to as energy transmission surfaces or conductive surfaces according to various embodiments) of the first set of pulsed field ablation transducers have a first collective area, and, in the second state in which the identified particular pulsed field ablation transducer set is the second set of pulsed field ablation transducers of the catheter device, the energy delivery surfaces (which may also be referred to as energy transmission surfaces or conductive surfaces according to various embodiments) of the second set of pulsed field ablation transducers have a second collective area greater than the first collective area. For example, in at least some cases in which the second set of pulsed field ablation transducers has more transducers than the first set of pulsed field ablation transducers, the second set of pulsed field ablation transducers have a second collective area greater than the first collective area. Even in some cases in which the first and second sets of pulsed field ablation transducers have a same number of transducers, differences in collective conductive surface area may exist, e.g., due to differences in electrode sizes or shapes as shown, e.g., in FIG. 3B, and these differences in collective conductive surface area may be addressed or compensated for by the data processing device system (e.g., 110, 310) by application of different high voltage pulse or pulse train parameter sets for the different states/collective energy delivery surface areas.

In some embodiments, in the first state in which the identified particular pulsed field ablation transducer set is the first set of pulsed field ablation transducers of the catheter device, the energy delivery surfaces of the first set of pulsed field ablation transducers have a first set of one or more geometric shapes, and, in the second state in which the identified particular pulsed field ablation transducer set is the second set of pulsed field ablation transducers of the catheter device, the energy delivery surfaces of the second set of pulsed field ablation transducers have a second set of one or more geometric shapes different than the first set of one or more geometric shapes. For example, the catheter shown in FIG. 3B includes multiple electrodes 315 having energy delivery surfaces having different geometric shapes, with various ones of the multiple electrodes being identifiable for inclusion in the particular pulsed field ablation transducer set. In some embodiments, in the first state in which the identified particular pulsed field ablation transducer set is the first set of pulsed field ablation transducers of the catheter device, each energy delivery surface of at least one energy delivery surface of the first set of pulsed field ablation transducers has a first area, and, in the second state in which the identified particular pulsed field ablation transducer set is the second set of pulsed field ablation transducers of the catheter device, each energy delivery surface of at least one energy delivery surface of the second set of pulsed field ablation transducers has a second area different than the first area (e.g., as shown in FIG. 3B where different transducers have different energy delivery surface areas).

In some embodiments, each energy delivery surface of the first set of pulsed field ablation transducers in the first state has a different area than each energy delivery surface of the second set of pulsed field ablation transducers in the second state. For example, in a first state, a first set of the transducers shown, e.g., in FIG. 3B may be selected such that the selected transducers in the first set each have a different energy delivery surface area than each transducer selected in a second state in which a second set of transducers is selected. Similarly, in some embodiments, in the first state in which the identified particular pulsed field ablation transducer set is the first set of pulsed field ablation transducers of the catheter device, each energy delivery surface of at least one energy delivery surface of the first set of pulsed field ablation transducers has a first geometric shape, and, in the second state in which the identified particular pulsed field ablation transducer set is the second set of pulsed field ablation transducers of the catheter device, each energy delivery surface of at least one energy delivery surface of the second set of pulsed field ablation transducers has a second geometric shape different than the first geometric shape.

In some embodiments, similarities between various ones of the pulsed field ablations transducers in the identified particular pulsed field ablation transducer set occur in each of the first and the second state. In some embodiments, each pulsed field ablation transducer of the identified particular pulsed field ablation transducer set includes a respective electrode (e.g., 315, 415), each respective electrode including a respective energy delivery surface (e.g., bounded by edge 415-1) configured to deliver pulsed field ablation energy. In some embodiments, in the first state in which the identified particular pulsed field ablation transducer set is the first set of pulsed field ablation transducers of the catheter device, the energy delivery surface of each of at least one pulsed field ablation transducer of the first set of pulsed field ablation transducers has a first area, and, in the second state in which the identified particular pulsed field ablation transducer set is the second set of pulsed field ablation transducers of the catheter device, the energy delivery surfaces of each of at least one pulsed field ablation transducer of the second set of pulsed field ablation transducers has a second area the same as the first area. For example, cases may exist where transducers shown, e.g., in FIG. 3B, are included in each of the first and second sets of pulsed field ablation transducers that have a same energy delivery surface area.

Similarly, in some embodiments, (a) in the first state in which the identified particular pulsed field ablation transducer set is the first set of pulsed field ablation transducers of the catheter device, the energy delivery surfaces of the first set of pulsed field ablation transducers have a same area, or (b) in the second state in which the identified particular pulsed field ablation transducer set is the second set of transducers of the catheter device, the energy delivery surfaces of the second set of transducers have a same area. In some embodiments, (c) in the first state in which the identified particular pulsed field ablation transducer set is the first set of pulsed field ablation transducers of the catheter device, the energy delivery surfaces of the first set of pulsed field ablation transducers have a same geometric shape, or (d) in the second state in which the identified particular pulsed field ablation transducer set is the second set of pulsed field ablation transducers of the catheter device, the energy delivery surfaces of the second set of pulsed field ablation transducers have a same geometric shape. In some embodiments, the respective energy delivery surfaces of the first set of pulsed field ablation transducers in the first state have a same area. In some embodiments, the respective energy delivery surfaces of the second set of transducers in the second state have a same area.

Determination of the first particular parameter set of the high voltage pulse train in accordance with block 654a and determination of the second particular parameter set of the high voltage pulse train in accordance with block 654b may be performed in various manners according to various embodiments. For example, in some embodiments, at least one preliminary or test signal may be delivered between the pulsed field ablation transducers of the identified particular pulsed field ablation transducer set in a state in which at least the identified particular pulsed field ablation transducer set is in proximity with tissue. In some embodiments, the at least one preliminary or test signal may have a pulsed waveform. In some embodiments, at least one preliminary or test pulse is delivered between the pulsed field ablation transducers of the identified particular pulsed field ablation transducer set in a state in which at least the identified particular pulsed field ablation transducer set is in proximity with tissue. In some embodiments, the determination of the first particular parameter set in accordance with block 654a includes a delivery of a first preliminary or test signal set between the pulsed field ablation transducers in the first set of pulsed field ablation transducers, and the determination of the second particular parameter set in accordance with block 654b includes a delivery of a second preliminary or test signal set between the pulsed field ablation transducers in the first set of pulsed field ablation transducers.

In some embodiments, tissue resistance is determined by the data processing device system (e.g., 110, 310) in response to the delivery of the at least one preliminary or test pulse. In some embodiments, the at least one preliminary or test pulse is configured to cause tissue ablation. In some embodiments, the at least one preliminary or test pulse is configured to cause irreversible electroporation. In some embodiments, the at least one preliminary or test pulse is configured to cause reversible electroporation. In some embodiments, the at least one preliminary or test pulse is insufficient to cause either reversible electroporation or irreversible electroporation. It is noted that tissue impedance may vary with signal frequency. In some embodiments, employing a preliminary or test signal with characteristics similar to, or the same as, the subsequent application of a PFA signal may provide more accurate results.

Based at least on the determined resistance, the rise time, fall time, or both the rise time and fall time of the at least one preliminary or test pulse can be determined. For example, in some embodiments, a first order approximation (e.g., as provided by equation (5)) or a second order approximation may be employed to estimate or predict the rise time, or fall time, or both the rise time and the fall time. In some embodiments, the data processing device system (e.g., 110, 310) determines the rise time, the fall time, or both the rise time and the fall time from data indicating the shape or profile of the at least one preliminary or test pulse in response to a state in which the at least one preliminary or test pulse is being delivered to tissue.

In some embodiments, the data processing device system (e.g., 110 or 310) (according to a program) is configured to cause a determination of a particular parameter set of the high voltage pulse train (e.g., which may be delivered subsequent to the delivery of the at least one preliminary or test pulse) in response to delivery of the at least one preliminary or test pulse to tissue via the particular pulsed field ablation transducer set. For example, in some embodiments, the data processing device system (e.g., 110, 310) may determine that a subsequent delivery of the high voltage pulse train with pulse characteristics the same, or similar to, those of the at least one preliminary or test pulse may lead to over-current conditions, or may run afoul of other driver limitations. In some embodiments, the data processing device system (e.g., 110, 310) is configured (according to a program) to determine a particular parameter set of the high voltage pulse train to overcome these adverse conditions.

In some embodiments, the data processing device system (e.g., 110 or 310) (according to a program) is configured to cause a determination of a particular parameter set of the high voltage pulse train based at least on the determined rise time, the determined fall time, or both the determined rise time and fall time. For example, in some embodiments, the data processing device system (e.g., 110 or 310) (according to a program) is configured to determine a pulse energy deliverable by the at least one preliminary or test pulse as configured with a particular pulse shape influenced by the determined rise time, the determined fall time, or both the determined rise time and fall time. In some embodiments, the data processing device system (e.g., 110, 310) is configured (according to a program) to determine a particular parameter set of the high voltage pulse train to achieve a same, or substantially a same, average power (for example as described above or elsewhere in this disclosure), in order to compensate for rise time/fall time induced variances in the waveform associated with delivery of the high voltage pulse train via the identified particular pulsed field ablation transducer set. For example, in some embodiments, the data processing device system (e.g., 110, 310) is configured at least by the program at least to cause the high voltage pulse train to deliver, in the first state, a first average power in accordance with the first particular parameter set, and cause the high voltage pulse train to deliver, in the second state, a second average power in accordance with the second particular parameter set. In some embodiments, the second average power is within 10% of the first average power. In this regard, different particular pulsed field ablation transducers sets may employ different pulse train parameter sets when they are employed to deliver the high voltage pulse train. Accordingly, in some embodiments, the data processing device system (e.g., 110, 310) may be configured (according to a program) to, in association with the first state in which the identified particular pulsed field ablation transducer set is the first set of pulsed field ablation transducers of the catheter device, determine the first particular parameter set of the high voltage pulse train and cause activation, via the input-output device system (e.g., 120, 320), of the particular pulsed field ablation transducer set to deliver the high voltage pulse train in accordance with the determined first particular parameter set, while in association with second state in which the identified particular pulsed field ablation transducer set is the second set of pulsed field ablation transducers of the catheter device, determine the second particular parameter set of the high voltage pulse train and cause activation, via the input-output device system (e.g., 120, 320), of the identified particular pulsed field ablation transducer set to deliver the high voltage pulse train in accordance with the determined second particular parameter set. For example, FIG. 7A may represent the first state which results in delivery of the high voltage pulse train 732a according to the determined first particular parameter set by the particular pulsed field ablation transducer set (i.e., the first set of pulsed field ablation transducers in this first state), and FIG. 7B may represent the second state which results in delivery of the high voltage pulse train 734b according to the determined second particular parameter set by the particular pulsed field ablation transducer set (i.e., the second set of pulsed field ablation transducers in this second state).

In some embodiments, in the first state in which the identified particular pulsed field ablation transducer set is the first set of pulsed field ablation transducers of the catheter device, the data processing device system (e.g., 110, 310) may be configured (according to a program) to cause at least a first preliminary or test pulse to be delivered to the first set of pulsed field ablation transducers. In some embodiments, in the second state in which the identified particular pulsed field ablation transducer set is the second set of pulsed field ablation transducers of the catheter device, the data processing device system (e.g., 110, 310) may be configured (according to a program) to cause at least a second preliminary or test pulse to be delivered to the second set of pulsed field ablation transducers. In some embodiments the at least the first preliminary test pulse and the at least the second preliminary test pulse have similar characteristics (e.g., identical in form in some embodiments). In some embodiments, the at least the first preliminary test pulse and the at least the second preliminary test pulse have different characteristics. The at least the first preliminary test pulse and the at least the second preliminary test pulse may be configured or employed in a manner similar to, or the same as, the at least one preliminary or test pulse described above or elsewhere in this disclosure. In some embodiments, determination of the first particular parameter set of the high voltage pulse train in accordance with block 654a and determination of the second particular parameter set of the high voltage pulse train in accordance with block 654b may be based on particular characteristics of the identified particular pulsed field ablation transducer set. In some embodiments, the particular characteristics of the identified particular pulsed field ablation transducer set may be analyzed in addition to an analysis of the at least one preliminary or test pulse described above. For example, one could assume that the load (e.g., tissue) resistance is inversely proportional to the cumulative surface area of the electrodes, and thus it might not be necessary to use a test pulse to approximate the resistance (and therefore the rise time) in some embodiments.

In some embodiments, the particular characteristics of the identified particular pulsed field ablation transducer set may be analyzed in lieu of performing the at least one preliminary or test pulse described above. For example, in some embodiments, the data processing device system (e.g., 110, 310) may be configured (according to a program) to determine, in association with the first state in which the particular pulsed field ablation transducer set is the first set of pulsed field ablation transducers of the catheter device, the first particular parameter set of the high voltage pulse train based upon particular characteristics or composition of the first set of pulsed field ablation transducers, and in association with the second state in which the particular pulsed field ablation transducer set is the second set of pulsed field ablation transducers of the catheter device, determine the second particular parameter set of the high voltage pulse train based upon particular characteristics or composition of the second set of pulsed field ablation transducers. In some embodiments, the determined characteristics or composition of the identified particular pulsed field ablation transducer set may be correlated to an expected or predicted interaction of a given PFA signal with tissue when delivered between the pulsed field ablation transducers of the identified particular pulsed field ablation transducer set. In some embodiments, the expected or predicted interaction may include an expected impedance or resistance generated in response to the delivery of the given PFA signal when delivered between the pulsed field ablation transducers of the identified particular pulsed field ablation transducer set. In some embodiments, the expected impedance or resistance may be an expected tissue impedance or an expected tissue resistance. In some embodiments, the expected impedance or resistance may be an expected overall impedance or resistance that takes in account expected impedances or resistances (e.g., tissue impedances, catheter impedance, cable impedances, etc.) as described above in this disclosure. Different characteristics or compositions including, but not limited to the number of the transducers/electrodes, the resistance of the electrodes, the sizes and dimensions of the electrodes, and the shapes of the electrodes in the identified particular pulsed field ablation transducer set may be considered, according to various embodiments.

In some embodiments, the data processing device system (e.g., 110, 310) is configured at least by the program at least to perform an analysis of a total number of at least the pulsed field ablation transducers of the particular pulsed field ablation transducer set (e.g., when the at least the pulsed field ablation transducers have common characteristics such as same, or substantially same, electrode size). In some embodiments, in the first state, the analysis of the total number of the at least the pulsed field ablation transducers in the particular pulsed field ablation transducer set is an analysis of a total number of pulsed field ablation transducers in the first set of pulsed field ablation transducers, and, in the second state, the analysis of the total number of the at least the pulsed field ablation transducers in the particular pulsed field ablation transducer set is an analysis of a total number of pulsed field ablation transducers in the second set of pulsed field ablation transducers. According some embodiments, in the first state, the first particular parameter set of the high voltage pulse train is determined based at least on the analysis of the total number of pulsed field ablation transducers in the first set of pulsed field ablation transducers, and, in the second state, the second particular parameter set of the high voltage pulse train is determined based at least on the analysis of the total number of pulsed field ablation transducers in the second set of pulsed field ablation transducers. For example, as described above, and elsewhere in this disclosure, according to some embodiments, a determination of the total number of at least the pulsed field ablation transducers of the particular pulsed field ablation transducer set (e.g., when the pulsed field ablation transducers have a same characteristic such as a same or substantially same, electrode size) may be employed to determine a rise time or fall time of a given PFA pulse if delivered via the particular pulsed field ablation transducer set. Determination of the rise time or fall time (e.g., measured, calculated, estimated, or correlated to the total number of the at least the pulsed field ablation transducers of the particular pulsed field ablation transducer set) may, in some embodiments, be employed to determine a particular parameter set of the high voltage pulse train deliverable between the identified particular pulsed field ablation transducer set. In some embodiments, determination, based at least on the analysis, of the particular parameter set of the high voltage pulse train may be made to achieve a same average power (for example as described above or elsewhere in this disclosure), in order to compensate for anticipated rise-time/fall time effects in the waveform associated with delivery of the high voltage pulse train via the identified particular pulsed field ablation transducer set.

In some embodiments, the data processing device system (e.g., 110, 310) is configured at least by the program at least to perform an analysis of a transducer type of each pulsed field ablation transducer of at least the pulsed field ablation transducers of the particular pulsed field ablation transducer set. In some embodiments, in the first state, the analysis of a transducer type of each pulsed field ablation transducer in the at least the pulsed field ablation transducers in the particular pulsed field ablation transducer set is an analysis of a transducer type of each pulsed field ablation transducer in the first set of pulsed field ablation transducers, and, in the second state, the analysis of a transducer type of each pulsed field ablation transducer in the at least the pulsed field ablation transducers in the particular pulsed field ablation transducer set is an analysis of a transducer type of each pulsed field ablation transducer in the second set of pulsed field ablation transducers. According to some embodiments, in the first state, the first particular parameter set of the high voltage pulse train is determined based at least on the analysis of a transducer type of each pulsed field ablation transducer in the first set of pulsed field ablation transducers, and, in the second state, the second particular parameter set of the high voltage pulse train is determined based at least on the analysis of a transducer type of each pulsed field ablation transducer in the second set of pulsed field ablation transducers. For example, as indicated above and elsewhere in this disclosure, determination of the rise time or fall time (e.g., measured, calculated, estimated, or correlated to the total number of the at least the pulsed field ablation transducers of the particular pulsed field ablation transducer set) may, in some embodiments, be dependent on the resistance of various portions (e.g., electrodes) of each pulsed field ablation transducers. In some embodiments, PFA transducers having different transducer types (e.g., electrodes of different size or shape (for example as shown in FIG. 3B), or electrodes made of different materials having different electrical properties) are employed. When transducer type differences exist among the pulsed field ablation transducers of the identified pulsed field ablation transducer set, the electrical resistance provided by the pulsed field ablation transducers of the identified pulsed field ablation transducer set may be dependent on more than the total number of the pulsed field ablation transducers in the identified pulsed field ablation transducer set.

In some embodiments, the data processing device system (e.g., 110, 310) is configured at least by the program at least to perform an analysis of size, shape, or size and shape of each pulsed field ablation transducer of at least the pulsed field ablation transducers of the particular pulsed field ablation transducer set. In some embodiments, in the first state, the analysis of size, shape, or size and shape of each pulsed field ablation transducer in the at least the pulsed field ablation transducers in the particular pulsed field ablation transducer set is an analysis of size, shape, or size and shape of each pulsed field ablation transducer in the first set of pulsed field ablation transducers, and, in the second state, the analysis of size, shape, or size and shape of each pulsed field ablation transducer in the at least the pulsed field ablation transducers in the particular pulsed field ablation transducer set is an analysis of size, shape, or size and shape of each pulsed field ablation transducer in the second set of pulsed field ablation transducers. According to some embodiments, in the first state, the first particular parameter set of the high voltage pulse train is determined based at least on the analysis of size, shape, or size and shape of each pulsed field ablation transducer in the first set of pulsed field ablation transducers, and in the second state, the second particular parameter set of the high voltage pulse train is determined based at least on the analysis of size, shape, or size and shape of each pulsed field ablation transducer in the second set of pulsed field ablation transducers. According to various embodiments, determining a particular parameter set (e.g., the first particular parameter set or the second particular parameter set) based at least on one or more of the various analysis described above may be used to determine the total resistance provided by various portions (e.g., electrodes) of the pulsed field ablation transducers of the identified pulsed field ablation transducer set. In some embodiments, determining a particular parameter set (e.g., the first particular parameter set or the second particular parameter set) based at least on one or more of the various analysis described above may be made to achieve a same, or substantially same, average power (for example as described above or elsewhere in this disclosure), in order to compensate for anticipated rise-time/fall time effects in the waveform associated with delivery of the high voltage pulse train via the identified particular pulsed field ablation transducer set.

Various differences may occur in the high voltage pulse train if the high voltage pulse train is configured in accordance with the first particular parameters set in the first state as compared to if the high voltage pulse train is configured in accordance with the second particular parameter set in the second state. For example, in some embodiments, each high voltage pulse in the high voltage pulse train includes a respective rise time, and the respective rise time of each high voltage pulse of the high voltage pulse train in accordance with the second particular parameter set is (e.g., configured by the data processing device system in accordance with one or more respective pulse parameters to be) longer than the respective rise time of each high voltage pulse of the high voltage pulse train in accordance with the first particular parameter set (e.g., to compensate for impacts on rise time due to corresponding resistance of the respective transducer set). For example, in some embodiments, fixed resistances (e.g., damping resistors) may be added in parallel with the load resistance (ignoring the fact that the cable and catheter is in between the controller and the load) in order to ensure a desired pulse waveform shape (e.g., a pulse waveform with a desired rise time and fall time) is obtained. Depending on the load resistance (e.g., which may vary with the number of activated electrodes), a different degree of damping resistance may be required to obtain a desired waveform shape. The present inventors have employed systems, in some embodiments, that allow for three values: open circuit, 136 ohms, or 68 ohms. In some embodiments, the use of the largest resistance possible for a given load is employed, as this wastes less output power. According to various embodiments, a pre-ablation system test involves determining the appropriate damping resistance for each group of electrodes by applying lower voltage pulses and observing the resulting waveforms. The use of damping resistances may be employed to adjust the rise time/fall times of the PFA pulses to be approximately the same for different transducer sets.

In some embodiments, each high voltage pulse in the high voltage pulse train is configured to deliver a respective amount of pulse energy, and the pulse energy deliverable by each of at least one high voltage pulse in the high voltage pulse train in accordance with the second particular parameter set is (e.g., configured by the data processing device system in accordance with one or more respective pulse parameters to be) less than the pulse energy deliverable by each of at least one high voltage pulse in the high voltage pulse train in accordance with the first particular parameter set (e.g., to compensate for impacts on energy delivery due to corresponding resistance (e.g., tissue resistance) associated with the activation of the respective transducer set). In some embodiments, each of the first particular parameter set and the second particular parameter set defines a respective pulse duration of each of at least one high voltage pulse in the high voltage pulse train, and the respective pulse duration of each of the at least one high voltage pulse in the high voltage pulse train defined in accordance with the second particular parameter set is (e.g., configured by the data processing device system in accordance with one or more respective pulse parameters to be) less than the respective pulse duration of each of the at least one high voltage pulse in the high voltage pulse train defined in accordance with the first particular parameter set (e.g., to compensate for impacts on pulse duration due to corresponding resistance (e.g., tissue resistance) associated with the activation of the respective transducer set). In some embodiments, each of the first particular parameter set and the second particular parameter set defines a respective pulse frequency of the pulses in the high voltage pulse train, and the respective pulse frequency of the pulses in the high voltage pulse train defined in accordance with the second particular parameter set is (e.g., configured by the data processing device system in accordance with one or more respective pulse parameters to be) lower than the respective pulse frequency of the pulses in the high voltage pulse train defined in accordance with the first particular parameter set (e.g., to compensate for impacts on pulse frequency due to corresponding resistance (e.g., tissue resistance) associated with the activation of the respective transducer set). In some embodiments, each of the first particular parameter set and the second particular parameter set defines a respective number of pulses in the high voltage pulse train, and the respective number of pulses in the high voltage pulse train defined in accordance with the second particular parameter set is (e.g., configured by the data processing device system in accordance with one or more respective pulse parameters to be) less than the respective number of pulses in the high voltage pulse train defined in accordance with the first particular parameter set (e.g., to manage proper energy delivery (e.g., average energy delivery) due to corresponding resistance (e.g., tissue resistance) associated with the of the respective transducer set). Differences between the first parameter set and the second parameter set may arise for various reasons, including those described above in this disclosure.

FIG. 6G illustrates a programmed configuration 660 of a data processing device system (e.g., 110, 310), according to some embodiments of the present invention. In some embodiments in which the programmed configuration illustrated in FIG. 6G is executed at least in part by the data processing device system, such actual execution may be considered a respective method executed by the data processing device system. In this regard, reference numeral 660 and FIG. 6G may be considered to represent one or more methods in some embodiments and, for ease of communication, one or more methods 660 may be referred to at times simply as method 660. The blocks shown in FIG. 6G may be associated with computer-executable instructions of a program that configures the data processing device system to perform the actions described by the respective blocks. According to various embodiments, not all of the actions or blocks shown in FIG. 6G are required, and different orderings of the actions or blocks shown in FIG. 6G may exist. In this regard, in some embodiments, a subset of the blocks shown in FIG. 6G or additional blocks may exist. In some embodiments, a different sequence of various ones of the blocks in FIG. 6G or actions described therein may exist.

In some embodiments, a memory device system (e.g., 130, 330 or a computer-readable medium system) stores the program represented by FIG. 6G, and, in some embodiments, the memory device system is communicatively connected to the data processing device system as a configuration thereof. In this regard, in various example embodiments, a memory device system (e.g., memory device systems 130, 330) is communicatively connected to a data processing device system (e.g., data processing device systems 110 or 310) and stores a program executable by the data processing device system to cause the data processing device system to execute various actions provided by the blocks of method 660 via interaction with at least, for example, a transducer-based device (e.g., PFA devices 200A, 300A, or 400A). In these various embodiments, the program may include instructions configured to perform, or cause to be performed, various ones of the block actions described by or otherwise associated with one or more or all of the blocks illustrated in FIG. 6G for performance of some or all of method 660.

FIG. 6G shows configurations of the data processing device system to behave differently in association with different states, respectively referred to by blocks 664a, 664b. In this regard, either or both of the states and corresponding actions set forth in blocks 664a, 664b may actually occur or be executed by the data processing device system (e.g., as in a method) in some embodiments, and, in the case where both states and corresponding actions referred to by blocks 664a, 664b actually occur or are executed by the data processing device system, they may occur in any order, as illustrated by the double-headed broken line arrow shown in FIG. 6G between blocks 664a, 664b, according to various embodiments.

In FIG. 6G, according to some embodiments, block 662 represents a configuration of the data processing device system (e.g., data processing device system 110 or 310) to cause detection, via the input-output device system, of a degree of tissue contact exhibited by a portion of a catheter device. There are many ways to determine tissue contact by the portion of the catheter device. For example, in some embodiments, determining a degree of tissue contact exhibited by the portion of the catheter device may include differentiating non-fluid tissue (e.g., cardiac tissue) from a fluid (e.g., a fluidic tissue such as blood). Four approaches may include by way of non-limiting example, and, depending upon the particular approach(es) chosen, the configuration of various employed transducers may be implemented accordingly:

1. The use of convective cooling of heated transducer elements (e.g., temperature sensors 408) by fluid. A slightly heated transducer element predominately in proximity to bodily fluids will be cooled more readily than when predominately in proximity to tissue (e.g., tissue defining the interior surface(s) of a bodily cavity).

2. The use of tissue impedance measurements. A set of transducers responsive to electrical tissue impedance may be employed in some embodiments. Typically, heart tissue will have higher associated tissue impedance values than the impedance values associated with blood.

3. The use of a difference in dielectric constant (e.g., as a function of frequency) between blood and tissue.

4. The use of transducers that sense force (i.e., force sensors). A set of force detection transducers can be used to determine a degree of contact with a tissue surface.

See also, e.g., U.S. Pat. No. 8,906,011, issued Dec. 9, 2014, identifying inventors Gelbart et al., regarding approaches to differentiate non-blood tissue from liquid such as blood, such disclosure of which is hereby incorporated herein by reference.

Various ones of the above approaches may be used, at least in part, to determine proximity of a transducer to non-fluidic tissue or to fluidic tissue in some embodiments. Various ones of the above approaches may be used, at least in part, to determine contact between a transducer and non-fluidic tissue or contact between a transducer and fluidic tissue in some embodiments. Various ones of the above approaches may be used, at least in part, to determine an amount of an electrically conductive surface portion of an electrode that contacts non-fluidic tissue or contacts fluidic tissue in some embodiments. Various ones of the above approaches may be used, at least in part, to determine an amount of an electrically conductive surface portion of an electrode that is available to contact non-fluidic tissue or available to contact fluidic tissue in some embodiments.

In FIG. 6G, according to some embodiments, block 664 represents a configuration of the data processing device system (e.g., 110 or 310) to cause activation, via the input-output device system (e.g., 120, 320), of a particular pulsed field ablation transducer set to deliver a high voltage pulse train, the high voltage pulse train sufficient to cause pulsed field ablation of tissue. According to various embodiments, block 664a represents a configuration of the data processing device system (e.g., 110 or 310), in response to a first state in which the detected degree of tissue contact (e.g., determined in block 662) is a first degree, to determine a first particular parameter set of at least the high voltage pulse train and cause the activation, via the input-output device system (e.g., 120, 320) of the particular pulsed field ablation transducer set to deliver the high voltage pulse train in accordance with the determined first particular parameter set. In some embodiments, the activation, via the input-output device system (e.g., 120, 320) of the particular pulsed field ablation transducer set to deliver the high voltage pulse train in accordance with the determined first particular parameter set, occurs at least in part during the first state in which the detected degree of tissue contact (e.g., determined in block 662) is the first degree. According to various embodiments, block 664b represents a configuration of the data processing device system (e.g., 110 or 310), in response to a second state in which the detected degree of tissue contact is a second degree, to determine a second particular parameter set of at least the high voltage pulse train different than the first particular parameter set and cause the activation, via the input-output device system, of the particular pulsed field ablation transducer set to deliver the high voltage pulse train in accordance with the determined second particular parameter set. In some embodiments, the activation, via the input-output device system (e.g., 120, 320) of the particular pulsed field ablation transducer set to deliver the high voltage pulse train in accordance with the determined second particular parameter set occurs at least in part during the second state in which the detected degree of tissue contact (e.g., determined in block 662) is the second degree. In some embodiments, the first degree indicates lesser tissue contact than the second degree.

In this regard, different degrees of tissue contact may influence characteristics of the energy delivered to target tissue and non-target tissue or fluid (e.g., blood) and, for at least this reason, some embodiments of the present invention control one or more pulse parameters based on a detected degree of tissue contact. In various embodiments, high voltage pulse rates in various PFA cardiac procedures may be increased for conditions indicating lesser degrees (e.g., low or little) of PFA transducer (e.g., electrode)-to-tissue contact without risking thermal damage to the blood or tissue. In some embodiments, the lesser degrees of transducer-to-tissue contact may indicate, or be associated with, the corresponding electrode having a layer of flowing blood between its surface and the target tissue. When the electrode is in contact with tissue, any Joule heating arising as a consequence of the delivery of PFA pulses is primarily dissipated by conduction either into the tissue or to the overlying blood, where it is removed or dissipated convectively. Joule heating is typically strongest at regions closest to the PFA electrode (e.g., regions closest to electrode edges or where various electrode edges converge sharply or acutely). The Joule heating arising as a consequence of the delivery of PFA pulses typically diminishes rapidly with distance in a way that may be dependent on the details of the electrode geometry.

This principle may be illustrated with an example of a spherical, monopolar electrode in a very large tissue mass. Electric current flux under steady state conditions for this geometry may be modelled to follow an inverse square law with respect to radial distance from the electrode, and the rate of Joule heating is proportional to the square of the current flux. Consequently, the Joule heating decreases by the fourth power with radius (e.g., in a homogenous medium). Considering a case of a 4 mm diameter electrode, the Joule heating at a location 1 mm from the electrode surface will be over 5× (i.e., five times) smaller than at the surface itself. Equivalently, it follows that if the electrode is spaced by 1 mm from a tissue surface, approximately 5× (i.e., five times) more PFA pulses could be delivered within the same period of time before causing equivalent energy delivery at the tissue surface. In practice, determination of the temperature field may require consideration of thermal diffusion effects of a Joule heating field. However, the above example illustrates the principle as it applies to avoiding thermal damage to tissue during PFA delivery.

In the above example, some consideration may also be applied to thermal effects on blood. In cardiac applications, one thermal concern is the risk of downstream embolism from thermal coagulation of blood. However, this risk is typically mitigated by the presence of blood flow. This risk is also additionally mitigated by the relatively higher temperature for thermal coagulation of blood (e.g., as compared with typical thermal ablation temperatures of cardiac tissue). Blood flow has the benefit of cooling the electrode itself (e.g., blood flow may provide convective heat transfer coefficients in the range of 510-4800 W m⁻² K⁻¹). The significant heating of blood is also reduced due to the blood flow itself, which moves heated blood away from the electrode and replaces it with body temperature blood. For example, it may be estimated that a mean atrial flow speed of 0.2 m/s will, in free stream flow, cause heated blood to have moved 1 mm within just 50 milliseconds, essentially fully replacing blood near the electrode between each heartbeat. Boundary layer effects may cause less motion than illustrated above, but the principle is illustrated. In essence, thermal effects arising from PFA have little to no opportunity to accumulate within the blood. In contrast, when contact is maintained with tissue, heat from each PFA pulse is additive within the tissue. Therefore, thermal coagulation of blood would only be a concern where the energy delivered is sufficient to cause immediate thermal coagulation before the blood has become replaced (e.g., within one heartbeat). Lastly, thermal coagulation of blood occurs at a higher temperature than that required to cause thermal damage to tissue. Blood typically coagulates at a temperature range of 65-85° C., whereas cardiac tissue cell death can occur with an exposure of just 2-3 seconds at 57° C. Therefore, it is suggested that additional PFA pulses when in poor tissue contact with intervening blood would be possible even without the benefit of cooling by blood convection. All of these factors together result in a capacity to increase the number of PFA pulses that may be delivered per unit time without exceeding blood damage limits when lesser degrees of PFA electrode-to-tissue contact are present (e.g., when the PFA electrode is separated from the tissue within flowing blood).

It is noted that thermal ablation techniques (i.e., ablation techniques in which tissue cell death is accomplished predominantly via thermal means) such as RF ablation (as opposed to pulsed field ablation), due to their relatively longer energy application times can ultimately lead to thermal coagulation of blood when low electrode-to-tissue contact is present. Attempts to compensate for poor or insufficient electrode-to-tissue contact in thermal ablation applications (e.g., RF ablation) by increasing power are limited by the need to prevent this thermal coagulation of blood. When an electrode is elevated above the tissue, the extra margin for RF energy delivery due to higher flow away from tissue may not be enough to compensate for the much higher power required to cause thermal injury to the non-contacting target tissue, since Joule heating diminishes very rapidly with distance from an electrode (e.g., decreasing with the fourth power for a spherical electrode in homogenous media). Consequently, in thermal ablation procedures, the required delivered power increases massively with increased distance from the tissue, all in order to deposit the same energy within the tissue. In thermal ablation procedures, Joule heating at the surface of the electrode is therefore also equally severely increased relative to when the electrode is in contact with tissue, and in that way blood thermal coagulum limits are soon met rendering it unsafe to attempt to compensate for loss of contact. In PFA, on the other hand, lesion creation is associated with a particular electric field strength or voltage gradient (i.e., V/cm). The threshold voltage gradient for lesion formation is a decreasing monotonic function of the number of pulses delivered. For example, the voltage gradient associated with a spherical electrode may be provided by an inverse square relationship, rather than a fourth power relationship as for Joule heating. Therefore, depending on the specific relationship between PFA pulse count and lethal voltage gradient threshold (e.g., a voltage gradient threshold sufficient to cause tissue death via irreversible electroporation), relatively small changes in the required voltage gradient threshold, caused by increasing PFA pulse count, can move the lesion creation distance from the electrode substantially away via the inverse square relationship without necessarily resulting in a severe increase in near-electrode Joule heating. This ability may depend on the specific sensitivity of the lethal threshold to additional pulsing. However, what should be clear is that this relationship is independent of the physics of Joule heating, in contrast to RF ablation with additional applied power.

To elaborate, during PFA, the energy is delivered at a rate intended to prevent thermal damage to the target tissue at the surface or near the surface, and this energy is a far lower power output compared to RF thermal ablation, meaning there is ample margin for additional power deposition before thermal coagulation is an issue. Consequently, when a PFA electrode instead is spaced from tissue, there will be a large margin to increase the pulse rate before thermal coagulation is a concern, provided other factors such as microbubble formation do not become limiting instead. PFA is driven by voltage gradient, rather than Joule heating, and so the effectiveness of additional pulses when not in contact are driven by the specific relationship between pulse count and the threshold voltage gradient for cell death; Joule heating of both blood and tissue are only secondary consequences.

With this context in mind, in some embodiments, the high voltage pulse train is a first high voltage pulse train of a plurality of high voltage pulse trains, and the data processing device system (e.g., 110, 310) is configured at least by the program at least to cause activation, via the input-output device system (e.g., 120, 320), of the particular (e.g., selected) pulsed field ablation transducer set to deliver each high voltage pulse train of the plurality of high voltage pulse trains during a respective cardiac cycle of a plurality of cardiac cycles. In some embodiments, in response to the first state (e.g., the first state referred to in block 664a in FIG. 6G) in which the detected degree of tissue contact is the first degree, the data processing device system (e.g., 110, 310) is configured at least by the program at least to cause the activation (e.g., the activation per block 664), via the input-output device system (e.g., 120, 320), of the particular pulsed field ablation transducer set to deliver each high voltage pulse train of the plurality of high voltage pulse trains during the respective cardiac cycle of a plurality of cardiac cycles in accordance with the first particular parameter set. In some embodiments, in response to the second state (e.g., the second state referred to in block 664b in FIG. 6G) in which the detected degree of tissue contact is the second degree, the data processing device system (e.g., 110, 310) is configured at least by the program at least to cause the activation (e.g., the activation per block 664), via the input-output device system (e.g., 120, 320), of the particular pulsed field ablation transducer set to deliver each high voltage pulse train of the plurality of high voltage pulse trains during the respective cardiac cycle of a plurality of cardiac cycles in accordance with the second particular parameter set.

In some embodiments, the data processing device system (e.g., 110, 310) is configured at least by the program at least to cause the high voltage pulse train to deliver, in the first state, a first average power in accordance with the first particular parameter set, and cause the high voltage pulse train to deliver, in the second state, a second average power in accordance with the second particular parameter set, wherein the second average power is within 10% of the first average power.

According to various embodiments, the high voltage pulses of the high voltage pulse train delivered in accordance with the first particular parameter set collectively deliver first energy, and the high voltage pulses of the high voltage pulse train delivered in accordance with the second particular parameter set collectively deliver second energy. According to various embodiments, the first energy is greater than the second energy.

These different pulse train delivery characteristics may be one or more of a variety of characteristics in various embodiments. In some embodiments, each of the first particular parameter set and the second particular parameter set defines a respective pulse frequency of the pulses in the high voltage pulse train. In some embodiments, the respective pulse frequency of the pulses in the high voltage pulse train defined in accordance with the first particular parameter set is greater than the respective pulse frequency of the pulses in the high voltage pulse train defined in accordance with the second particular parameter set. For example, a greater PFA pulse frequency may be applied in instances of relatively lesser tissue contact. Similarly, in some embodiments, each of the first particular parameter set and the second particular parameter set defines a respective number of pulses in the high voltage pulse train. In some embodiments, the respective number of pulses in the high voltage pulse train defined in accordance with the first particular parameter set is greater than the respective number of pulses in the high voltage pulse train defined in accordance with the second particular parameter set.

In some embodiments, each of the first particular parameter set and the second particular parameter set defines a respective pulse duration of each of at least one high voltage pulse in the high voltage pulse train. In some embodiments, the respective pulse duration of each of the at least one high voltage pulse in the high voltage pulse train defined in accordance with the first particular parameter set is greater than the respective pulse duration of each of the at least one high voltage pulse in the high voltage pulse train defined in accordance with the second particular parameter set. In some embodiments, the respective pulse duration of each high voltage pulse in the high voltage pulse train defined in accordance with the first particular parameter set is greater than the respective pulse duration of each high voltage pulse in the high voltage pulse train defined in accordance with the second particular parameter set. For example, in some embodiments, a greater PFA pulse duration or more PFA pulse energy may be applied in instances of relatively lesser tissue contact.

In some embodiments, each of the first particular parameter set and the second particular parameter set defines a respective pulse amplitude (also known as a pulse voltage in some embodiments) of each of at least one high voltage pulse in the high voltage pulse train. In some embodiments, the respective pulse amplitude (e.g., pulse voltage) of each of the at least one high voltage pulse in the high voltage pulse train defined in accordance with the first particular parameter set is greater than the respective pulse amplitude of each of the at least one high voltage pulse in the high voltage pulse train defined in accordance with the second particular parameter set. In some embodiments, the respective pulse amplitude of each high voltage pulse in the high voltage pulse train defined in accordance with the first particular parameter set is greater than the respective pulse amplitude of each high voltage pulse in the high voltage pulse train defined in accordance with the second particular parameter set. For example, in some embodiments, a greater PFA pulse amplitude or more PFA pulse energy may be applied in instances of relatively lesser tissue contact.

It is noted that consideration of other factors may have a bearing on the various first parameter sets and second parameter sets described above according to various embodiments. For example, increasing PFA pulse duration or increasing pulse amplitude/voltage in response to detected relatively low degrees of tissue contact may be limited by muscle contraction considerations and microbubble considerations, according to various embodiments. In some embodiments, different respective factors may come into play for different detected degrees of tissue contact. For example, in some embodiments, when a relatively low detected degree of tissue contact is detected, increased numbers of PFA pulses (or increased pulse frequency) may be employed, the increased numbers of PFA pulses (or increased pulse frequency) limited microbubble limits. When relatively higher degrees of tissue contact, are detected, factors such as tissue temperature rise limits may limit the number of PFA pulses or pulse frequency that may be achieved (e.g., as compared with the response to a relatively low detected degree of tissue contact is detected).

In some embodiments, each high voltage pulse in the high voltage pulse train is configured to deliver a respective amount of pulse energy. In some embodiments, the pulse energy deliverable by each of at least one high voltage pulse in the high voltage pulse train in accordance with the first particular parameter set is greater than the pulse energy deliverable by each of at least one high voltage pulse in the high voltage pulse train in accordance with the second particular parameter set. In some embodiments, the pulse energy deliverable by each high voltage pulse in the high voltage pulse train in accordance with the first particular parameter set is greater than the pulse energy deliverable by each high voltage pulse in the high voltage pulse train in accordance with the second particular parameter set. For example, in some embodiments, a greater PFA pulse energy may be applied in instances of relatively lesser tissue contact.

In some embodiments, the degree of tissue contact exhibited by a portion of a catheter includes a degree of tissue contact exhibited by a portion of the catheter device that is provided by one or more transducers of the catheter device, the one or more transducers configured to be positioned within a body of a patient. In some embodiments, the data processing device system (e.g., 110, 310) is configured to cause the detection, via the input-output device system (e.g., 120, 320), of the degree of tissue contact exhibited by the portion of the catheter device at least in part from a signal set provided by one or more transducers, the one or more transducers configured to be positioned within a body of a patient. In some embodiments, the one or more transducers are provided by the catheter device.

In some embodiments, the degree of tissue contact exhibited by a portion of a catheter includes a degree of tissue contact exhibited by a portion of the catheter device that is provided by one or more pulsed field ablation transducers of the catheter device. In some embodiments, the particular pulsed field ablation transducer set includes the one or more pulsed field ablation transducers of the catheter device activated in accordance with block 664.

In some embodiments, each pulsed field ablation transducer of the catheter device comprises a respective electrode (e.g., 315), each respective electrode including a respective energy delivery surface (e.g., 319) configured to deliver pulsed field ablation energy. In some embodiments, the data processing device system (e.g., 110, 310) is configured to cause the detection, via the input-output device system (e.g., 120, 320), of the degree of tissue contact exhibited by the portion of the catheter device at least by causing detection, via the input-output device system (e.g., 120, 320), of a degree of tissue contact exhibited by at least a part of the respective energy delivery surface (e.g., 319) of each of at least some of the pulsed field ablation transducers of the catheter device.

While some of the embodiments disclosed above are described with examples of cardiac mapping, ablation, or both, the same or similar embodiments may be used for mapping, ablating, or both, other bodily organs, for example with respect to the intestines, the bladder, or any bodily organ to which the devices of the present invention may be introduced.

Subsets or combinations of various embodiments described above can provide further embodiments.

These and other changes can be made to the invention in light of the above-detailed description. In general, in the following claims, the terms used should not be construed to limit the invention to the specific embodiments disclosed in the specification and the claims, but should be construed to include other transducer-based device systems including all medical treatment device systems and all medical diagnostic device systems in accordance with the claims. Accordingly, the invention is not limited by the disclosure.

What is claimed is:

1. A pulsed field ablation system comprising:
an input-output device system;
a memory device system storing a program; and
a data processing device system communicatively connected to the input-output device system and the memory device system, the data processing device system configured at least by the program at least to:
cause, in association with a first state in which a first plurality of consecutive cardiac cycles of a patient exhibit a non-irregular heart rate, a first pulsed field ablation transducer located on a catheter device to deliver pulsed field ablation energy during each of some, but not all, of the first plurality of consecutive cardiac cycles, the some, but not all, of the first plurality of consecutive cardiac cycles excluding at least one cardiac cycle of the first plurality of consecutive cardiac cycles during which no pulsed field ablation energy is delivered by the first pulsed field ablation transducer, the excluded at least one cardiac cycle of the first plurality of consecutive cardiac cycles occurring between at least two cardiac cycles of the some, but not all, of the first plurality of consecutive cardiac cycles.

2. The pulsed field ablation system of claim 1, wherein the non-irregular heart rate is a constant heart rate.

3. A pulsed field ablation system comprising:
an input-output device system;
a memory device system storing a program; and
a data processing device system communicatively connected to the input-output device system and the memory device system, the data processing device system configured at least by the program at least to:
identify a particular pulsed field ablation transducer set of a catheter device, the particular pulsed field ablation transducer set identified from a plurality of pulsed field ablation transducers of the catheter device, and the particular pulsed field ablation transducer set identified to be activated to apply a high voltage pulse train between the pulsed field ablation transducers of the particular pulsed field ablation transducer set, the high voltage pulse train sufficient to cause pulsed field ablation of tissue;
in association with a first state in which the identified particular pulsed field ablation transducer set is a first set of pulsed field ablation transducers of the catheter device, determine a first particular parameter set of the high voltage pulse train and cause activation, via the input-output device system, of the identified particular pulsed field ablation transducer set to deliver the high voltage pulse train in accordance with the determined first particular parameter set; and
in association with a second state in which the identified particular pulsed field ablation transducer set is a second set of pulsed field ablation transducers of the catheter device different than the first set of pulsed field ablation transducers, determine a second particular parameter set of the high voltage pulse train different than the first particular parameter set and cause activation, via the input-output device system, of the identified particular pulsed field ablation transducer set to deliver the high voltage pulse train in accordance with the determined second particular parameter set.

4. The pulsed field ablation system of claim 3,
wherein, in the first state in which the identified particular pulsed field ablation transducer set is the first set of pulsed field ablation transducers of the catheter device, the first set of pulsed field ablation transducers has a first number of pulsed field ablation transducers, and
wherein, in the second state in which the identified particular pulsed field ablation transducer set is the second set of pulsed field ablation transducers of the catheter device, the second set of pulsed field ablation transducers has a second number of pulsed field ablation transducers greater than the first number of pulsed field ablation transducers.

5. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy,
wherein, in the first state in which the identified particular pulsed field ablation transducer set is the first set of pulsed field ablation transducers of the catheter device, the energy delivery surfaces of the first set of pulsed field ablation transducers have a first collective area, and
wherein, in the second state in which the identified particular pulsed field ablation transducer set is the second set of pulsed field ablation transducers of the catheter device, the energy delivery surfaces of the second set of pulsed field ablation transducers have a second collective area greater than the first collective area.

6. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy,
wherein, in the first state in which the identified particular pulsed field ablation transducer set is the first set of pulsed field ablation transducers of the catheter device, the energy delivery surfaces of the first set of pulsed field ablation transducers have a first set of one or more geometric shapes, and
wherein, in the second state in which the identified particular pulsed field ablation transducer set is the second set of pulsed field ablation transducers of the catheter device, the energy delivery surfaces of the second set of pulsed field ablation transducers have a second set of one or more geometric shapes different than the first set of one or more geometric shapes.

7. The pulsed field ablation system of claim 3, wherein the particular pulsed field ablation transducer set is identified based at least on a selection of at least two pulsed field ablation transducers of the catheter device, each pulsed field ablation transducer of the at least two pulsed field ablation transducers configured to selectively deliver energy sufficient for pulsed field ablation of tissue.

8. The pulsed field ablation system of claim 7, wherein the selection of the at least two pulsed field ablation transducers of the catheter device is a user selection of the at least two pulsed field ablation transducers.

9. The pulsed field ablation system of claim 3, wherein the data processing device system is configured at least by the program at least to perform an analysis of a total number of at least the pulsed field ablation transducers in the particular pulsed field ablation transducer set.

10. The pulsed field ablation system of claim 9,
wherein, in the first state, the analysis of the total number of the at least the pulsed field ablation transducers in the particular pulsed field ablation transducer set is an analysis of a total number of pulsed field ablation transducers in the first set of pulsed field ablation transducers,
wherein, in the second state, the analysis of the total number of the at least the pulsed field ablation transducers in the particular pulsed field ablation transducer set is an analysis of a total number of pulsed field ablation transducers in the second set of pulsed field ablation transducers,
wherein, in the first state, the first particular parameter set of the high voltage pulse train is determined based at least on the analysis of the total number of pulsed field ablation transducers in the first set of pulsed field ablation transducers, and
wherein, in the second state, the second particular parameter set of the high voltage pulse train is determined based at least on the analysis of the total number of pulsed field ablation transducers in the second set of pulsed field ablation transducers.

11. The pulsed field ablation system of claim 3, wherein the data processing device system is configured at least by the program at least to perform an analysis of a transducer type of each pulsed field ablation transducer of at least the pulsed field ablation transducers of the particular pulsed field ablation transducer set.

12. The pulsed field ablation system of claim 11,
wherein, in the first state, the analysis of a transducer type of each pulsed field ablation transducer in the at least the pulsed field ablation transducers in the particular pulsed field ablation transducer set is an analysis of a transducer type of each pulsed field ablation transducer in the first set of pulsed field ablation transducers,
wherein, in the second state, the analysis of a transducer type of each pulsed field ablation transducer in the at least the pulsed field ablation transducers in the particular pulsed field ablation transducer set is an analysis of a transducer type of each pulsed field ablation transducer in the second set of pulsed field ablation transducers,
wherein, in the first state, the first particular parameter set of the high voltage pulse train is determined based at least on the analysis of a transducer type of each pulsed field ablation transducer in the first set of pulsed field ablation transducers, and
wherein, in the second state, the second particular parameter set of the high voltage pulse train is determined based at least on the analysis of a transducer type of each pulsed field ablation transducer in the second set of pulsed field ablation transducers.

13. The pulsed field ablation system of claim 3, wherein the data processing device system is configured at least by the program at least to perform an analysis of size, shape, or size and shape of each pulsed field ablation transducer of at least the pulsed field ablation transducers of the particular pulsed field ablation transducer set.

14. The pulsed field ablation system of claim 13,
wherein, in the first state, the analysis of size, shape, or size and shape of each pulsed field ablation transducer in the at least the pulsed field ablation transducers in the particular pulsed field ablation transducer set is an analysis of size, shape, or size and shape of each pulsed field ablation transducer in the first set of pulsed field ablation transducers,
wherein, in the second state, the analysis of size, shape, or size and shape of each pulsed field ablation transducer in the at least the pulsed field ablation transducers in the particular pulsed field ablation transducer set is an analysis of size, shape, or size and shape of each pulsed field ablation transducer in the second set of pulsed field ablation transducers,
wherein, in the first state, the first particular parameter set of the high voltage pulse train is determined based at least on the analysis of size, shape, or size and shape of each pulsed field ablation transducer in the first set of pulsed field ablation transducers, and
wherein, in the second state, the second particular parameter set of the high voltage pulse train is determined based at least on the analysis of size, shape, or size and shape of each pulsed field ablation transducer in the second set of pulsed field ablation transducers.

15. The pulsed field ablation system of claim 3, wherein the particular pulsed field ablation transducer set is identified based at least on an analysis of degree of tissue contact exhibited by at least the pulsed field ablation transducers of the particular pulsed field ablation transducer set.

16. The pulsed field ablation system of claim 3, wherein the particular pulsed field ablation transducer set is identified based at least on an analysis of data provided by each pulsed field ablation transducer of at least the pulsed field ablation transducers of the particular pulsed field ablation transducer set.

17. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy, and
wherein (a) in the first state in which the identified particular pulsed field ablation transducer set is the first set of pulsed field ablation transducers of the catheter device, the energy delivery surfaces of the first set of pulsed field ablation transducers have a same area, or (b) in the second state in which the identified particular pulsed field ablation transducer set is the second set of transducers of the catheter device, the energy delivery surfaces of the second set of transducers have a same area.

18. The pulsed field ablation system of claim 17,
wherein each pulsed field ablation transducer of the identified particular pulsed field ablation transducer set comprises a respective electrode, each respective electrode including a respective energy delivery surface configured to deliver pulsed field ablation energy, and
wherein (c) in the first state in which the identified particular pulsed field ablation transducer set is the first set of pulsed field ablation transducers of the catheter device, the energy delivery surfaces of the first set of pulsed field ablation transducers have a same geometric shape, or (d) in the second state in which the identified particular pulsed field ablation transducer set is the second set of pulsed field ablation transducers of the catheter device, the energy delivery surfaces of the second set of pulsed field ablation transducers have a same geometric shape.

19. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy,
wherein, in the first state in which the identified particular pulsed field ablation transducer set is the first set of pulsed field ablation transducers of the catheter device, each energy delivery surface of at least one energy delivery surface of the first set of pulsed field ablation transducers has a first area, and
wherein, in the second state in which the identified particular pulsed field ablation transducer set is the second set of pulsed field ablation transducers of the catheter device, each energy delivery surface of at least one energy delivery surface of the second set of pulsed field ablation transducers has a second area different than the first area.

20. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy,
wherein, in the first state in which the identified particular pulsed field ablation transducer set is the first set of pulsed field ablation transducers of the catheter device, the energy delivery surface of each of at least one pulsed field ablation transducer of the first set of pulsed field ablation transducers has a first area, and
wherein, in the second state in which the identified particular pulsed field ablation transducer set is the second set of pulsed field ablation transducers of the catheter device, the energy delivery surfaces of each of at least one pulsed filed ablation transducer of the second set of pulsed field ablation transducers has a second area the same as the first area.

21. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy, and
wherein, each energy delivery surface of the first set of pulsed field ablation transducers in the first state has a different area than each energy delivery surface of the second set of pulsed field ablation transducers in the second state.

22. The pulsed field ablation system of claim 3,
wherein each pulsed field ablation transducer of the identified particular pulsed field ablation transducer set comprises a respective electrode, each respective electrode including a respective energy delivery surface configured to deliver pulsed field ablation energy,
wherein, in the first state in which the identified particular pulsed field ablation transducer set is the first set of pulsed field ablation transducers of the catheter device, each energy delivery surface of at least one energy delivery surface of the first set of pulsed field ablation transducers has a first geometric shape, and
wherein, in the second state in which the identified particular pulsed field ablation transducer set is the second set of pulsed field ablation transducers of the catheter device, each energy delivery surface of at least one energy delivery surface of the second set of pulsed field ablation transducers has a second geometric shape different than the first geometric shape.

23. The pulsed field ablation system of claim 19, wherein the respective energy delivery surfaces of the first set of pulsed field ablation transducers in the first state have a same area.

24. The pulsed field ablation system of claim 23, wherein the respective energy delivery surfaces of the second set of transducers in the second state have a same area.

25. The pulsed field ablation system of claim 3, wherein each high voltage pulse in the high voltage pulse train is configured to deliver a respective amount of pulse energy, and wherein the pulse energy deliverable by each of at least one high voltage pulse in the high voltage pulse train in accordance with the second particular parameter set is less than the pulse energy deliverable by each of at least one high voltage pulse in the high voltage pulse train in accordance with the first particular parameter set.

26. The pulsed field ablation system of claim 3, wherein each high voltage pulse in the high voltage pulse train comprises a respective rise time, and wherein the respective rise time of each high voltage pulse of the high voltage pulse train in accordance with the second particular parameter set is longer than the respective rise time of each high voltage pulse of the high voltage pulse train in accordance with the first particular parameter set.

27. The pulsed field ablation system of claim 3, wherein each of the first particular parameter set and the second particular parameter set defines a respective pulse duration of each of at least one high voltage pulse in the high voltage pulse train, and wherein the respective pulse duration of each of the at least one high voltage pulse in the high voltage pulse train defined in accordance with the second particular parameter set is less than the respective pulse duration of each of the at least one high voltage pulse in the high voltage pulse train defined in accordance with the first particular parameter set.

28. The pulsed field ablation system of claim 3,wherein each of the first particular parameter set and the second particular parameter set defines a respective pulse frequency of the pulses in the high voltage pulse train, and wherein the respective pulse frequency of the pulses in the high voltage pulse train defined in accordance with the second particular parameter set is lower than the respective pulse frequency of the pulses in the high voltage pulse train defined in accordance with the first particular parameter set.

29. The pulsed field ablation system of claim 3, wherein each of the first particular parameter set and the second particular parameter set defines a respective number of pulses in the high voltage pulse train, and wherein the respective number of pulses in the high voltage pulse train defined in accordance with the second particular parameter set is less than the respective number of pulses in the high voltage pulse train defined in accordance with the first particular parameter set.

30. The pulsed field ablation system of claim 3, wherein the data processing device system is configured at least by the program at least to cause the high voltage pulse train to deliver, in the first state, a first average power in accordance with the first particular parameter set, and cause the high voltage pulse train to deliver, in the second state, a second average power in accordance with the second particular parameter set, wherein the second average power is within 10% of the first average power.

31. The pulsed field ablation system of claim 3, wherein the high voltage pulse train is a first high voltage pulse train of a plurality of high voltage pulse trains, wherein the data processing device system is configured at least by the program at least to cause activation, via the input-output device system, of the particular pulsed field ablation transducer set to deliver each high voltage pulse train of the plurality of high voltage pulse trains during a respective cardiac cycle of a plurality of cardiac cycles.

32. The pulsed field ablation system of claim 3,
wherein the determination of the first particular parameter set includes a delivery of a first preliminary or test signal set between the pulsed field ablation transducers in the first set of pulsed field ablation transducers, and
wherein the determination of the second particular parameter set includes a delivery of a second preliminary or test signal set between the pulsed field ablation transducers in the first set of pulsed field ablation transducers.
